(19) United States
(12) Patent Application Publication
(10) Pub. No.: US 2024/0200155 A1
(43) Pub. Date: Jun. 20, 2024

(54) METHODS OF USING TOMATO BROWN RUGOSE FRUIT VIRUS AS AN INDICATOR OF FECAL STRENGTH AND CONTAMINATION, AND AS A CONTROL FOR VIRAL RNA EXTRACTION FROM STOOL

(71) Applicant: The Board of Trustees of the Leland Stanford Junior University, Stanford, CA (US)

(72) Inventors: Alexandria B. Boehm, Redwood City, CA (US); Aravind Natarajan, Redwood City, CA (US); Brayon J. Fremin, Redwood City, CA (US); Marlene Wolfe, Redwood City, CA (US)

(21) Appl. No.: 18/540,291

(22) Filed: Dec. 14, 2023

Related U.S. Application Data
(60) Provisional application No. 63/387,657, filed on Dec. 15, 2022.

(51) Int. Cl. C12Q 1/70

(52) U.S. Cl. CPC C12Q 1/701; C12Q 2600/16

(57) ABSTRACT

Compositions and methods are provided for detecting the tomato brown rugose fruit virus (ToBRFV), which is highly abundant in human stool and wastewater. In particular, methods of using ToBRFV in microbial source tracking as a marker of human fecal contamination are disclosed. The methods utilize primers and probes capable of amplifying and/or detecting target ToBRFV sequences in samples suspected of having human fecal contamination. Methods of using ToBRFV in wastewater surveillance of pathogens as a marker to quantitate feces are also provided.

### CROSS-REFERENCE TO RELATED APPLICATION

This application claims benefit under 35 U.S.C. § 119(e) of provisional application 63/387,657, filed Dec. 15, 2022, which application is hereby incorporated by reference in its entirety.

### INCORPORATION BY REFERENCE OF A SEQUENCE LISTING

A Sequence Listing is provided herewith as a Sequence Listing XML file, “STAN-2060”, created on Dec. 8, 2023, and having a size of 25,158 bytes. The contents of the Sequence Listing XML file are incorporated by reference herein in their entirety.

### BACKGROUND OF THE INVENTION

Across the world, water quality is assessed for human fecal contamination using microbial indicators, including total coliforms, Escherichia coli, and enterococci (Whitman et al. 2003). For instance, in the United States of America (U.S.A.), drinking water and recreational water quality standards require the abundance of these organisms be less than prescribed levels (United States Environmental Protection Agency Office of Water (US EPA OW) (2013) Recreational Water Quality Criteria and Methods, US EPA OW (2015) Drinking Water Regulations). Using these organisms to assess water quality is advantageous because they are generally not pathogenic, and are highly abundant in human feces leading to their utility in detecting even trace contamination of waterbodies. Additionally, their presence may indicate the potential contamination of waterbodies by other sparser human pathogens that may be harder to detect. However, there are some limitations to their utility. These microbial indicators of human fecal contamination are also found in non-human feces. Additionally, they can be present and even grow in the environment, including in decaying plant material (Imamura et al. (2011) FEMS Microbiol Ecol 77:40-49; Whitman et al. (2003) Appl. Environ. Microbiol. 69:4714-4719), and in soils and sands (Yamahara K M, Walters S P, Boehm A B. 2009. Growth of enterococci in unaltered, unseeded beach sands subjected to tidal wetting. Appl Environ Microbiol 75:1517-1524; Byappanahalli et al. (2006) Environ. Microbiol. 8:504-513). Therefore, there is a growing need to identify new microbial indicator targets that can be used to assess the presence of human fecal contamination exclusively.

### SUMMARY OF THE INVENTION

Compositions and methods are provided for detecting the tomato brown rugose fruit virus (ToBRFV), which is highly abundant in human stool and wastewater. In particular, methods of using ToBRFV in microbial source tracking as a marker of human fecal contamination are disclosed. Methods of using ToBRFV in wastewater surveillance of pathogens as a marker to quantitate feces are also provided. The methods utilize primers and probes capable of amplifying and/or detecting target ToBRFV sequences in samples suspected of having human fecal contamination. In certain embodiments, the ToBRFV sequences are detected using droplet digital PCR (ddPCR) or quantitative polymerase chain reaction (qPCR), which also allow quantification of the ToBRFV. Other nucleic-acid based detection techniques such as, but not limited to, reverse transcriptase polymerase chain reaction (RT-PCR), nucleic acid sequence-based amplification (NASBA), transcription-mediated amplification (TMA), and a fluorogenic 5′ nuclease assay can also be used, among others.

In one aspect, a method for selectively detecting tomato brown rugose fruit virus (ToBRFV) in a sample suspected of having human fecal contamination, the method comprising: isolating nucleic acids from the sample suspected of having human fecal contamination, wherein if ToBRFV RNA is present, said nucleic acids comprise a target sequence; amplifying the nucleic acids using a set of primers capable of selectively amplifying at least a portion of the ToBRFV RNA, wherein the ToBRFV RNA comprise the target sequence; and detecting the presence of the amplified nucleic acids using a detectably labeled oligonucleotide probe sufficiently complementary to and capable of hybridizing with the ToBRFV RNA or amplicon thereof, if present, as an indication of the presence or absence of the ToBRFV in the sample, wherein said primers and said probe are capable of selectively hybridizing to the target sequence from the ToBRFV, wherein said detecting the presence of the ToBRFV in the sample indicates that human feces is present in the sample.

In certain embodiments, the method for selectively detecting tomato brown rugose fruit virus (ToBRFV) in a sample suspected of having human fecal contamination comprises: isolating nucleic acids from the sample suspected of having human fecal contamination, wherein if ToBRFV RNA is present, the nucleic acids comprise a target sequence; amplifying the nucleic acids using a set of primers capable of selectively amplifying at least a portion of a movement protein (Mo) encoding gene or at least a portion of an RNA dependent RNA polymerase (RdRP) encoding gene, or a combination thereof; and detecting the presence of the amplified nucleic acids using a detectably labeled oligonucleotide probe sufficiently complementary to and capable of hybridizing with the ToBRFV RNA or amplicon thereof, if present, as an indication of the presence or absence of the ToBRFV in the sample, wherein said primers and said probe are capable of selectively hybridizing to the target sequence from the ToBRFV, wherein said detecting the presence of the ToBRFV in the sample indicates that human feces is present in the sample.

In certain embodiments, the set of primers comprises: (a) a forward primer comprising or consisting of the sequence of SEQ ID NO:1 and a reverse primer comprising or consisting of the sequence of SEQ ID NO:2; (b) a forward primer comprising or consisting of the sequence of SEQ ID NO:4 and a reverse primer comprising or consisting of the sequence of SEQ ID NO:5; (c) a forward primer and a reverse primer each comprising at least 10 contiguous nucleotides from the corresponding nucleotide sequences of the forward primer and reverse primer of a primer set selected from the group consisting of (a) and (b); (d) a forward primer and a reverse primer comprising at least one nucleotide sequence that differs from the corresponding nucleotide sequence of the forward primer or reverse primer of a primer set selected from the group consisting of (a)-(c) in that the primer has up to three nucleotide changes compared to the corresponding sequence, wherein the primer is capable of hybridizing to and amplifying the ToBRFV nucleic acids; (e) a forward primer and a reverse primer comprising or consisting of nucleotide sequences that are complements of the corresponding nucleotide sequences of the forward primer and reverse primer of a primer set selected from the group consisting of (a)-(d); or (f) a combination of a primer set selected from the group consisting of (a)-(e).

In certain embodiments, the set of primers used for detecting ToBRFV in the sample comprises a primer comprising or consisting of the sequence of SEQ ID NO:1, a primer comprising or consisting of the sequence of SEQ ID NO:2, a primer comprising or consisting of the sequence of SEQ ID NO:4, and a primer comprising or consisting of the sequence of SEQ ID NO:5.

In certain embodiments, at least one oligonucleotide probe is selected from the group consisting of: (a) a probe comprising or consisting of the sequence of SEQ ID NO:3, (b) a probe comprising or consisting of a sequence selected from the group consisting of SEQ ID NO:6 and SEQ ID NO:7, (c) a probe that differs from the corresponding nucleotide sequence of a probe selected from the group consisting of (a) and (b) in that the probe has up to three nucleotide changes compared to the corresponding sequence, wherein the probe is capable of hybridizing to and detecting the ToBRFV RNA or amplicon thereof, and (d) a combination of probes selected from the group consisting of (a)-(c).

In certain embodiments, the at least one oligonucleotide probe comprises a probe comprising or consisting of the sequence of SEQ ID NO:3 and a probe comprising or consisting of the sequence selected from the group consisting of SEQ ID NO:6 and SEQ ID NO:7.

In certain embodiments, at least one probe is detectably labeled with a fluorophore. In some embodiments, each probe is detectably labeled with a different fluorophore. In some embodiments, each probe is detectably labeled with a 5′-fluorophore and a 3′-quencher.

In certain embodiments, the probe is not more than about 40 nucleotides in length.

In certain embodiments, the primers are not more than about 40 nucleotides in length.

In certain embodiments, the sample is a stool sample, environmental sample, or fomite sample. Samples may include water samples such as, but not limited to, samples of wastewater, stormwater, ocean water, lake water, river water, creek water, drinking water, recreational water, ground water, source water, stored water, seepage water, surface water, or water from a water distribution system or sewage and waste water treatment system; samples of air potentially containing aerosolized stool matter; earth samples such as, but not limited to, samples of soil, sand, mud, sediment, or rock; sludge samples; or samples from surfaces such as a food preparation surfaces, including, but not limited to, counters, cutting boards, tableware, utensils, measuring cups/spoons, spatulas, dishware, glassware, cutlery, pots and pans, cooking equipment, or tables; or samples from fomite surfaces such as, but not limited to, clothes, bedding, utensils, cups, furniture, vehicles, shovels, bowls/buckets, brushes, tack, clippers, pencils, bathroom faucet handles, toilet flush levers, door knobs, light switches, handrails, elevator buttons, television remote controls, pens, touch screens, common-use phones, keyboards and computer mice, coffeepot handles, countertops, drinking fountains, medical equipment such as, but not limited to, stethoscopes, intravenous (IV) drip tubes, catheters, and life support equipment; and any other items that are frequently touched by different people and infrequently cleaned.

In certain embodiments, amplifying the nucleic acids comprises performing polymerase chain reaction (PCR) or isothermal amplification. In some embodiments, the PCR is reverse transcriptase polymerase chain reaction (RT-PCR), droplet digital polymerase chain reaction (ddPCR), or quantitative PCR (qPCR). In some embodiments, the qPCR uses a fluorogenic 5′ nuclease assay. In some embodiments, the isothermal amplification is nucleic acid sequence-based amplification (NASBA), transcription-mediated amplification (TMA), recombinase polymerase amplification (RPA), loop-mediated isothermal amplification (LAMP), strand displacement amplification (SDA), helicase-dependent amplification (HDA), nicking enzyme amplification reaction (NEAR), ligase chain reaction (LGR), or Q-beta amplification.

In certain embodiments, the method further comprises quantifying the amount of the ToBRFV RNA or amplicon thereof, wherein the amount of the ToBRFV RNA or amplicon thereof is indicative of the amount of the ToBRFV in the sample.

In certain embodiments, the method further comprises correlating the amount of the ToBRFV in the sample with an amount of human feces in the sample.

In certain embodiments, the method further comprises detecting a pathogen in the sample, wherein the amount of ToBRFV in the sample is used to estimate the amount of the pathogen per the amount of the human feces in the sample.

In certain embodiments, the method further comprises detecting a pathogen (e.g., a virus, a bacterium, a fungus, or a parasite) in the sample, wherein the amount of ToBRFV in the sample is used to estimate the amount of the pathogen per the amount of the human feces in the sample.

In certain embodiments, the method further comprises performing microbial source tracking (MST), wherein the ToBRFV is used as a marker of human feces.

In certain embodiments, the ToBRFV is used as a marker to distinguish fecal contamination from human and non-human animals.

In another aspect, an isolated oligonucleotide not more than 60 nucleotides in length comprising: (a) a nucleotide sequence comprising at least 10 contiguous nucleotides from any one of SEQ ID NOS:1-6; (b) a nucleotide sequence having 90% sequence identity to a nucleotide sequence of (a); or (c) complements of (a) and (b) is provided.

In certain embodiments, the oligonucleotide further comprises a detectable label.

In another aspect, a composition is provided, the composition comprising: (a) a forward primer comprising or consisting of the sequence of SEQ ID NO:1 and a reverse primer comprising or consisting of the sequence of SEQ ID NO:2; (b) a forward primer comprising or consisting of the sequence of SEQ ID NO:4 and a reverse primer comprising or consisting of the sequence of SEQ ID NO:5; (c) a forward primer and a reverse primer each comprising at least 10 contiguous nucleotides from the corresponding nucleotide sequences of the forward primer and reverse primer of a primer set selected from the group consisting of (a) and (b); (d) a forward primer and a reverse primer comprising at least one nucleotide sequence that differs from the corresponding nucleotide sequence of the forward primer or reverse primer of a primer set selected from the group consisting of (a)-(c) in that the primer has up to three nucleotide changes compared to the corresponding sequence, wherein the primer is capable of hybridizing to and amplifying the ToBRFV nucleic acids; (e) a forward primer and a reverse primer comprising or consisting of nucleotide sequences that are complements of the corresponding nucleotide sequences of the forward primer and reverse primer of a primer set selected from the group consisting of (a)-(d); or (f) a combination of a primer set selected from the group consisting of (a)-(e).

In certain embodiments, the composition further comprises at least one oligonucleotide probe selected from the group consisting of: (a) a probe comprising or consisting of the sequence of SEQ ID NO:3, (b) a probe comprising or consisting of a sequence selected from the group consisting of SEQ ID NO:6 and SEQ ID NO:7, (c) a probe that differs from the corresponding nucleotide sequence of a probe selected from the group consisting of (a) and (b) in that the probe has up to three nucleotide changes compared to the corresponding sequence, wherein the probe is capable of hybridizing to and detecting the ToBRFV RNA or amplicon thereof, and (d) a combination of probes selected from the group consisting of (a)-(c).

In certain embodiments, the composition comprises: (a) a forward primer comprising or consisting of the sequence of SEQ ID NO:1, a reverse primer comprising or consisting of the sequence of SEQ ID NO:2, and a probe comprising or consisting of the sequence of SEQ ID NO:3; and (b) a forward primer comprising or consisting of the sequence of SEQ ID NO:4, a reverse primer comprising or consisting of the sequence of SEQ ID NO:5, and a probe comprising or consisting of a sequence selected from the group consisting of SEQ ID NO:6 and SEQ ID NO:7.

In another aspect, a method of performing wastewater surveillance of a pathogen is provided, the method comprising: isolating nucleic acids from a sample of wastewater comprising human feces, wherein if ToBRFV RNA is present, said nucleic acids comprise a target sequence; amplifying the nucleic acids using a set of primers capable of selectively amplifying at least a portion of a movement protein (Mo) encoding gene or at least a portion of an RNA dependent RNA polymerase (RdRP) encoding gene; and detecting the presence of the amplified nucleic acids using a detectably labeled oligonucleotide probe sufficiently complementary to and capable of hybridizing with the ToBRFV RNA or amplicon thereof, if present, as an indication of the presence or absence of the ToBRFV in the sample of wastewater, wherein said primers and said probe are capable of selectively hybridizing to the target sequence from the ToBRFV, wherein said detecting the presence of the ToBRFV in the sample of wastewater indicates that human feces is present in the sample; quantifying the amount of the ToBRFV RNA or amplicon thereof, wherein the amount of the ToBRFV RNA or amplicon thereof is indicative of the amount of the human feces in the sample of wastewater; quantifying the amount of the pathogen in the sample of wastewater; and dividing the amount of the pathogen by the amount of the human feces, as determined from said quantifying the amount of the ToBRFV RNA or amplicon thereof, to provide a normalized value for the amount of the pathogen per the amount of human feces in the wastewater.

In certain embodiments, the method further comprises correlating the normalized value for the amount of the pathogen per the amount of human feces in the wastewater with incidence of an infectious disease caused by the pathogen.

In certain embodiments, the method is repeated to monitor the amount of the pathogen in the wastewater over time.

In certain embodiments, quantifying the amount of the pathogen comprises quantifying the amount of pathogen DNA, pathogen RNA, or a pathogen biomarker in the sample of wastewater.

In certain embodiments, the pathogen is a virus, a bacterium, a fungus, or a parasite. In some embodiments, the virus is an enterovirus (e.g., poliovirus, coxsackie A virus, coxsackie B virus, or echovirus), a rotavirus, a parvovirus-like virus, an astrovirus, a calicivirus, an adenovirus, a norovirus, or a coronavirus (e.g., severe acute respiratory syndrome coronavirus 2 (SARS-CoV-2)). In some embodiments, the virus is hepatitis A virus, hepatitis B virus, hepatitis E virus, herpesvirus, influenza A, influenza B, respiratory syncytial virus (RSV), monkeypox virus (MPXV), dengue virus, yellow fever virus, zika virus, or varicella-zoster virus. In some embodiments, the bacterium is Escherichia coli, Salmonella, Shigella, Vibrio, Campylobacter, Yersinia, or Clostridium. In some embodiments, the bacterium is an antibiotic-resistant bacterium. In some embodiments, the fungus is Candida auris, Blastomyces dermatitidis, Blastomyces gilchristii, or Cryptococcus neoformans. In some embodiments, the parasite is Entamoeba histolytica, Cryptosporidium parvum, Cyclospora cayetanensis, Giardia duodenalis, or Plasmodium falciparum.

In certain embodiments, the method further comprises amplifying PMMoV nucleic acids using a set of primers capable of selectively amplifying at least a portion of a coat protein (CP) encoding gene of PMMoV; detecting the presence of the amplified PMMoV nucleic acids using a detectably labeled oligonucleotide probe sufficiently complementary to and capable of hybridizing with the PMMoV RNA or amplicon thereof, if present, as an indication of the presence or absence of the PMMoV in the sample of wastewater, wherein said detecting the presence of the PMMoV in combination with the ToBRFV in the sample indicates that human feces is present in the sample of wastewater; quantifying the amount of the ToBRFV RNA or amplicon thereof, wherein the amount of the PMMoV RNA or amplicon thereof in combination with the amount of the ToBRFV RNA or amplicon thereof is indicative of the amount of the human feces in the sample of wastewater; quantifying the amount of the pathogen in the sample of wastewater; and dividing the amount of the pathogen by the amount of the human feces, as determined from said quantifying the amount of the PMMoV RNA or amplicon thereof in combination with the amount of the ToBRFV RNA or amplicon thereof, to provide a normalized value for the amount of the pathogen per the amount of human feces in the wastewater.

In certain embodiments, the set of primers for amplifying the PMMoV nucleic acids comprises: (a) a forward primer comprising or consisting of the sequence of SEQ ID NO:9 and a reverse primer comprising or consisting of the sequence of SEQ ID NO:10; (b) a forward primer comprising at least 10 contiguous nucleotides of the sequence of SEQ ID NO:9 and a reverse primer comprising at least 10 contiguous nucleotides of the sequence of SEQ ID NO:10; (c) a forward primer and a reverse primer comprising at least one nucleotide sequence that differs from the corresponding nucleotide sequence of the forward primer or reverse primer of a primer set selected from the group consisting of (a) and (b) in that the primer has up to three nucleotide changes compared to the corresponding sequence, wherein the primer is capable of hybridizing to and amplifying the PMMoV nucleic acids; (d) a forward primer and a reverse primer comprising or consisting of nucleotide sequences that are complements of the corresponding nucleotide sequences of the forward primer and reverse primer of a primer set selected from the group consisting of (a)-(c); or (e) a combination of a primer set selected from the group consisting of (a)-(d).

In certain embodiments, the oligonucleotide probe is selected from the group consisting of: (a) a probe comprising or consisting of the sequence of SEQ ID NO:17; (b) a probe that has up to three nucleotide changes the sequence of SEQ ID NO:17, wherein the probe is capable of hybridizing to and detecting the PMMoV RNA or amplicon thereof, and (c) a combination of probes selected from the group consisting of (a) and (b).

In certain embodiments, the set of primers and the probe used for detecting the PMMoV in the sample comprises a primer comprising or consisting of the sequence of SEQ ID NO:9, a primer comprising or consisting of the sequence of SEQ ID NO:10, and a probe comprising or consisting of the sequence of SEQ ID NO:17.

In certain embodiments, the method further comprises amplifying the nucleic acids using a set of primers capable of selectively amplifying at least a portion of a SARS-CoV-2 envelope (E) encoding gene and a nucleocapsid protein N2 (N2) encoding gene; detecting the presence of the amplified nucleic acids using a detectably labeled oligonucleotide probe sufficiently complementary to and capable of hybridizing with the SARS-CoV-2 RNA or amplicon thereof, if present, as an indication of the presence or absence of the SARS-CoV-2 in the sample of wastewater; quantifying the amount of the SARS-CoV-2 RNA or amplicon thereof, wherein the amount of the SARS-CoV-2 RNA or amplicon thereof is indicative of the amount of the SARS-CoV-2 in the sample of wastewater; and dividing the amount of the SARS-CoV-2 RNA or amplicon thereof by the amount of the ToBRFV RNA or amplicon thereof, to provide a normalized value for the amount of the SARS-CoV-2 per the amount of human feces in the wastewater.

In certain embodiments, the set of primers for amplifying the SARS-CoV-2 nucleic acids comprises: (a) a forward primer comprising or consisting of the sequence of SEQ ID NO:13 and a reverse primer comprising or consisting of the sequence of SEQ ID NO:14; (b) a forward primer comprising or consisting of the sequence of SEQ ID NO:15 and a reverse primer comprising or consisting of the sequence of SEQ ID NO:16; (c) a forward primer and a reverse primer each comprising at least 10 contiguous nucleotides from the corresponding nucleotide sequences of the forward primer and reverse primer of a primer set selected from the group consisting of (a) and (b); (d) a forward primer and a reverse primer comprising at least one nucleotide sequence that differs from the corresponding nucleotide sequence of the forward primer or reverse primer of a primer set selected from the group consisting of (a)-(c) in that the primer has up to three nucleotide changes compared to the corresponding sequence, wherein the primer is capable of hybridizing to and amplifying the SARS-CoV-2 nucleic acids; (e) a forward primer and a reverse primer comprising or consisting of nucleotide sequences that are complements of the corresponding nucleotide sequences of the forward primer and reverse primer of a primer set selected from the group consisting of (a)-(d); or (f) a combination of a primer set selected from the group consisting of (a)-(e).

In certain embodiments, the oligonucleotide probe is selected from the group consisting of: (a) a probe comprising or consisting of the sequence of SEQ ID NO:19, (b) a probe comprising or consisting of the sequence of SEQ ID NO:20, (c) a probe that differs from the corresponding nucleotide sequence of a probe selected from the group consisting of (a) and (b) in that the probe has up to three nucleotide changes compared to the corresponding sequence, wherein the probe is capable of hybridizing to and detecting the SARS-CoV-2 RNA or amplicon thereof, and (d) a combination of probes selected from the group consisting of (a)-(c).

In certain embodiments, the set of primers and probes used for detecting the SARS-CoV-2 RNA in the sample comprises: a primer comprising or consisting of the sequence of SEQ ID NO:13, a primer comprising or consisting of the sequence of SEQ ID NO:14, and a probe comprising or consisting of the sequence of SEQ ID NO:19; and a primer comprising or consisting of the sequence of SEQ ID NO:15, a primer comprising or consisting of the sequence of SEQ ID NO:16, and a probe comprising or consisting of the sequence of SEQ ID NO:20.

In another aspect, a kit for detecting ToBRFV in a sample is provided, the kit comprising a composition described herein and instructions for detecting ToBRFV.

### BRIEF DESCRIPTION OF THE DRAWINGS

FIG. 1: Relative abundance of viral RNA from sequencing wastewater and stool samples. The x axis represents the source of the eight sequencing data sets analyzed here. Five wastewater samples are marked by the date of collection in YYYYMMDD format followed by “WW” and the location of collection. The three stool samples are marked by the year and timepoint of collection followed by “St” and the location of collection. The y axis indicates the relative abundance of each taxon. The color scheme represents specific taxa as shown in the color key. Patterned bars highlight sequence reads from transcripts from taxa that have DNA genomes. For taxa with 10.0% relative abundance, the percent abundance is also presented in the histogram.

FIGS. 2A-2C. Analysis of newly assembled ToBRFV genomes and generation of primer/probe sets for ddRT-PCR. (FIG. 2A) Phylogenetic tree of 78 nearly complete genomes of ToBRFV, including eight genomes generated in the current study from wastewater and stool. All genomes are listed by their NCBI accession number and source location. Seventy preexisting genomes are listed in black font, five genomes derived from wastewater samples are in yellow, and three from stool samples are in green. (FIG. 2B) Summary of multiple-sequence alignment and gene annotation across the ToBRFV genomes. Green indicates regions that are 100.0% conserved across all genomes, while cream marks those that are greater than 30.0% but less than 100.0% conserved. Two variants of the RdRP-encoding gene are found at 77 bp and are either 4,848 bp or 3,351 bp in size. The Mo protein-encoding gene is found at 4,911 bp and is 801 bp in size. The CP-encoding gene is found at 5,714 bp and is 480 bp in size. Genomic locations are based on the genome ID NC_028478. (FIG. 2C) Sequences of primer/probe sets (SEQ ID NOS:1-6) generated in the current study, in February 2021, aimed at targeting the Mo and RdRP genes across all known genomes. Since the number of known genomes grew from February 2021 to November 2022, the final column indicates the proportion of the 441 current genomes bearing sequences identical to the designed primer/probe sets.

FIG. 3. Concentrations of PMMoV and ToBRFV target genes in animal stool samples. Dot plot marking the concentrations of the PMMoV CP (blue), ToBRFV Mo (yellow) and RdRP (red) genes from the undiluted templates. Concentrations of BCoV M gene, used as a control, are marked by white dots. Error bars marking the standard deviation are plotted along with the dots and are mostly subsumed within the dots. The x axis shows 13 samples from 14 different animals, as a single sample was derived from cohoused ducks and geese. The y axis shows concentrations of the genes. U, undetermined (samples with no detectable gene). * Note that 1:10-diluted RNA template from a pig's stool indicated 7.47 log₁₀ copies/mL of template of the PMMoV CP gene.

FIGS. 4A-4E. Prevalence of PMMoV and ToBRFV target genes in human stool samples. (FIG. 4A) Tabular summary of detection of the three gene targets in samples from adult and pediatric cohorts. The first column lists the name of the target gene and cohort, followed by the number and percentage of samples that were positive (pos) or number of samples that were negative (neg) for that target gene and the total number of samples tested (tot). (FIG. 4B) Venn diagram summarizing the detection of the PMMoV CP gene (blue) and the ToBRFV Mo (yellow) and RdRP (red) genes across 220 human stool samples. In 34 (15.5%), we detected only the PMMoV CP gene, while 13 (5.9%) had only the ToBRFV Mo gene. In 38 (17.3%) samples, we detected both ToBRFV target genes, while 22 (10.0%) had both the PMMoV CP gene and ToBRFV Mo gene. In 70 (31.8%) samples, we detected all three gene targets, while 43 (19.6%) had none of them. (FIG. 4C) Dot plot marking the concentrations of PMMoV CP (blue), ToBRFV Mo (red) and RdRP (yellow) genes, with violin and box plots summarizing their distributions, in RNA extracted from stool samples collected from humans. The x axis marks the target genes, and the y axis shows their concentrations. U, undetermined (samples with no detectable gene target above the LoB). The concentration of the PMMoV CP gene had a median of 1.13 with a standard deviation of 1.00 and IQR of 1.74 log₁₀ copies/mL of template, the ToBRFV Mo gene had a median of 2.12 with a standard deviation of 1.69 and IQR of 2.67 log₁₀ copies/mL of template, and the ToBRFV RdRP gene has a median of 2.20 with a standard deviation of 1.56 and IQR of 2.72 log₁₀ copies/mL of template. P values derived from paired Wilcoxon signed-rank tests with continuity correction and excluding samples with undetermined concentration across all combinations of the three gene targets are listed at the top of the plot. (FIG. 4D) Dot plot marking the relative abundance of viral reads of PMMoV (blue) and ToBRFV (yellow) from previously published metatranscriptomics data derived from healthy stool samples. The x axis shows the 10 donors who provided samples, and each sample provided RNA sequences in biological triplicate; each dot denotes a single replicate. The y axis shows relative abundance. (FIG. 4E) Dot plot summarizing data from FIG. 4D, now including violin and box plots to highlight distribution of viral RNA concentrations and associated statistics. The x axis marks the target viral RNA, and the y axis shows their relative abundance in percent. Dots represent the averages of data from three biological replicates. PMMoV (blue) is present at a median relative abundance of 0.217% with a standard deviation of 9.83% and IQR of 5.19%, ToBRFV (yellow) is present at a median relative abundance of 46.7 with a standard deviation of 48.5% and IQR of 95.4%. The P value at the top was derived from a Wilcoxon signed-rank test of pairwise differences in relative abundance with continuity correction and excluding samples with undetermined concentration.

FIGS. 5A-5B. Concentrations of PMMoV and ToBRFV target genes in wastewater samples from across the USA (FIG. 5A) Dot plot marking the concentrations of PMMoV CP (blue), ToBRFV Mo (yellow), and ToBRFV RDRP (red) genes across samples. Error bars marking the standard deviation are plotted along with the dots and are mostly subsumed within the dots. The x axis shows the 15 cities from which samples were obtained, in decreasing concentration of the Mo gene; abbreviations for states and cities are expanded in Table S2. The y axis shows concentrations of the genes. (FIG. 5B) Dot plot marking the concentrations of PMMoV CP (blue), ToBRFV Mo (red), and RdRP (yellow) genes, with violin and box plots summarizing their distributions, in RNA extracted from wastewater samples collected from across the United States. The x axis marks the target genes, and the y axis shows their concentrations. The PMMoV CP gene has a median of 9.49 with a standard deviation of 0.46 and IQR of 0.74 log₁₀ copies/g (dry weight) of wastewater sample, ToBRFV Mo gene has a median of 10.5 with a standard deviation of 0.67 and IQR of 0.26 log₁₀ copies/g, and ToBRFV RdRP gene has a median of 9.81 with a standard deviation of 0.60 and IQR of 0.36 log₁₀ copies/g. P values were derived from paired Wilcoxon signed-rank tests with continuity correction across all combinations of the three gene targets. U, undetermined (samples with no detectable gene target above the LoB).

FIG. 6. Prevalence of PMMoV, ToBRFV, and crAssphage target genes in stormwater samples from across California. UpSet plot summarizing the number of stormwater samples (total n=9) that are either positive for multiple marker genes (left) or negative for all marker genes (right) in the vertical bar plots. Marker genes are listed under the plots, with colored dots representing presence and gray dots representing absence. Marker genes present in samples represented in the vertical bar are also connected by a thick line. The prevalence of independent marker genes is also summarized in the horizontal bar plot. All bars present data as percentages. Blue, PMMoV CP gene; yellow and red, ToBRFV Mo and RdRP genes, respectively; white, crAssphage ORF000024. Data for crAssphage were derived from a previous study (22).

FIG. 7. Timing of stool collection from human participants undergoing treatment for hematological disorders. The timeline from the start of the study in November 2019 to November 2020 is plotted on the x-axis. Every participant in the cohort is represented by an anonymized study ID on the y-axis and a corresponding horizontal line across the plot. 125 adult participants are represented in the upper panel and 4 pediatric participants are represented in the lower panel. Dates of stool collected are marked by a yellow square.

FIGS. 8A-8B. Summary demographics of participants undergoing treatment for hematological disorders who provided stool samples for this study. (FIG. 8A) Age distribution of 124 adult participants, 79 male and 45 female. The x-axis lists the number of participants and the y-axis lists age ranges at 10-year intervals. The number of participants in each category (range=5 years) is listed at the head of the relevant bars. This data does not include 1 adult participant who did not provide their age and 4 pediatric participants whose ages are not listed to preserve anonymity. (FIG. 8B) Racial and ethnic distribution of 129 adult and pediatric participants. The x-axis lists race or ethnicity categories and the y-axis lists the percentage of participants. The percentage of participants in each category is listed at the top of the relevant bars. Not reported refers to data that is aggregated in order to avoid information that can be used to identify participants. Associated cumulative data are summarized in Table S1.

FIG. 9. Phylogenetic tree of 441 near complete genomes of ToBRFV, including eight genomes generated in the current study from wastewater and stool. 433 preexisting genomes are represented by black, five genomes derived from wastewater samples by yellow, and three by green branches and corresponding dots in the outermost ring. The outer circle highlights the geographic location of the source of the genomes, where blue marks samples from Netherlands, salmon from Mediterranean countries (Cyprus and Greece), cream from Americas (Canada, Peru and U.S.A.), orange from Asia (China and Jordan), and brown from Europe except Netherlands (Belgium, France, Germany, Italy, Switzerland, and United Kingdom).

FIGS. 10A-10F. Analysis of specificity and sensitivity of primer/probes targeting genes in PMMoV and ToBRFV. The x-axis lists the theoretical concentrations of a synthetic plasmid bearing the PMMoV CP (FIGS. 10A and 10B), ToBRFV Mo (FIGS. 10C and 10D), and RdRP (FIGS. 10E and 10F) genes as standards, paired with matched primer/probes in log₁₀ copies/μl, and lists negative samples without the matched target gene. The y-axis lists the gene target concentrations detected using ddRT-PCR in log₁₀ copies/μL of template. Results from three replicates along with their corresponding standard deviation are plotted. The limit of blank (LoB) for each primer/probe combination is marked with a red horizontal line and the corresponding concentrations in log₁₀ copies/μL of template are listed above the line. FIGS. 10A, 10C, and 10E represent the raw data from the standard curves. FIGS. 10B, 10D, and 10F are plotted after applying the LoB to the raw data, and include the linear regression in black and 95.0% confidence interval in gray. Samples that did not amplify are listed as U for undetermined and not included in the linear regression analysis.

FIG. 11. Prevalence PMMoV and ToBRFV target genes in human stool samples detected by ddRT-PCR. Bar plot summarizing the number of human stool samples containing each of the three target genes. The x-axis marks the target genes and the y-axis lists the number of positive samples. 126/220 (57.3%) samples had the PMMoV CP gene (blue), 143/220 (65.0%) had the ToBRFV Mo (yellow) and 106/220 (48.2%) the RdRP (red) genes respectively.

FIGS. 12A-12C. Concentrations of PMMoV and ToBRFV target genes in human stool samples detected by ddRT-PCR. Dot plot marking the concentrations of PMMoV CP (blue), ToBRFV Mo (red) and RdRP (yellow) genes, with violin and box plots summarizing their distributions, in RNA extracted from stool samples collected from humans. The x-axis marks the target genes, and the y-axis lists their in log₁₀ copies/μl of template; U stands for “Undetermined” and marks samples with no detectable gene target above LoB. (FIG. 12A) Concentrations of gene targets from samples derived from adults separated by their treatment cohort with those on HCT on the left and CAR-T on the right. In the HCT cohort, the PMMoV CP gene has a median of 0.970 with a standard deviation of 0.886 and IQR of 1.17 log₁₀ copies/μL of template, TOBRFV Mo gene has a median of 2.16 with a standard deviation of 1.68 and IQR of 2.65 log₁₀ copies/μL of template, and ToBRFV RdRP gene has a median of 2.26 with a standard deviation of 1.58 and IQR of 2.69 log₁₀ copies/μL of template. In the CAR-T cohort, the PMMoV CP gene has a median of 1.18 with a standard deviation of 1.07 and IQR of 1.56 log₁₀ copies/μL of template, ToBRFV Mo gene has a median of 2.02 with a standard deviation of 1.89 and IQR of 3.68 log₁₀ copies/μL of template, and ToBRFV RdRP gene has a median of 1.81 with a standard deviation of 1.72 and IQR of 3.07 log₁₀ copies/μL of template. (FIG. 12B) Pairwise analyses of gene target concentrations from the same samples, with adult samples marked by unfilled circles and pediatric samples marked by a filled diamond. Each panel captures analysis from one pair of gene targets, with concentrations derived from the same RNA extract connected by a line. (FIG. 12C) Concentrations of gene targets derived from adult (unfilled circles) and pediatric (filled diamonds) samples expressed in copies/g dry weight of stool. The PMMoV CP gene has a median of 5.36 with a standard deviation of 0.986 and IQR of 1.74 log₁₀ copies/g dry weight, ToBRFV Mo gene has a median of 6.32 with a standard deviation of 1.69 and IQR of 2.68 log₁₀ copies/g dry weight, and ToBRFV RdRP gene has a median of 6.45 with a standard deviation of 1.57 and IQR of 2.72 log₁₀ copies/g dry weight. p values derived from paired Wilcoxon signed-rank tests with continuity correction and excluding samples with undetermined concentration, across all combinations of the three gene targets are listed at the top of the plot.

FIGS. 13A-13B. Concentrations of PMMoV, ToBRFV and crAssphage target genes in stormwater samples from across California. (FIG. 13A) Dot plot marking the concentrations of the PMMoV CP gene (blue), ToBRFV Mo (yellow) and RdRP (red) genes, and crAssphage ORF000024 (white). Data points have error bars marking the associated standard deviation. Data regarding crAssphage concentration is derived from a previous study (1) and listed without error bars. The x-axis lists the nine stormwater sources from where samples were acquired followed by sample ID, in decreasing concentration of crAssphage RNA. (FIG. 13B) Dot plot summarizing the concentrations of PMMoV CP (blue), ToBRFV Mo (red), RdRP (yellow) genes, and crAssphage ORF000024 (white) from RNA extracted from stormwater samples, with violin and box plots marking their distributions. The x-axis marks the target genes. The PMMoV CP gene has a median of 3.02 with a standard deviation of 0.54 and IQR of 0.44 log₁₀ copies/liter, ToBRFV Mo gene has a median of 3.34 with a standard deviation of 0.98 and IQR of 1.36 log₁₀ copies/liter, ToBRFV RdRP gene has a median of 3.48 with a standard deviation of 0.97 and IQR of 1.24 log₁₀ copies/liter, and crAssphage ORF000024 has a median of 4.65 with a standard deviation of 0.56 and IQR of 0.66 log₁₀ copies/liter. p values derived from paired Wilcoxon signed-rank tests with continuity correction and excluding samples with undetermined concentrations across all combinations of the four gene targets are listed at the top of the plot. The y-axis lists concentrations of the genes in log₁₀ copies/liter of stormwater sample. U stands for “Undetermined” and marks samples with no detectable gene target above LoB.

FIGS. 14A-14K. 1D amplitude of ddRT-PCR assays testing compatibility of primer/probes for multiplexed assays. ddRT-PCR enables the simultaneous detection of two target genes across orthogonal detection channels, one that detects the FAM fluor and the other that detects the HEX fluor. Combinations of the BCoV M gene in channel 1 and SARS-CoV-2 E gene in channel 2 (FIG. 14A), BCoV M gene in channel 1 and SARS-CoV-2 N2 gene in channel 2 (FIG. 14B), BCoV M gene in channel 1 and PMMoV CP gene in channel 2 (FIG. 14C), SARS-CoV-2 E gene in channel 1 and PMMoV CP gene in channel 2 (FIG. 14D), SARS-CoV-2 N1 gene in channel 1 and SARS-CoV-2 E gene in channel 2 (FIG. 14E), SARS-CoV-2 N1 gene in channel 1 and SARS-CoV-N2 E gene in channel 2 (FIG. 14F), SARS-CoV-2 N1 gene in channel 1 and PMMoV CP gene in channel 2 (FIG. 14G), SARS-CoV-2 N2 gene in channel 1 and PMMoV CP gene in channel 2 (FIG. 14H), PMMoV CP gene in channel 1 and SARS-CoV-2 E gene in channel 2 (FIG. 14I), PMMoV CP gene in channel 1 and SARS-CoV-2 N2 gene in channel 2 (FIG. 14J), SARS-CoV-2 RdRP gene in channel 1 and PMMoV CP gene in channel 2 (FIG. 14K) were evaluated. The x-axis lists relevant sample names as water for the no template control, COVID +ve participant for RNA extracted from a COVID +ve participant admitted to the ICU, SARS-CoV-2 RNA for the synthetic SARS-CoV-2 RNA from ATCC, BCoV for the RNA extracted from attenuated BCoV vaccine. The y-axis lists the amplitude of fluorescence in the respective detection channel. The panels on the left correspond to channel 1 detecting the FAM fluor, and on the right correspond to channel 2 detecting the HEX fluor. In each amplitude plot, where relevant, droplets bearing a positive signal are labeled on the right as +ve, the threshold amplitude is labeled as Th, and droplets bearing a negative signal are labeled as −ve. Raw data are presented in Table S6.

### DETAILED DESCRIPTION OF THE INVENTION

Compositions and methods are provided for detecting the tomato brown rugose fruit virus (ToBRFV), which is highly abundant in human stool and wastewater. In particular, methods of using ToBRFV in microbial source tracking as a marker of human fecal contamination are disclosed. Methods of using ToBRFV in wastewater surveillance of pathogens as a marker to quantitate feces are also provided. The methods utilize primers and probes capable of amplifying and/or detecting target ToBRFV sequences in samples suspected of having human fecal contamination.

Before the present compositions and methods are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a virus” includes a plurality of such viruses and reference to “the probe” includes reference to one or more probes and equivalents thereof, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

As used herein, the term “tomato brown rugose fruit virus” or “ToBRFV” refers to a plant virus in the genus Tobamovirus of the family Virgaviridae. The ToBRFV genome is a single-stranded, positive-sense RNA of approximately 6.4 kb, encoding a coat protein, movement protein, and RNA-dependent RNA polymerase. The viral genomic RNA is encapsidated into virions that are rod-shaped and about 300 nm long and 18 nm in diameter. The term ToBRFV includes strains of any clade or subtype, including isolates from the Netherlands (e.g., 38886230, 39962442, and 39941668), Belgium (e.g., GBVC ToBRFV 01 and 02) the UK (TBRFV.21930919), and North American isolates (CA18-01, Ca1A, Ca1B, Ca2, and TBRFV-MX-CP) from the USA, Canada, and Mexico, as well as other strains of ToBRFV that are found in human feces.

“Substantially purified” generally refers to isolation of a substance (compound, polynucleotide, oligonucleotide, protein, or polypeptide) such that the substance comprises the majority percent of the sample in which it resides. Typically in a sample, a substantially purified component comprises 50%, preferably 80%-85%, more preferably 90-95% of the sample. Techniques for purifying polynucleotides oligonucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro-molecules of the same type. The term “isolated” with respect to a polynucleotide or oligonucleotide is a nucleic acid molecule devoid, in whole or part, of sequences normally associated with it in nature; or a sequence, as it exists in nature, but having heterologous sequences in association therewith; or a molecule disassociated from the chromosome.

“Homology” refers to the percent identity between two polynucleotide or two polypeptide moieties. Two nucleic acid, or two polypeptide sequences are “substantially homologous” to each other when the sequences exhibit at least about 50% sequence identity, preferably at least about 75% sequence identity, more preferably at least about 80%-85% sequence identity, more preferably at least about 90% sequence identity, and most preferably at least about 95%-98% sequence identity over a defined length of the molecules. As used herein, substantially homologous also refers to sequences showing complete identity to the specified sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353-358, National biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, peptide nucleic acids (PNAs), morpholino nucleic acids, locked nucleic acids (LNAs), glycol nucleic acids (GNAs), threose nucleic acids (TNAs) and hexitol nucleic acids (HNAs). and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms for include, example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

A ToBRFV virus polynucleotide, oligonucleotide, nucleic acid and nucleic acid molecule, as defined above, is a nucleic acid molecule derived from ToBRFV, including, without limitation, any of the various ToBRFV strains found in human feces. The molecule need not be physically derived from the particular isolate in question, but may be synthetically or recombinantly produced.

Nucleic acid sequences for a number of ToBRFV isolates are known. A representative ToBRFV sequence is presented in SEQ ID NO:8 of the Sequence Listing. Additional representative sequences, including sequences of the RNA dependent RNA polymerase (RdRP) encoding gene, the movement protein (Mo) encoding gene, and the coat protein (CP) encoding gene from ToBRFV isolates are listed in the National Center for Biotechnology Information (NCBI) database. See, for example, NCBI entries: Accession No. NC_028478, OR225613, OK358628, MZ542763, OR593752, MZ945420, MZ945419, OR792460, OP557566, OR451555, OQ190155, OP967027, OP967026, OP967025, OP967024, OP967023, OP967022, OQ674195, OQ674194, OM305070, MT018320, MN815773, MK109003, MK109002, MK648157, OP557568, OM515272, OM515271, OM515270, OM515269, OM515268, OM515265, OM515261, OM515260, OM515259, OM515258, OM515250, OM515245, OM515242, OM515241, OM515240, OM515236, OM515235, OM515232, OM515231, MW349655, MZ323110, MW314137, MN882062, MN882061, MN882060, MN882059, MN882050, MN882049, MN882041, MN882040, MN882017, MN882016, MN882058, MN882057, MT002973, MN549394, MN549397, MN549395, and MN549396; all of which sequences (as entered by the date of filing of this application) are herein incorporated by reference. See also Zhang et al. (2022) Mol. Plant Pathol. 23(9):1262-1277, Abrahamian et al. (2022) Viruses 14(12):2816, and van de Vossenberg et al. (2020) PLoS One, 15, e0234671 for sequence comparisons and a discussion of genetic diversity and phylogenetic analysis of ToBRFV.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

As used herein, a “solid support” refers to a solid surface such as a magnetic bead, latex bead, microtiter plate well, glass plate, nylon, agarose, acrylamide, and the like.

As used herein, the term “target nucleic acid region” or “target nucleic acid” denotes a nucleic acid molecule with a “target sequence” to be amplified. The target nucleic acid may be either single-stranded or double-stranded and may include other sequences besides the target sequence, which may not be amplified. The term “target sequence” refers to the particular nucleotide sequence of the target nucleic acid which is to be amplified. The target sequence may include a probe-hybridizing region contained within the target molecule with which a probe will form a stable hybrid under desired conditions. The “target sequence” may also include the complexing sequences to which the oligonucleotide primers complex and are extended using the target sequence as a template. Where the target nucleic acid is originally single-stranded, the term “target sequence” also refers to the sequence complementary to the “target sequence” as present in the target nucleic acid. If the “target nucleic acid” is originally double-stranded, the term “target sequence” refers to both the plus (+) and minus (−) strands (or sense and anti-sense strands).

The term “primer” or “oligonucleotide primer” as used herein, refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is preferably single-stranded for maximum efficiency in amplification, but may alternatively be double-stranded. If double-stranded, the primer can first be treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis. Typically, ToBRFV nucleic acids are amplified using at least one set of oligonucleotide primers comprising at least one forward primer and at least one reverse primer capable of hybridizing to regions of a ToBRFV nucleic acid flanking the portion of the ToBRFV nucleic acid to be amplified. A forward primer is complementary to the 3′ end of the antigenomic ToBRFV template produced during replication or amplification of ToBRFV nucleic acids. A reverse primer is complementary to the 3′ end of the ToBRFV positive sense genomic RNA strand.

The term “amplicon” refers to the amplified nucleic acid product of a PCR reaction or other nucleic acid amplification process (e.g., rolling circle amplification or isothermal amplification methods such as recombinase polymerase amplification (RPA), loop-mediated isothermal amplification (LAMP), strand displacement amplification (SDA), helicase-dependent amplification (HDA), nicking enzyme amplification reaction (NEAR), ligase chain reaction (LGR), nucleic acid sequence based amplification (NASBA), transcription-mediated amplification (TMA), Q-beta amplification, and the like. Amplicons may comprise RNA or DNA depending on the technique used for amplification. For example, DNA amplicons may be generated by RT-PCR, whereas RNA amplicons may be generated by TMA/NASBA.

As used herein, the term “probe” or “oligonucleotide probe” refers to a polynucleotide, as defined above, that contains a nucleic acid sequence complementary to a nucleic acid sequence present in the target nucleic acid analyte. The polynucleotide regions of probes may be composed of DNA, and/or RNA, and/or synthetic nucleotide analogs. Probes may be labeled in order to detect the target sequence. Such a label may be present at the 5′ end, at the 3′ end, at both the 5′ and 3′ ends, and/or internally. The “oligonucleotide probe” may contain at least one fluorescer and at least one quencher. Quenching of fluorophore fluorescence may be eliminated by exonuclease cleavage of the fluorophore from the oligonucleotide (e.g., TaqMan assay) or by hybridization of the oligonucleotide probe to the nucleic acid target sequence (e.g., molecular beacons). Additionally, the oligonucleotide probe will typically be derived from a sequence that lies between the sense and the antisense primers when used in a nucleic acid amplification assay.

As used herein, the term “capture oligonucleotide” refers to an oligonucleotide that contains a nucleic acid sequence complementary to a nucleic acid sequence present in the target nucleic acid analyte such that the capture oligonucleotide can “capture” the target nucleic acid. One or more capture oligonucleotides can be used in order to capture the target analyte. The polynucleotide regions of a capture oligonucleotide may be composed of DNA, and/or RNA, and/or synthetic nucleotide analogs. By “capture” is meant that the analyte can be separated from other components of the sample by virtue of the binding of the capture molecule to the analyte. Typically, the capture molecule is associated with a solid support, either directly or indirectly.

It will be appreciated that the hybridizing sequences need not have perfect complementarity to provide stable hybrids. In many situations, stable hybrids will form where fewer than about 10% of the bases are mismatches, ignoring loops of four or more nucleotides. Accordingly, as used herein the term “complementary” refers to an oligonucleotide that forms a stable duplex with its “complement” under assay conditions, generally where there is about 90% or greater homology.

The terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis.

As used herein, the term “binding pair” refers to first and second molecules that specifically bind to each other, such as complementary polynucleotide pairs capable of forming nucleic acid duplexes. “Specific binding” of the first member of the binding pair to the second member of the binding pair in a sample is evidenced by the binding of the first member to the second member, or vice versa, with greater affinity and specificity than to other components in the sample. The binding between the members of the binding pair is typically noncovalent. Unless the context clearly indicates otherwise, the terms “affinity molecule” and “target analyte” are used herein to refer to first and second members of a binding pair, respectively.

The terms “specific-binding molecule” and “affinity molecule” are used interchangeably herein and refer to a molecule that will selectively bind, through chemical or physical means to a detectable substance present in a sample. By “selectively bind” is meant that the molecule binds preferentially to the target of interest or binds with greater affinity to the target than to other molecules. For example, a DNA molecule will bind to a substantially complementary sequence and not to unrelated sequences. An oligonucleotide that “specifically binds” to a particular type of ToBRFV, such as a particular strain of ToBRFV, denotes an oligonucleotide, e.g., a primer, probe or a capture oligonucleotide, that binds to the particular ToBRFV strain, but does not bind to a sequence from other types of ToBRFVs.

The terms “selectively detects” or “selectively detecting” refer to the detection of ToBRFV nucleic acids using oligonucleotides, e.g., primers, probes and/or capture oligonucleotides that are capable of detecting a particular ToBRFV nucleic acid, for example, by amplifying and/or binding to at least a portion of an RNA segment from a particular type of ToBRFV, such as a particular ToBRFV strain, but do not amplify and/or bind to sequences from other types of ToBRFV under appropriate hybridization conditions.

The “melting temperature” or “Tₘ” of double-stranded DNA is defined as the temperature at which half of the helical structure of DNA is lost due to heating or other dissociation of the hydrogen bonding between base pairs, for example, by acid or alkali treatment, or the like. The Tₘ of a DNA molecule depends on its length and on its base composition. DNA molecules rich in GC base pairs have a higher Tₘ than those having an abundance of AT base pairs. Separated complementary strands of DNA spontaneously reassociate or anneal to form duplex DNA when the temperature is lowered below the Tₘ. The highest rate of nucleic acid hybridization occurs approximately 25° C. below the Tₘ. The Tₘ may be estimated using the following relationship: Tₘ=69.3±0.41(GC) % (Marmur et al. (1962) J. Mol. Biol. 5:109-118).

As used herein, a “sample” refers to a sample being tested for or suspected of containing human fecal contamination. The term “sample” includes stool samples, environmental samples, and fomite samples. Samples may include water samples such as, but not limited to, samples of wastewater, stormwater, ocean water, lake water, river water, creek water, drinking water, recreational water, ground water, source water, stored water, seepage water, surface water, or water from a water distribution system or sewage and waste water treatment system; samples of air potentially containing aerosolized stool matter; earth samples such as, but not limited to, samples of soil, sand, mud, sediment, or rock; sludge samples; or samples from surfaces such as a food preparation surfaces, including, but not limited to, counters, cutting boards, tableware, utensils, measuring cups/spoons, spatulas, dishware, glassware, cutlery, pots and pans, cooking equipment, or tables; or samples from fomite surfaces such as, but not limited to, samples from surfaces of clothes, bedding, utensils, cups, furniture, vehicles, shovels, bowls/buckets, brushes, tack, clippers, pencils, bath faucet handles, toilet flush levers, door knobs, light switches, handrails, elevator buttons, television remote controls, pens, touch screens, common-use phones, keyboards and computer mice, coffeepot handles, countertops, drinking fountains, medical equipment such as, but not limited to, stethoscopes, intravenous (IV) drip tubes, catheters, and life support equipment; and any other items that are frequently touched by different people and infrequently cleaned. The definition also includes samples that have been manipulated in any way after their procurement, such as by treatment with reagents, washed, filtered, concentrated, or enriched for ToBRFV or particular nucleic acids (e.g., ToBRFV RNA).

As used herein, the terms “label” and “detectable label” refer to a molecule capable of detection, including, but not limited to, radioactive isotopes, fluorescers, chemiluminescers, chromophores, enzymes (e.g., horseradish peroxidase (HRP) and α-β-galactosidase), enzyme substrates, enzyme cofactors, enzyme inhibitors, semiconductor nanoparticles, dyes, metal ions, metal sols, ligands (e.g., biotin, strepavidin or haptens) and the like. The term “fluorescer” refers to a substance or a portion thereof which is capable of exhibiting fluorescence in a detectable range. Particular examples of labels which may be used in the practice of the invention include, but are not limited to, fluorophores such as SYBR® green, SYBR® gold, a CAL Fluor® dye such as CAL Fluor® Gold 540, CAL Fluor® Orange 560, CAL Fluor® Red 590, CAL Fluor® Red 610, and CAL Fluor® Red 635, a Quasar® dye such as Quasar® 570, Quasar® 670, and Quasar® 705, an Alexa Fluor® dye such as Alexa Fluor® 488, Alexa Fluor® 546, Alexa Fluor® 555, Alexa Fluor® 594, Alexa Fluor® 647, Alexa Fluor® 660, Alexa Fluor® 700, Alexa Fluor® 750, and Alexa Fluor® 784, and Alexa Fluor® 790, a cyanine dye such as Cy3, Cy3.5, Cy5, Cy5.5, Cy7, and Cy7.5, fluorescein, 2′, 4′, 5′, 7′-tetrachloro-4-7-dichlorofluorescein (TET), carboxyfluorescein (FAM), fluorescein isothiocyanate (FITC), 6-carboxy-4′,5′-dichloro-2′,7′-dimethoxyfluorescein (JOE), hexachlorofluorescein (HEX), rhodamine, carboxy-X-rhodamine (ROX), tetramethyl rhodamine (TAMRA), 5,6-carboxyrhodamine-110 (R110), 6-carboxyrhodamine-6G (R6G), Texas Red, Yakima Yellow, Dragonfly orange, IRDye® dyes such as IRDye® 800CW, IRDye® 680RD, IRDye® 700, IRDye® 750, and IRDye® 800RS, CFR dyes such as CF680, CF680R, CF750, CF770, and CF790, Tracy® dyes such as Tracy® 645 and Tracy® 652), thienothiadiazole dyes, phthalocyanine dyes, squaraine dyes, Si-pyronine, Si-rhodamine, Te-rhodamine, Changsha, borondipyrromethane (BODIPY) dyes, seminaphthofluorone xanthene dyes, benzo[c]heterocycle dyes (e.g., isobenzofuran dyes), and quantum dots.

By “quencher” is meant a substance that is capable of absorbing energy from an excited fluorophore when the quencher is located in close proximity to the fluorophore thereby suppressing its emission of fluorescence. A quencher may dissipate the energy absorbed from the fluorophore, for example, as either heat (in the case of dark quenchers) or visible light (in the case of fluorescent quenchers). When the fluorophore and the quencher are separated by a great enough distance such that the quencher can no longer absorb the fluorescent emission, the fluorescence from the fluorophore can be detected. Exemplary quenchers include, without limitation, black hole quenchers such as Black Hole Quencher®-0 (BHQ-0), Black Hole Quencher®-1 (BHQ-1), Black Hole Quencher®-2 (BHQ-2), and Black Hole Quencher®-3 (BHQ-3), BHQplus®, and BHQnova®; BlackBerry™ Quenchers such as BBQ-650; Eclipse® Quencher, ATTO 540Q, ATTO 575Q, ATTO 580Q, and ATTO 612Q, and MB2, TAMRA, and dabcyl. Probes may be labeled with a quencher and fluorophore having overlapping absorption and emission spectra, respectively, which are incorporated into the probe as a pair. A probe can be designed such that the quencher and fluorophore remain in close proximity if a specific target sequence is not present, and widely separated if it is present, wherein detection of a fluorescent signal indicates the presence of the target sequence, and lack of a fluorescent signal indicates absence of the target sequence.

A “molecular beacon” probe is a single-stranded oligonucleotide, typically 25 to 40 bases-long, in which the bases on the 3′ and 5′ ends are complementary forming a “stem,” typically for 5 to 8 base pairs. A molecular beacon probe forms a hairpin structure at temperatures at and below those used to anneal the primers to the template (typically below about 60° C.). The double-helical stem of the hairpin brings a fluorophore (or other label) attached to the 5′ end of the probe in proximity to a quencher attached to the 3′ end of the probe. The probe does not fluoresce (or otherwise provide a signal) in this conformation. If a probe is heated above the temperature needed to melt the double stranded stem apart, or the probe hybridizes to a target nucleic acid that is complementary to the sequence within the single-strand loop of the probe, the fluorophore and the quencher are separated, and the fluorophore fluoresces in the resulting conformation. Therefore, in a series of PCR cycles the strength of the fluorescent signal increases in proportion to the amount of the molecular beacon that is hybridized to the amplicon, when the signal is read at the annealing temperature. Molecular beacons of high specificity, having different loop sequences and conjugated to different fluorophores, can be selected in order to monitor increases in amplicons that differ by as little as one base (Tyagi, S. and Kramer, F. R. (1996), Nat. Biotech. 14:303 308; Tyagi, S. et al., (1998), Nat. Biotech. 16: 49 53; Kostrikis, L. G. et al., (1998), Science 279: 1228 1229; all of which are herein incorporated by reference).

By “subject” is meant any member of the subphylum Chordata, including, without limitation, humans and other primates, including non-human primates such as chimpanzees and other apes and monkey species; farm animals such as cattle, sheep, pigs, goats and horses; domestic mammals such as dogs and cats; birds; and laboratory animals, including rodents such as mice, rats and guinea pigs, and the like. The term does not denote a particular age. Thus, both adult and newborn individuals are intended to be covered.

### Microbial Source Tracking Using ToBRFV as a Marker of Human Fecal Contamination

Compositions and methods of using ToBRFV in microbial source tracking as a marker of human fecal contamination are disclosed. The subject methods are useful for detecting and quantitating human fecal contamination based on the presence of ToBRFV RNA in a sample. The methods utilize primers and probes capable of amplifying and/or detecting target ToBRFV nucleic acid sequences in samples suspected of having human fecal contamination.

ToBRFV can be used to detect human fecal contamination, for example, in stool samples and environmental samples and on fomites. Samples that can be tested for human fecal contamination, according to the methods disclosed herein, include, without limitation, water samples such as, but not limited to, samples of wastewater, stormwater, ocean water, lake water, river water, creek water, drinking water, recreational water, ground water, source water, stored water, seepage water, surface water, or water from a water distribution system or sewage and waste water treatment system; samples of air potentially containing aerosolized stool matter; earth samples such as, but not limited to, samples of soil, sand, mud, sediment, or rock; sludge samples; and samples from surfaces such as a food preparation surfaces, including, but not limited to, counters, cutting boards, tableware, utensils, measuring cups/spoons, spatulas, dishware, glassware, cutlery, pots and pans, cooking equipment, or tables; or samples from fomite surfaces such as, but not limited to, clothes, bedding, utensils, cups, furniture, vehicles, shovels, bowls/buckets, brushes, tack, clippers, pencils, bath faucet handles, toilet flush levers, door knobs, light switches, handrails, elevator buttons, television remote controls, pens, touch screens, common-use phones, keyboards and computer mice, coffeepot handles, countertops, drinking fountains, medical equipment such as, but not limited to, stethoscopes, intravenous (IV) drip tubes, catheters, and life support equipment; and any other items that are frequently touched by different people and infrequently cleaned. Samples may be manipulated in any way after their procurement, such as by treatment with reagents, washed, filtered, concentrated, or enriched for ToBRFV or particular nucleic acids (e.g., ToBRFV RNA).

The methods use oligonucleotide reagents (e.g., oligonucleotide primers and probes) or a combination of reagents capable of detecting one or more strains of ToBRFV in a single assay. In one format, primer pairs and probes capable of detecting one or more strains of ToBRFV are used, In some embodiments, certain primers and probes are from “conserved” regions and therefore capable of detecting more than one strain of ToBRFV. By way of example, the RNA dependent RNA polymerase (RdRP) encoding gene and the movement protein (Mo) encoding gene of ToBRFV include conserved regions. Thus, primers and probes comprising sequences from these conserved regions of the ToBRFV genome may be useful in detecting multiple ToBRFV strains. In some embodiments, the primers and probes comprise sequences from conserved regions of the RdRP encoding gene and/or the Mo encoding gene from the genome of the reference ToBRFV isolate Tom1-Jo, which comprises the genomic sequence of SEQ ID NO:8. In other embodiments, primers and probes comprising sequences from the corresponding conserved regions of the RdRP encoding gene and/or the Mo encoding gene in the genomes of other strains of ToBRFV may be used in detecting multiple ToBRFV strains. Conserved regions of the RdRP encoding gene and the Mo encoding gene can be amplified and detected simultaneously by using a combination of RdRP-specific and Mo-specific primers and probes in a multiplex-type assay format.

Thus, oligonucleotides for use in the assays described herein can be derived from the conserved regions of the RdRP encoding gene and/or the Mo encoding gene from the genome of ToBRFV. Representative sequences from ToBRFV isolates are listed herein. Thus, primers and probes for use in detection of ToBRFV include those derived from any ToBRFV clade, strain, or isolate found in human feces. A representative ToBRFV sequence is presented in SEQ ID NO:8 of the Sequence Listing. Additional representative sequences, including sequences of the RNA dependent RNA polymerase (RdRP) encoding gene, the movement protein (Mo) encoding gene, and the coat protein (CP) encoding gene from ToBRFV isolates are listed in the National Center for Biotechnology Information (NCBI) database. See, for example, NCBI entries: Accession No. NC_028478, OR225613, OK358628, MZ542763, OR593752, MZ945420, MZ945419, OR792460, OP557566, OR451555, OQ190155, OP967027, OP967026, OP967025, OP967024, OP967023, OP967022, OQ674195, OQ674194, OM305070, MT018320, MN815773, MK109003, MK109002, MK648157, OP557568, OM515272, OM515271, OM515270, OM515269, OM515268, OM515265, OM515261, OM515260, OM515259, OM515258, OM515250, OM515245, OM515242, OM515241, OM515240, OM515236, OM515235, OM515232, OM515231, MW349655, MZ323110, MW314137, MN882062, MN882061, MN882060, MN882059, MN882050, MN882049, MN882041, MN882040, MN882017, MN882016, MN882058, MN882057, MT002973, MN549394, MN549397, MN549395, and MN549396; all of which sequences (as entered by the date of filing of this application) are herein incorporated by reference. See also Zhang et al. (2022) Mol. Plant Pathol. 23(9):1262-1277, Abrahamian et al. (2022) Viruses 14(12):2816, and van de Vossenberg et al. (2020) PLoS One, 15, e0234671 for sequence comparisons and a discussion of genetic diversity and phylogenetic analysis of ToBRFV.

Primers and probes for use in the assays herein are derived from these sequences and are readily synthesized by standard techniques, e.g., solid phase synthesis via phosphoramidite chemistry, as disclosed in U.S. Pat. Nos. 4,458,066 and 4,415,732, incorporated herein by reference; Beaucage et al., Tetrahedron (1992) 48:2223-2311; and Applied Biosystems User Bulletin No. 13 (1 Apr. 1987). Other chemical synthesis methods include, for example, the phosphotriester method described by Narang et al., Meth. Enzymol. (1979) 68:90 and the phosphodiester method disclosed by Brown et al., Meth. Enzymol. (1979) 68:109. Poly(A) or poly(C), or other non-complementary nucleotide extensions may be incorporated into oligonucleotides using these same methods. Hexaethylene oxide extensions may be coupled to the oligonucleotides by methods known in the art. Cload et al., J. Am. Chem. Soc. (1991) 113:6324-6326; U.S. Pat. No. 4,914,210 to Levenson et al.; Durand et al., Nucleic Acids Res. (1990) 18:6353-6359; and Horn et al., Tet. Lett. (1986) 27:4705-4708.

Alternatively, ToBRFV can be isolated from infected plants such as tomato and pepper plants using standard methods. For example, a ToBRFV isolate can be obtained by grinding infected leaves of plants in phosphate buffer and filtering through cheesecloth to obtain an extract containing the virus. Uninfected plants can be inoculated with the extract and grown in a greenhouse at 24±2° C. with a 14/10 hour photoperiod and 50-70% relative humidity to produce the ToBRFV isolate. See, e.g., Jewehan et al. (2022) Arch Virol. 167(7):1559-1563; herein incorporated by reference.

An amplification method such as PCR or nucleic acid sequence-based amplification (NASBA) can be used to amplify polynucleotides from either ToBRFV genomic RNA or cDNA derived therefrom. Alternatively, polynucleotides can be synthesized in the laboratory, for example, using an automatic synthesizer.

Typically, the primer oligonucleotides are in the range of between 10-100 nucleotides in length, such as 15-60, 20-40 and so on, more typically in the range of between 20-40 nucleotides long, and any length between the stated ranges. In certain embodiments, a primer oligonucleotide comprises or consists of a sequence selected from the group consisting of SEQ ID NO:1, SEQ ID NO:2, SEQ ID NO:4, and SEQ ID NO:5; or a fragment thereof comprising at least about 6 contiguous nucleotides, preferably at least about 8 contiguous nucleotides, more preferably at least about 10-12 contiguous nucleotides, and even more preferably at least about 15-20 contiguous nucleotides; or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto. Changes to the nucleotide sequences of SEQ ID NO:1, SEQ ID NO:2, SEQ ID NO:4, and SEQ ID NO:5 may be introduced corresponding to genetic variations in particular ToBRFV strains. In certain embodiments, up to three nucleotide changes, including 1 nucleotide change, 2 nucleotide changes, or three nucleotide changes, may be made in a sequence selected from the group consisting of SEQ ID NO:1, SEQ ID NO:2, SEQ ID NO:4, and SEQ ID NO:5, wherein the oligonucleotide primer is capable of hybridizing to and amplifying a particular ToBRFV target nucleic acid.

The typical probe oligonucleotide is in the range of between 10-100 nucleotides long, such as 10-60, 15-40, 18-30, and so on, and any length between the stated ranges. In certain embodiments, a probe oligonucleotide comprises or consists of a sequence selected from the group consisting of SEQ ID NO:3, SEQ ID NO:6, and SEQ ID NO:7; or a fragment thereof comprising at least about 6 contiguous nucleotides, preferably at least about 8 contiguous nucleotides, more preferably at least about 10-12 contiguous nucleotides, and even more preferably at least about 15-20 contiguous nucleotides; or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto. Changes to the nucleotide sequences of SEQ ID NO:3, SEQ ID NO:6, and SEQ ID NO:7 may be introduced corresponding to genetic variations in particular ToBRFV strains. In certain embodiments, up to three nucleotide changes, including 1 nucleotide change, 2 nucleotide changes, or three nucleotide changes, may be made in a sequence selected from the group consisting of SEQ ID NO:3 and SEQ ID NO:6, wherein the oligonucleotide probe is capable of hybridizing to and detecting a particular ToBRFV target nucleic acid.

It is to be understood that the primers and probes described herein are merely representative, and other oligonucleotides derived from various ToBRFV strains will find use in the assays described herein.

Moreover, the oligonucleotides, particularly the probe oligonucleotides, may be coupled to labels for detection. There are several means known for derivatizing oligonucleotides with reactive functionalities which permit the addition of a label. For example, several approaches are available for biotinylating probes so that radioactive, fluorescent, chemiluminescent, enzymatic, or electron dense labels can be attached via avidin. See, e.g., Broken et al., Nucl. Acids Res. (1978) 5:363-384 which discloses the use of ferritin-avidin-biotin labels; and Chollet et al., Nucl. Acids Res. (1985) 13:1529-1541 which discloses biotinylation of the 5′ termini of oligonucleotides via an aminoalkylphosphoramide linker arm. Several methods are also available for synthesizing amino-derivatized oligonucleotides which are readily labeled by fluorescent or other types of compounds derivatized by amino-reactive groups, such as isothiocyanate, N-hydroxysuccinimide, or the like, see, e.g., Connolly, Nucl. Acids Res. (1987) 15:3131-3139, Gibson et al. Nucl. Acids Res. (1987) 15:6455-6467 and U.S. Pat. No. 4,605,735 to Miyoshi et al. Methods are also available for synthesizing sulfhydryl-derivatized oligonucleotides, which can be reacted with thiol-specific labels, see, e.g., U.S. Pat. No. 4,757,141 to Fung et al., Connolly et al., Nucl. Acids Res. (1985) 13:4485-4502 and Spoat et al. Nucl. Acids Res. (1987) 15:4837-4848. A comprehensive review of methodologies for labeling DNA fragments is provided in Matthews et al., Anal. Biochem. (1988) 169:1-25.

For example, oligonucleotides may be fluorescently labeled by linking a fluorescent molecule to the non-ligating terminus of the molecule. Guidance for selecting appropriate fluorescent labels can be found in Smith et al., Meth. Enzymol. (1987) 155:260-301; Karger et al., Nucl. Acids Res. (1991) 19:4955-4962; Guo et al. (2012) Anal. Bioanal. Chem. 402(10):3115-3125; and Molecular Probes Handbook, A Guide to Fluorescent Probes and Labeling Technologies, 11^{th} edition, Johnson and Spence eds., 2010 (Molecular Probes/Life Technologies). Fluorescent labels include fluorescein and derivatives thereof, such as disclosed in U.S. Pat. No. 4,318,846 and Lee et al., Cytometry (1989) 10:151-164. Exemplary dyes that can be used in the practice of the methods disclosed herein include, but are not limited to, 3-phenyl-7-isocyanatocoumarin, acridines, such as 9-isothiocyanatoacridine and acridine orange, pyrenes, benzoxadiazoles, and stilbenes, such as disclosed in U.S. Pat. No. 4,174,384. Additional fluorescent labels include SYBR® green, SYBR® gold, a CAL Fluor® dye such as CAL Fluor® Gold 540, CAL Fluor® Orange 560, CAL Fluor® Red 590, CAL Fluor® Red 610, and CAL Fluor® Red 635, a Quasar® dye such as Quasar® 570, Quasar® 670, and Quasar® 705, an Alexa Fluor® dye such as Alexa Fluor® 488, Alexa Fluor® 546, Alexa Fluor® 555, Alexa Fluor® 594, Alexa Fluor® 647, Alexa Fluor® 660, Alexa Fluor® 700, Alexa Fluor® 750, and Alexa Fluor® 784, and Alexa Fluor® 790, a cyanine dye such as Cy3, Cy3.5, Cy5, Cy5.5, Cy7, and Cy7.5, fluorescein, 2′, 4′, 5′, 7′-tetrachloro-4-7-dichlorofluorescein (TET), carboxyfluorescein (FAM), fluorescein isothiocyanate (FITC), 6-carboxy-4′,5′-dichloro-2′,7′-dimethoxyfluorescein (JOE), hexachlorofluorescein (HEX), rhodamine, carboxy-X-rhodamine (ROX), tetramethyl rhodamine (TAMRA), 5,6-carboxyrhodamine-110 (R110), 6-carboxyrhodamine-6G (R6G), Texas Red, Yakima Yellow, Dragonfly orange, IRDye® dyes such as IRDye® 800CW, IRDye® 680RD, IRDye® 700, IRDye® 750, and IRDye® 800RS, CF® dyes such as CF680, CF680R, CF750, CF770, and CF790, Tracy® dyes such as Tracy® 645 and Tracy® 652), thienothiadiazole dyes, phthalocyanine dyes, squaraine dyes, Si-pyronine, Si-rhodamine, Te-rhodamine, Changsha, borondipyrromethane (BODIPY) dyes, seminaphthofluorone xanthene dyes, benzo[c]heterocycle dyes (e.g., isobenzofuran dyes), and quantum dots. These fluorophores are commercially available from various suppliers such as Thermo Fisher Scientific (Waltham, MA), LGC Biosearch Technologies (Hoddesdon, United Kingdom), and Integrated DNA Technologies (Coralville, Iowa). When used in a multiplex assay, different probes may be labeled with different dyes so that they can be detected in different detection channels.

Oligonucleotides can also be labeled with a minor groove binding (MGB) molecule, such as disclosed in U.S. Pat. Nos. 6,884,584, 5,801,155; Afonina et al. (2002) Biotechniques 32:940-944, 946-949; Lopez-Andreo et al. (2005) Anal. Biochem. 339:73-82; and Belousov et al. (2004) Hum Genomics 1:209-217. Oligonucleotides having a covalently attached MGB are more sequence specific for their complementary targets than unmodified oligonucleotides. In addition, an MGB group increases hybrid stability with complementary DNA target strands compared to unmodified oligonucleotides, allowing hybridization with shorter oligonucleotides.

Additionally, oligonucleotides can be labeled with an acridinium ester (AE) using the techniques described below. Current technologies allow the AE label to be placed at any location within the probe. See, e.g., Nelson et al., (1995) “Detection of Acridinium Esters by Chemiluminescence” in Nonisotopic Probing, Blotting and Sequencing, Kricka L. J. (ed) Academic Press, San Diego, Calif.; Nelson et al. (1994) “Application of the Hybridization Protection Assay (HPA) to PCR” in The Polymerase Chain Reaction, Mullis et al. (eds.) Birkhauser, Boston, Mass.; Weeks et al., Clin. Chem. (1983) 29:1474-1479; Berry et al., Clin. Chem. (1988) 34:2087-2090. An AE molecule can be directly attached to the probe using non-nucleotide-based linker arm chemistry that allows placement of the label at any location within the probe. See, e.g., U.S. Pat. Nos. 5,585,481 and 5,185,439.

In certain embodiments, molecular beacon probes may be used for detection of ToBRFV target nucleic acids. Molecular beacons are hairpin shaped oligonucleotides with an internally quenched fluorophore. Molecular beacons typically comprise four parts: a loop of about 18-30 nucleotides, which is complementary to the target nucleic acid sequence; a stem formed by two oligonucleotide regions that are complementary to each other, each about 5 to 7 nucleotide residues in length, on either side of the loop; a fluorophore covalently attached to the 5′ end of the molecular beacon, and a quencher covalently attached to the 3′ end of the molecular beacon. When the beacon is in its closed hairpin conformation, the quencher resides in proximity to the fluorophore, which results in quenching of the fluorescent emission from the fluorophore. In the presence of a target nucleic acid having a region that is complementary to the strand in the molecular beacon loop, hybridization occurs resulting in the formation of a duplex between the target nucleic acid and the molecular beacon. Hybridization disrupts intramolecular interactions in the stem of the molecular beacon and causes the fluorophore and the quencher of the molecular beacon to separate resulting in a fluorescent signal from the fluorophore that indicates the presence of the target nucleic acid sequence. See, e.g., Guo et al. (2012) Anal. Bioanal. Chem. 402(10):3115-3125; Wang et al. (2009) Angew. Chem. Int. Ed. Engl. 48(5):856-870; and Li et al. (2008) Biochem. Biophys. Res. Commun. 373(4):457-461; herein incorporated by reference in their entireties.

When utilizing a hybridization-based detection system, a nucleic acid probe is chosen that is complementary to a target nucleic acid sequence. By selection of appropriate conditions, the probe and the target sequence “selectively hybridize,” or bind, to each other to form a hybrid molecule. An oligonucleotide that “selectively hybridizes” to a particular ToBRFV sequence from a particular strain under hybridization conditions described below, denotes an oligonucleotide, e.g., a primer or probe oligonucleotide, that binds to the ToBRFV sequence of that particular ToBRFV strain, but does not bind to a sequence from a ToBRFV of a different strain.

In one embodiment of the present invention, a nucleic acid molecule is capable of hybridizing selectively to a target sequence under moderately stringent hybridization conditions. In the context of the present invention, moderately stringent hybridization conditions allow detection of a target nucleic acid sequence of at least 14 nucleotides in length having at least approximately 70% sequence identity with the sequence of the selected nucleic acid probe. In another embodiment, such selective hybridization is performed under stringent hybridization conditions. Stringent hybridization conditions allow detection of target nucleic acid sequences of at least 14 nucleotides in length having a sequence identity of greater than 90% with the sequence of the selected nucleic acid probe. Hybridization conditions useful for probe/target hybridization where the probe and target have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press). Hybrid molecules can be formed, for example, on a solid support, in solution, and in tissue sections. The formation of hybrids can be monitored by inclusion of a reporter molecule, typically, in the probe. Such reporter molecules or detectable labels include, but are not limited to, radioactive elements, fluorescent markers, and molecules to which an enzyme-conjugated ligand can bind.

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature and time parameters, as well as, varying wash conditions. The selection of a particular set of hybridization conditions is well known (see, for example, Sambrook, et al., Molecular Cloning: A Laboratory Manual, 3rd Edition, 2001).

Any primer-dependent amplification method known in the art may be used for amplification of target ToBRFV nucleic acids including, without limitation, polymerase chain reaction (PCR), rolling circle amplification or isothermal amplification methods such as recombinase polymerase amplification (RPA), loop-mediated isothermal amplification (LAMP), strand displacement amplification (SDA), helicase-dependent amplification (HDA), nicking enzyme amplification reaction (NEAR), ligase chain reaction (LGR), nucleic acid sequence based amplification (NASBA), transcription-mediated amplification (TMA), Q-beta amplification, and the like.

In some embodiments, the primers and probes are used in polymerase chain reaction (PCR)-based techniques, such as RT-PCR, to detect ToBRFV infection in samples. PCR is a technique for amplifying a desired target nucleic acid sequence contained in a nucleic acid molecule or mixture of molecules. In PCR, a pair of primers is employed in excess to hybridize to the complementary strands of the target nucleic acid. The primers are each extended by a polymerase using the target nucleic acid as a template. The extension products become target sequences themselves after dissociation from the original target strand. New primers are then hybridized and extended by a polymerase, and the cycle is repeated to geometrically increase the number of target sequence molecules. The PCR method for amplifying target nucleic acid sequences in a sample is well known in the art and has been described in, e.g., Innis et al. (eds.) PCR Protocols (Academic Press, N Y 1990); Taylor (1991) Polymerase chain reaction: basic principles and automation, in PCR: A Practical Approach, McPherson et al. (eds.) IRL Press, Oxford; Saiki et al. (1986) Nature 324:163; as well as in U.S. Pat. Nos. 4,683,195, 4,683,202 and 4,889,818, all incorporated herein by reference in their entireties.

In particular, PCR uses relatively short oligonucleotide primers which flank the target nucleotide sequence to be amplified, oriented such that their 3′ ends face each other, each primer extending toward the other. The polynucleotide sample is extracted and denatured, preferably by heat, and hybridized with first and second primers that are present in molar excess. Polymerization is catalyzed in the presence of the four deoxyribonucleotide triphosphates (dNTPs—dATP, dGTP, dCTP and dTTP) using a primer- and template-dependent polynucleotide polymerizing agent, such as any enzyme capable of producing primer extension products, for example, E. coli DNA polymerase I, Klenow fragment of DNA polymerase I, T4 DNA polymerase, thermostable DNA polymerases isolated from Thermus aquaticus (Taq), available from a variety of sources (for example, Perkin Elmer), Thermus thermophilus polymerase (United States Biochemicals), Bacillus stearothermophilus polymerase (Bio-Rad), Thermococcus litoralis polymerase (“Vent” polymerase, New England Biolabs), Pyrococcus species GB-D polymerase (“Deep Vent” polymerase, New England Biolabs), Pyrococcus woesei polymerase (Pwo polymerase, Sigma-Aldrich) or Pyrococcus furiosus polymerase (Pfu polymerase from Promega Corporation). This results in two “long products” which contain the respective primers at their 5′ ends covalently linked to the newly synthesized complements of the original strands. The reaction mixture is then returned to polymerizing conditions, e.g., by lowering the temperature, inactivating a denaturing agent, or adding more polymerase, and a second cycle is initiated. The second cycle provides the two original strands, the two long products from the first cycle, two new long products replicated from the original strands, and two “short products” replicated from the long products. The short products have the sequence of the target sequence with a primer at each end. On each additional cycle, an additional two long products are produced, and a number of short products equal to the number of long and short products remaining at the end of the previous cycle. Thus, the number of short products containing the target sequence grows exponentially with each cycle. Preferably, PCR is carried out with a commercially available thermal cycler, e.g., Perkin Elmer.

RNAs may be amplified by reverse transcribing the RNA into cDNA, and then performing PCR (RT-PCR), as described above. Alternatively, a single enzyme may be used for both steps as described in U.S. Pat. No. 5,322,770, incorporated herein by reference in its entirety. RNA may also be reverse transcribed into cDNA, followed by asymmetric gap ligase chain reaction (RT-AGLCR) as described by Marshall et al. (1994) PCR Meth. App. 4:80-84.

PCR primers should be of sufficient length to provide for hybridization to complementary template DNA under annealing conditions. The primers will generally be at least 6 bp in length, including but not limited to e.g., at least 10 bp in length, at least 15 bp in length, at least 16 bp in length, at least 17 bp in length, at least 18 bp in length, at least 19 bp in length, at least 20 bp in length, at least 21 bp in length, at least 22 bp in length, at least 23 bp in length, at least 24 bp in length, at least 25 bp in length, at least 26 bp in length, at least 27 bp in length, at least 28 bp in length, at least 29 bp in length, at least 30 bp in length, and may be as long as 60 bp in length or longer, where the length of the primers will generally range from 18 to 50 bp in length, including but not limited to, e.g., from about 20 to 35 bp in length. In some instances, the template DNA may be contacted with a single primer or a set of two primers (forward and reverse primers), depending on whether primer extension, linear or exponential amplification of the template DNA is desired. Methods of PCR that may be employed in the subject methods include but are not limited to those described in U.S. Pat. Nos. 4,683,202; 4,683,195; 4,800,159; 4,965,188 and 5,512,462, the disclosures of which are herein incorporated by reference.

Alternatively, a polymerase that preferentially uses dUTP rather than dTTP can be used to perform PCR. Such polymerases include archaeal family B DNA polymerases such as Nanoarchaeum equitans B DNA polymerase, which can utilize deaminated bases such as uracil and hypoxanthine and performs PCR with higher fidelity than Thermus aquaticus (Taq) DNA polymerase (e.g., as described in Choi et al. (2008) Appl. Environ. Microbiol. 74(21): 6563-6569; herein incorporated by reference). In addition, engineered polymerases such as Q5U Hot Start High-Fidelity DNA Polymerase from New England Biolabs (Ipswich, MA) and Phusion U DNA polymerase from Thermo Fisher Scientific (Waltham, MA), which contain a mutation in the nucleotide-binding pocket that enables these polymerases to amplify templates containing uracil and inosine bases, may be used to perform PCR with dUTP. The use of polymerases that utilize UTP is useful for preventing carryover contamination in different PCR runs. The uracil-containing amplicon products of such polymerases can be digested by a uracil-DNA glycosylase to remove residual products from previous PCR amplifications and suppress template contamination between runs.

In addition, one or more PCR additives or enhancing agents may be included to improve the yield of the amplification reaction, for example, by reducing secondary structure in a nucleic acid or mispriming events. Such additives or enhancing agents include, but are not limited to, dimethyl sulfoxide (DMSO), N,N,N-trimethylglycine (betaine), formamide, glycerol, nonionic detergents (e.g., Triton X-100, Tween 20, and Nonidet P-40 (NP-40)), 7-deaza-2′-deoxyguanosine, bovine serum albumin, T4 gene 32 protein, polyethylene glycol, 1,2-propanediol, and tetramethylammonium chloride.

A PCR reaction will generally be carried out by cycling the reaction mixture between appropriate temperatures for annealing, elongation/extension, and denaturation for specific times. Such temperature and times will vary and will depend on the particular components of the reaction including, e.g., the polymerase and the primers as well as the expected length of the resulting PCR product. In some instances, e.g., where nested or two-step PCR are employed the cycling-reaction may be carried out in stages, e.g., cycling according to a first stage having a particular cycling program or using particular temperature(s) and subsequently cycling according to a second stage having a particular cycling program or using particular temperature(s).

Multistep PCR processes may or may not include that addition of one or more reagents following the initiation of amplification. For example, in some instances, amplification may be initiated by elongation with the use of a polymerase and, following an initial phase of the reaction, additional reagent(s) (e.g., one or more additional primers, additional enzymes, etc.) may be added to the reaction to facilitate a second phase of the reaction. In some instances, amplification may be initiated with a first primer or a first set of primers and, following an initial phase of the reaction, additional reagent(s) (e.g., one or more additional primers, additional enzymes, etc.) may be added to the reaction to facilitate a second phase of the reaction. In certain embodiments, the initial phase of amplification may be referred to as “preamplification”.

In particular, the subject methods are applicable to digital PCR techniques. For digital PCR, a sample containing nucleic acids is separated into a large number of partitions before performing PCR. Partitioning can be achieved in a variety of ways known in the art, for example, by use of micro well plates, capillaries, emulsions, arrays of miniaturized chambers or nucleic acid binding surfaces. Separation of the sample may involve distributing any suitable portion including up to the entire sample among the partitions. Each partition includes a fluid volume that is isolated from the fluid volumes of other partitions. The partitions may be isolated from one another by a fluid phase, such as a continuous phase of an emulsion, by a solid phase, such as at least one wall of a container, or a combination thereof. In certain embodiments, the partitions may comprise droplets disposed in a continuous phase, such that the droplets and the continuous phase collectively form an emulsion.

The partitions may be formed by any suitable procedure, in any suitable manner, and with any suitable properties. For example, the partitions may be formed with a fluid dispenser, such as a pipette, with a droplet generator, by agitation of the sample (e.g., shaking, stirring, sonication, etc.), and the like. Accordingly, the partitions may be formed serially, in parallel, or in batch. The partitions may have any suitable volume or volumes. The partitions may be of substantially uniform volume or may have different volumes. Exemplary partitions having substantially the same volume are monodisperse droplets. Exemplary volumes for the partitions include an average volume of less than about 100, 10 or 1 mL, less than about 100, 10, or 1 nL, or less than about 100, 10, or 1 pL, among others.

After separation of the sample, PCR is carried out in the partitions. The partitions, when formed, may be competent for performance of one or more reactions in the partitions. Alternatively, one or more reagents may be added to the partitions after they are formed to render them competent for reaction. The reagents may be added by any suitable mechanism, such as a fluid dispenser, fusion of droplets, or the like.

In some embodiments, nucleic acids are amplified by emulsion PCR to compartmentalize the amplification reactions of individual DNA molecules. An aqueous PCR mixture with forward and reverse primers is mixed with an oil to create the emulsion. Preferably, each droplet of water in the oil emulsion contains one bead and one molecule of template DNA (e.g., a single assembled nucleic acid of the sequencing library), such that individual molecules are amplified in separate emulsion droplets. After amplification, the emulsion is broken, e.g., using isopropanol and detergent with vortexing. In some embodiments, the gene fragment library and the sequencing library are bound to magnetic beads or superparamagnetic beads prior to amplification, wherein amplification and breaking of the emulsion is followed by magnetic separation of the beads. For a description of emulsion PCR, see, e.g., Kanagal-Shamanna et al. (2016) Methods Mol Biol. 1392:33-42, Zhu et al. (2012) Anal Bioanal Chem. 403(8):2127-43, Zhang et al. (2020) Lab Chip 20(13):2328-2333, Siu et al. (2021) Talanta 221:121593, Zheng et al. (2011) Nat. Protoc. 6(9):1367-1376, and Kojima et al. (2015) Methods Mol. Biol. 2015; 1347:87-100; herein incorporated by reference.

After PCR amplification, nucleic acids can be quantified by counting the partitions that contain PCR amplicons. Partitioning of the sample allows quantification of the number of different molecules by assuming that the population of molecules follows a Poisson distribution. For a description of digital PCR methods, see, e.g., Hindson et al. (2011) Anal. Chem. 83(22):8604-8610; Pohl and Shih (2004) Expert Rev. Mol. Diagn. 4(1):41-47; Pekin et al. (2011) Lab Chip 11 (13): 2156-2166; Pinheiro et al. (2012) Anal. Chem. 84 (2): 1003-1011; Day et al. (2013) Methods 59(1):101-107; herein incorporated by reference in their entireties.

In some instances, amplification may be carried out under isothermal conditions, e.g., by means of isothermal amplification. Methods of isothermal amplification generally make use of enzymatic means of separating DNA strands to facilitate amplification at constant temperature, such as, e.g., strand-displacing polymerase or a helicase, thus negating the need for thermocycling to denature DNA. Any convenient and appropriate means of isothermal amplification may be employed in the subject methods including but are not limited to: recombinase polymerase amplification (RPA), loop-mediated isothermal amplification (LAMP), strand displacement amplification (SDA), helicase-dependent amplification (HDA), nicking enzyme amplification reaction (NEAR), ligase chain reaction (LGR), nucleic acid sequence based amplification (NASBA), transcription-mediated amplification (TMA), Q-beta amplification, and the like.

RPA combines isothermal recombinase-mediated primer targeting with strand-displacement DNA synthesis (Piepenburg et al. (2006) PLoS Biology. 4 (7): e204; herein incorporated by reference). The technique uses two primers together with a recombinase, a single-stranded DNA-binding protein, and a strand-displacing polymerase for amplification. Unlike PCR, heat is not required for melting of the DNA strands. Instead, a recombinase-primer complex is used for localized strand exchange to place oligonucleotide primers at homologous sequences of the DNA template. The single-stranded DNA-binding protein binds to the displaced template strand to prevent the primers from being ejected by branch migration. Dissociation of the recombinase leaves the 3′-end of the primer accessible to the strand displacing DNA polymerase (e.g., the large fragment of Bacillus subtilis Pol I), which catalyzes primer extension. Cyclic repetition of this process results in exponential amplification.

LAMP generally utilizes a plurality of primers, e.g., 4-6 primers, which may recognize a plurality of distinct regions, e.g., 6-8 distinct regions, of target DNA. Synthesis is generally initiated by a strand-displacing DNA polymerase with two of the primers forming loop structures to facilitate subsequent rounds of amplification. LAMP is rapid and sensitive. In addition, the magnesium pyrophosphate produced during the LAMP amplification reaction may, in some instances, be visualized without the use of specialized equipment, e.g., by eye.

SDA generally involves the use of a strand-displacing DNA polymerase (e.g., Bst DNA polymerase, Large (Klenow) Fragment polymerase, Klenow Fragment (3′-5′ exo-), and the like) to initiate at nicks created by a strand-limited restriction endonuclease or nicking enzyme at a site contained in a primer. In SDA, the nicking site is generally regenerated with each polymerase displacement step, resulting in exponential amplification.

HDA generally employs: a helicase which unwinds double-stranded DNA unwinding to separate strands; primers, e.g., two primers, that may anneal to the unwound DNA; and a strand-displacing DNA polymerase for extension.

NEAR generally involves a strand-displacing DNA polymerase that initiates elongation at nicks, e.g., created by a nicking enzyme. NEAR is rapid and sensitive, quickly producing many short nucleic acids from a target sequence.

Nucleic acid sequence-based amplification (NASBA) is an isothermal RNA-specific amplification method that does not require thermal cycling instrumentation. RNA is initially reverse transcribed such that the single-stranded RNA target is copied into a double-stranded DNA molecule that serves as a template for RNA transcription. Detection of the amplified RNA is typically accomplished either by electrochemiluminescence or in real-time, for example, with fluorescently labeled molecular beacon probes. See, e.g., Lau et al. (2006) Dev. Biol. (Basel) 126:7-15; and Deiman et al. (2002) Mol. Biotechnol. 20(2):163-179.

The Ligase Chain Reaction (LCR) is an alternate method for nucleic acid amplification. In LCR, probe pairs are used which include two primary (first and second) and two secondary (third and fourth) probes, all of which are employed in molar excess to the target. The first probe hybridizes to a first segment of the target strand, and the second probe hybridizes to a second segment of the target strand, the first and second segments being contiguous so that the primary probes abut one another in 5′ phosphate-3′ hydroxyl relationship, and so that a ligase can covalently fuse or ligate the two probes into a fused product. In addition, a third (secondary) probe can hybridize to a portion of the first probe and a fourth (secondary) probe can hybridize to a portion of the second probe in a similar abutting fashion. If the target is initially double stranded, the secondary probes also will hybridize to the target complement in the first instance. Once the ligated strand of primary probes is separated from the target strand, it will hybridize with the third and fourth probes which can be ligated to form a complementary, secondary ligated product. It is important to realize that the ligated products are functionally equivalent to either the target or its complement. By repeated cycles of hybridization and ligation, amplification of the target sequence is achieved. This technique is described more completely in EPA 320,308 to K. Backman published Jun. 16, 1989 and EPA 439,182 to K. Backman et al., published Jul. 31, 1991, both of which are incorporated herein by reference.

Other known methods for amplification of nucleic acids include, but are not limited to, self-sustained sequence replication (3SR) described by Guatelli et al., Proc. Natl. Acad. Sci. USA (1990) 87:1874-1878 and J. Compton, Nature (1991) 350:91-92 (1991); Q-beta amplification; strand displacement amplification (as described in Walker et al., Clin. Chem. (1996) 42:9-13 and EPA 684,315; target mediated amplification, as described in International Publication No. WO 93/22461, and the TaqMan™ assay.

In some instances, entire amplification methods may be combined or aspects of various amplification methods may be recombined to generate a hybrid amplification method. For example, in some instances, aspects of PCR may be used, e.g., to generate the initial template or amplicon or first round or rounds of amplification, and an isothermal amplification method may be subsequently employed for further amplification. In some instances, an isothermal amplification method or aspects of an isothermal amplification method may be employed, followed by PCR for further amplification of the product of the isothermal amplification reaction. In some instances, a sample may be preamplified using a first method of amplification and may be further processed, including e.g., further amplified or analyzed, using a second method of amplification. As a non-limiting example, a sample may be preamplified by PCR and further analyzed by qPCR. In some instances, the method further comprises monitoring the amplification of a target DNA molecule such as is performed in, e.g., real-time PCR, also referred to herein as quantitative PCR (qPCR).

The fluorogenic 5′ nuclease assay, known as the TaqMan™ assay (Perkin-Elmer), is a powerful and versatile PCR-based detection system for nucleic acid targets. Primers and probes derived from conserved and/or non-conserved regions of the ToBRFV genome in question can be used in TaqMan™ analyses to detect the presence of human feces in a sample. Analysis is performed in conjunction with thermal cycling by monitoring the generation of fluorescence signals. The assay system dispenses with the need for gel electrophoretic analysis, and is capable of generating quantitative data allowing the determination of target copy numbers. For example, standard curves can be produced using serial dilutions of previously quantified ToBRFV viral suspensions. A standard graph can be produced with copy numbers of each of the panel members against which sample unknowns can be compared.

The fluorogenic 5′ nuclease assay is conveniently performed using, for example, AmpliTaq Gold™ DNA polymerase, which has endogenous 5′ nuclease activity, to digest an internal oligonucleotide probe labeled with both a fluorescent reporter dye and a quencher (see, Holland et al., Proc. Natl. Acad. Sci. USA (1991) 88:7276-7280; and Lee et al., Nucl. Acids Res. (1993) 21:3761-3766). Assay results are detected by measuring changes in fluorescence that occur during the amplification cycle as the fluorescent probe is digested, uncoupling the dye and quencher labels and causing an increase in the fluorescent signal that is proportional to the amplification of target nucleic acid.

The amplification products can be detected in solution or using solid supports. In this method, the TaqMan™ probe is designed to hybridize to a target sequence within the desired PCR product. The 5′ end of the TaqMan™ probe contains a fluorescent reporter dye. The 3′ end of the probe is blocked to prevent probe extension and contains a dye that will quench the fluorescence of the 5′ fluorophore. During subsequent amplification, the 5′ fluorescent label is cleaved off if a polymerase with 5′ exonuclease activity is present in the reaction. Excision of the 5′ fluorophore results in an increase in fluorescence that can be detected.

For a detailed description of the TaqMan™ assay, reagents and conditions for use therein, see, e.g., Holland et al., Proc. Natl. Acad. Sci, U.S.A. (1991) 88:7276-7280; U.S. Pat. Nos. 5,538,848, 5,723,591, and 5,876,930, all incorporated herein by reference in their entireties.

A class of quenchers, known as “Black Hole Quenchers” such as BHQ®-0, BHQ®-1, BHQ®-2, BHQ®-3, BHQplus®, and BHQnova® can be used in the nucleic acid assays described above. These quenchers reduce background and improve signal to noise in PCR assays. These quenchers are described in, e.g., Johansson et al., J. Chem. Soc. (2002) 124:6950-6956 and are commercially available from LGC Biosearch Technologies (Hoddesdon, United Kingdom). Other quenchers that can be used in the nucleic acid assays include, but are not limited to, BlackBerry™ Quenchers such as BBQ-650; Eclipse® Quencher, ATTO quenchers such as ATTO 540Q, ATTO 575Q, ATTO 580Q, and ATTO 612Q, and MB2, TAMRA, and dabcyl.

While the length of the primers and probes can vary, the probe sequences are selected such that they have a higher melt temperature than the primer sequences. Preferably, the probe sequences have an estimated melt temperature that is about 10° C. higher than the melt temperature for the amplification primer sequences. Hence, the primer sequences are generally shorter than the probe sequences. Typically, the primer sequences are in the range of between 10-75 nucleotides long, more typically in the range of 20-45. The typical probe is in the range of between 10-50 nucleotides long, more typically 15-40 nucleotides in length. Representative primers and probes useful in nucleic acid amplification assays are described above.

The ToBRFV sequences described herein may also be used as a basis for transcription-mediated amplification (TMA) assays. TMA is an isothermal, autocatalytic nucleic acid target amplification system that can provide more than a billion RNA copies of a target sequence, and thus provides a method of identifying target nucleic acid sequences present in very small amounts in a sample. For a detailed description of TMA assay methods, see, e.g., Hill (2001) Expert Rev. Mol. Diagn. 1:445-55; WO 89/1050; WO 88/10315; EPO Publication No. 408,295; EPO Application No. 8811394-8.9; WO91/02818; U.S. Pat. Nos. 5,399,491, 6,686,156, and 5,556,771, all incorporated herein by reference in their entireties.

Suitable DNA polymerases include reverse transcriptases, such as avian myeloblastosis virus (AMV) reverse transcriptase (available from, e.g., Seikagaku America, Inc.) and Moloney murine leukemia virus (MMLV) reverse transcriptase (available from, e.g., Bethesda Research Laboratories).

Promoters or promoter sequences suitable for incorporation in the primers are nucleic acid sequences (either naturally occurring, produced synthetically or a product of a restriction digest) that are specifically recognized by an RNA polymerase that recognizes and binds to that sequence and initiates the process of transcription whereby RNA transcripts are produced. The sequence may optionally include nucleotide bases extending beyond the actual recognition site for the RNA polymerase which may impart added stability or susceptibility to degradation processes or increased transcription efficiency. Examples of useful promoters include those which are recognized by certain bacteriophage polymerases such as those from bacteriophage T3, T7 or SP6, or a promoter from E. coli. These RNA polymerases are readily available from commercial sources, such as New England Biolabs and Epicentre.

Some of the reverse transcriptases suitable for use in the methods herein have an RNAse H activity, such as AMV reverse transcriptase. It may, however, be preferable to add exogenous RNAse H, such as E. coli RNAse H, even when AMV reverse transcriptase is used. RNAse H is readily available from, e.g., Bethesda Research Laboratories.

The RNA transcripts produced by these methods may serve as templates to produce additional copies of the target sequence through the above-described mechanisms. The system is autocatalytic and amplification occurs autocatalytically without the need for repeatedly modifying or changing reaction conditions such as temperature, pH, ionic strength or the like.

Detection may be done using a wide variety of methods, including direct sequencing, hybridization with sequence-specific oligomers, gel electrophoresis and mass spectrometry. These methods can use heterogeneous or homogeneous formats, isotopic or nonisotopic labels, as well as no labels at all.

One method of detection is the use of target sequence-specific oligonucleotide probes described above. The probes may be used in hybridization protection assays (HPA). In this embodiment, the probes are conveniently labeled with acridinium ester (AE), a highly chemiluminescent molecule. See, e.g., Nelson et al. (1995) “Detection of Acridinium Esters by Chemiluminescence” in Nonisotopic Probing, Blotting and Sequencing, Kricka L. J. (ed) Academic Press, San Diego, Calif.; Nelson et al. (1994) “Application of the Hybridization Protection Assay (HPA) to PCR” in The Polymerase Chain Reaction, Mullis et al. (eds.) Birkhauser, Boston, Mass.; Weeks et al., Clin. Chem. (1983) 29:1474-1479; Berry et al., Clin. Chem. (1988) 34:2087-2090. One AE molecule is directly attached to the probe using a non-nucleotide-based linker arm chemistry that allows placement of the label at any location within the probe. See, e.g., U.S. Pat. Nos. 5,585,481 and 5,185,439. Chemiluminescence is triggered by reaction with alkaline hydrogen peroxide which yields an excited N-methyl acridone that subsequently collapses to ground state with the emission of a photon.

When the AE molecule is covalently attached to a nucleic acid probe, hydrolysis is rapid under mildly alkaline conditions. When the AE-labeled probe is exactly complementary to the target nucleic acid, the rate of AE hydrolysis is greatly reduced. Thus, hybridized and unhybridized AE-labeled probe can be detected directly in solution, without the need for physical separation.

HPA generally consists of the following steps: (a) the AE-labeled probe is hybridized with the target nucleic acid in solution for about 15 to about 30 minutes. A mild alkaline solution is then added and AE coupled to the unhybridized probe is hydrolyzed. This reaction takes approximately 5 to 10 minutes. The remaining hybrid-associated AE is detected as a measure of the amount of target present. This step takes approximately 2 to 5 seconds. Preferably, the differential hydrolysis step is conducted at the same temperature as the hybridization step, typically at 50 to 70° C. Alternatively, a second differential hydrolysis step may be conducted at room temperature. This allows elevated pHs to be used, for example in the range of 10-11, which yields larger differences in the rate of hydrolysis between hybridized and unhybridized AE-labeled probe. HPA is described in detail in, e.g., U.S. Pat. Nos. 6,004,745; 5,948,899; and 5,283,174, the disclosures of which are incorporated by reference herein in their entireties.

In one example of a typical TMA assay, an isolated nucleic acid sample, suspected of containing a ToBRFV target sequence, is mixed with a buffer concentrate containing the buffer, salts, magnesium, nucleotide triphosphates, primers, dithiothreitol, and spermidine. The reaction is optionally incubated at about 100° C. for approximately two minutes to denature any secondary structure. After cooling to room temperature, reverse transcriptase, RNA polymerase, and RNAse H are added and the mixture is incubated for two to four hours at 37° C. The reaction can then be assayed by denaturing the product, adding a probe solution, incubating 20 minutes at 60° C., adding a solution to selectively hydrolyze the unhybridized probe, incubating the reaction six minutes at 60° C., and measuring the remaining chemiluminescence in a luminometer.

The methods of detection of the invention utilize a sample suspected of containing human fecal contamination comprising ToBRFV nucleic acids. A sample may be pre-treated in any number of ways prior to assaying for ToBRFV nucleic acids. For instance, in certain embodiments, the sample may be treated to disrupt (or lyse) any viral particles (virions), for example by treating the samples with one or more detergents and/or denaturing agents (e.g., guanidinium agents). Nucleic acids may also be extracted from samples, for example, after detergent treatment and/or denaturing as described above. Total nucleic acid extraction may be performed using known techniques, for example by non-specific binding to a solid phase (e.g., silica). See, e.g., U.S. Pat. Nos. 5,234,809, 6,849,431; 6,838,243; 6,815,541; and 6,720,166.

In certain embodiments, the target nucleic acids are separated from non-homologous nucleic acids using capture oligonucleotides immobilized on a solid support. Such capture oligonucleotides contain nucleic acid sequences that are complementary to a nucleic acid sequence present in the target ToBRFV nucleic acid analyte such that the capture oligonucleotide can “capture” the target nucleic acid. Capture oligonucleotides can be used alone or in combination to capture ToBRFV nucleic acids. For example, multiple capture oligonucleotides can be used in combination, e.g., 2, 3, 4, 5, 6, etc. different capture oligonucleotides can be attached to a solid support to capture target ToBRFV nucleic acids. In certain embodiments, one or more capture oligonucleotides can be used to bind ToBRFV target nucleic acids either prior to or after amplification by primer oligonucleotides and/or detection by probe oligonucleotides.

In one embodiment, the sample potentially carrying target nucleic acids is contacted with a solid support in association with capture oligonucleotides. The capture oligonucleotides, which may be used separately or in combination, may be associated with the solid support, for example, by covalent binding of the capture moiety to the solid support, by affinity association, hydrogen binding, or nonspecific association.

The capture oligonucleotides can include from about 5 to about 500 nucleotides of a conserved region from a ToBRFV, preferably about 10 to about 100 nucleotides, or more preferably about 10 to about 60 nucleotides of the conserved region, or any integer within these ranges, such as a sequence including 18, 19, 20, 21, 22, 23, 24, 25, 26 . . . 35 . . . 40, etc. nucleotides from the conserved region of interest. In certain embodiments, the capture oligonucleotide comprises a sequence selected from the group consisting of SEQ ID NOS:5-21 or a complement thereof. The capture oligonucleotide may also be phosphorylated at the 3′ end in order to prevent extension of the capture oligonucleotide.

The capture oligonucleotide may be attached to the solid support in a variety of manners. For example, the oligonucleotide may be attached to the solid support by attachment of the 3′ or 5′ terminal nucleotide of the probe to the solid support. More preferably, the capture oligonucleotide is attached to the solid support by a linker which serves to distance the probe from the solid support. The linker is usually at least 10-50 atoms in length, more preferably at least 15-30 atoms in length. The required length of the linker will depend on the particular solid support used. For example, a six atom linker is generally sufficient when high cross-linked polystyrene is used as the solid support.

A wide variety of linkers are known in the art which may be used to attach the oligonucleotide probe to the solid support. The linker may be formed of any compound which does not significantly interfere with the hybridization of the target sequence to the probe attached to the solid support. The linker may be formed of a homopolymeric oligonucleotide which can be readily added on to the linker by automated synthesis. The homopolymeric sequence can be either 5′ or 3′ to the virus-specific sequence. In one aspect of the invention, the capture oligonucleotides include a homopolymer chain, such as, for example poly A, poly T, poly G, poly C, poly U, poly dA, poly dT, poly dG, poly dC, or poly dU in order to facilitate attachment to a solid support. The homopolymer chain can be from about 10 to about 40 nucleotides in length, or preferably about 12 to about 25 nucleotides in length, or any integer within these ranges, such as for example, 10 . . . 12 . . . 16, 17, 18, 19, 20, 21, 22, 23, or 24 nucleotides. The homopolymer, if present, can be added to the 3′ or 5′ terminus of the capture oligonucleotides by enzymatic or chemical methods. This addition can be made by stepwise addition of nucleotides or by ligation of a preformed homopolymer. Capture oligonucleotides comprising such a homopolymer chain can be bound to a solid support comprising a complementary homopolymer. Alternatively, biotinylated capture oligonucleotides can be bound to avidin- or streptavidin-coated beads. See, e.g., Chollet et al., supra.

Alternatively, polymers such as functionalized polyethylene glycol can be used as the linker Such polymers do not significantly interfere with the hybridization of probe to the target oligonucleotide. Examples of linkages include polyethylene glycol, carbamate and amide linkages. The linkages between the solid support, the linker and the probe are preferably not cleaved during removal of base protecting groups under basic conditions at high temperature.

The solid support may take many forms including, for example, nitrocellulose reduced to particulate form and retrievable upon passing the sample medium containing the support through a sieve; nitrocellulose or the materials impregnated with magnetic particles or the like, allowing the nitrocellulose to migrate within the sample medium upon the application of a magnetic field; beads or particles which may be filtered or exhibit electromagnetic properties; and polystyrene beads which partition to the surface of an aqueous medium. Examples of types of solid supports for immobilization of the oligonucleotide probe include controlled pore glass, glass plates, polystyrene, avidin-coated polystyrene beads, cellulose, nylon, acrylamide gel and activated dextran.

In one embodiment, the solid support comprises magnetic beads. The magnetic beads may contain primary amine functional groups, which facilitate covalent binding or association of the capture oligonucleotides to the magnetic support particles. Alternatively, the magnetic beads have immobilized thereon homopolymers, such as poly T or poly A sequences. The homopolymers on the solid support will generally be complementary to any homopolymer on the capture oligonucleotide to allow attachment of the capture oligonucleotide to the solid support by hybridization. The use of a solid support with magnetic beads allows for a one-pot method of isolation, amplification and detection as the solid support can be separated from the sample by magnetic means.

The magnetic beads or particles can be produced using standard techniques or obtained from commercial sources. In general, the particles or beads may be comprised of magnetic particles, although they can also include other magnetic metal or metal oxides, whether in impure, alloy, or composite form, as long as they have a reactive surface and exhibit an ability to react to a magnetic field. Other materials that may be used individually or in combination with iron include, but are not limited to, cobalt, nickel, and silicon. A magnetic bead suitable for use with the present invention includes magnetic beads containing poly dT groups marketed under the trade name Sera-Mag magnetic oligonucleotide beads by Seradyn, Indianapolis, Ind.

Next, the association of the capture oligonucleotides with the solid support is initiated by contacting the solid support with the medium containing the capture oligonucleotides. In the preferred embodiment, the magnetic beads containing poly dT groups are hybridized with the capture oligonucleotides that comprise poly dA contiguous with the capture sequence (i.e., the sequence substantially complementary to a ToBRFV nucleic acid sequence) selected from the conserved single stranded region of the ToBRFV genome. The poly dA on the capture oligonucleotide and the poly dT on the solid support hybridize thereby immobilizing or associating the capture oligonucleotides with the solid support.

In certain embodiments, the capture oligonucleotides are combined with a sample under conditions suitable for hybridization with target ToBRFV nucleic acids prior to immobilization of the capture oligonucleotides on a solid support. The capture oligonucleotide-target nucleic acid complexes formed are then bound to the solid support. In other embodiments, a solid support with associated capture oligonucleotides is brought into contact with a sample under hybridizing conditions. The immobilized capture oligonucleotides hybridize to the target nucleic acids present in the sample. Typically, hybridization of capture oligonucleotides to the targets can be accomplished in approximately 15 minutes, but may take as long as 3 to 48 hours.

The solid support is then separated from the sample, for example, by filtering, centrifugation, passing through a column, or by magnetic means. The solid support maybe washed to remove unbound contaminants and transferred to a suitable container (e.g., a microtiter plate). As will be appreciated by one of skill in the art, the method of separation will depend on the type of solid support selected. Since the targets are hybridized to the capture oligonucleotides immobilized on the solid support, the target strands are thereby separated from the impurities in the sample. In some cases, extraneous nucleic acids, proteins, carbohydrates, lipids, cellular debris, and other impurities may still be bound to the support, although at much lower concentrations than initially found in the sample. Those skilled in the art will recognize that some undesirable materials can be removed by washing the support with a washing medium. The separation of the solid support from the sample preferably removes at least about 70%, more preferably about 90% and, most preferably, at least about 95% or more of the non-target nucleic acids present in the sample.

As is readily apparent, design of the assays described herein is subject to a great deal of variation, and many formats are known in the art. The above descriptions are merely provided as guidance and one of skill in the art can readily modify the described protocols, using techniques well known in the art.

### Kits

The above-described assay reagents, including the primers and probes, and optionally capture oligonucleotides, a solid support with bound probes, and/or reagents for performing nucleic acid amplification, such as by RT-PCR or isothermal amplification (e.g., NASBA), or other amplification method, can be provided in kits, with suitable instructions and other necessary reagents, in order to conduct the assays as described above. The kit will normally contain in separate containers the primers and probes, control formulations (positive and/or negative), and other reagents that the assay format requires. The kit can also contain, depending on the particular assay used, other packaged reagents and materials (i.e., wash buffers, and the like). The reagents may be provided independently in liquid or solid form or provided in mixtures. Standard assays, such as those described above, can be conducted using these kits.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, Blu-ray, flash drive, and the like, on which the information has been recorded. Yet another form of these instructions that may be present is a website address which may be used via the internet to access the information at a removed site.

In certain embodiments, the kit comprises instructions for detecting human fecal contamination in a sample based on the presence of ToBRFV RNA and at least one set of primers comprising a forward primer and a reverse primer capable of selectively amplifying at least a portion of a movement protein (Mo) encoding gene or a portion of an RNA dependent RNA polymerase (RdRP) encoding gene, or a combination. In certain embodiments, the kit comprises a set of primers comprising: (a) a forward primer comprising or consisting of the sequence of SEQ ID NO:1 and a reverse primer comprising or consisting of the sequence of SEQ ID NO:2; (b) a forward primer comprising or consisting of the sequence of SEQ ID NO:4 and a reverse primer comprising or consisting of the sequence of SEQ ID NO:5; (c) a forward primer and a reverse primer each comprising at least 10 contiguous nucleotides from the corresponding nucleotide sequences of the forward primer and reverse primer of a primer set selected from the group consisting of (a) and (b); (d) a forward primer and a reverse primer comprising at least one nucleotide sequence that differs from the corresponding nucleotide sequence of the forward primer or reverse primer of a primer set selected from the group consisting of (a)-(c) in that the primer has up to three nucleotide changes compared to the corresponding sequence, wherein the primer is capable of hybridizing to and amplifying ToBRFV nucleic acids in the nucleic acid amplification assay; (e) a forward primer and a reverse primer comprising nucleotide sequences that are complements of the corresponding nucleotide sequences of the forward primer and reverse primer of a primer set selected from the group consisting of (a)-(d); or (f) a combination of a primer set selected from the group comprising (a)-(e).

In certain embodiments, the kit comprises a set of primers comprising or consisting of the sequence of SEQ ID NO:1, a primer comprising or consisting of the sequence of SEQ ID NO:2, a primer comprising or consisting of the sequence of SEQ ID NO:4, and a primer comprising or consisting of the sequence of SEQ ID NO:5.

In certain embodiments, the kit comprises at least one oligonucleotide probe selected from the group consisting of: (a) a probe comprising or consisting of the sequence of SEQ ID NO:3, (b) a probe comprising or consisting of a sequence selected from the group consisting of SEQ ID NO:6 and SEQ ID NO:7, (c) a probe that differs from the corresponding nucleotide sequence of a probe selected from the group consisting of (a) and (b) in that the probe has up to three nucleotide changes compared to the corresponding sequence, wherein the probe is capable of hybridizing to and detecting the ToBRFV RNA or amplicon thereof, and (d) a combination of probes selected from the group comprising (a)-(c). In some embodiments, said at least one oligonucleotide probe comprises a probe comprising or consisting of the sequence of SEQ ID NO:3 and a probe comprising or consisting of a sequence selected from the group consisting of SEQ ID NO:6 and SEQ ID NO:7.

### Utility

The assays disclosed herein, using ToBRFV as an MST marker of human feces, can be used to detect human fecal contamination in any type of sample, including, without limitation, samples of water, dirt, sludge, air, or on surfaces of fomites. The subject methods can further be used to distinguish fecal contamination from humans and non-human animals. In some embodiments, ToBRFV is used as an MST marker of human feces in combination with one or more MST markers for one or more non-human animals (e.g., wild animals, agricultural animals, pets, birds, etc.) to identify the source of fecal contamination. For example, ToBRFV can be used as an MST marker of human feces in combination with one or more MST markers for one or more animals such as, but not limited to, cows, dogs, cats, rats, mice, guinea-pigs, goats, horses, sheep, pigs, bears, deer, elk, bison, buffalos, antelopes, chickens, turkeys, ducks, pigeons, gulls, or parrots.

The subject methods are useful for detecting fecal contamination in water, which may be unsafe for drinking, bathing, or recreational use due to elevated fecal contamination from, for example, sewer overflows, leaking septic systems, fecal contamination from pets, local wildlife, or nearby agricultural practices. The methods can also be used to monitor food preparation surfaces and sanitation practices at restaurants, groceries, bakeries, and other businesses that supply or sell food; medical equipment at hospitals such as, but not limited to, stethoscopes, intravenous (IV) drip tubes, catheters, and life support equipment; and fomites (e.g., frequently touched items) that require sanitation in the hospitality industry (e.g., at hotels, motels, or other lodging) such as, but not limited to, bedding, furniture, bathroom faucet handles, toilet flush levers, door knobs, light switches, handrails, elevator buttons, televisions, remote controls, common-use phones, glassware, serveware, utensils, and plates.

The assays described herein are also useful for wastewater surveillance of pathogens that are excreted in human feces to provide indications of changes in community levels of an infectious disease. Such pathogens can be quantitated in wastewater based on the presence of biomarkers such as microbial DNA or RNA that are shed into the wastewater. The disclosed methods can be used to determine the amount of a pathogen per the amount of human feces in wastewater. For example, the measured concentrations of the pathogen can be divided by the concentration of ToBRFV RNA to control for sample-to-sample variation in the amount of human sewage in wastewater and nucleic acid extraction efficiency to provide a normalized value that correlates with disease incidence rates. This method can be applied to wastewater surveillance of any type of pathogen that is excreted in human feces such as viruses, bacteria, fungi, and parasites. For example, this method can be applied to wastewater surveillance of a virus such as, but not limited to, an enterovirus (e.g., poliovirus, coxsackie A virus, coxsackie B virus, or echovirus), a rotavirus, a parvovirus-like virus, an astrovirus, a calicivirus, an adenovirus, a norovirus, or a coronavirus (e.g., severe acute respiratory syndrome coronavirus 2 (SARS-CoV-2)). In some embodiments, this method is applied to wastewater surveillance of SARS-CoV-2, hepatitis A virus, hepatitis B virus, hepatitis E virus, herpesvirus, influenza A, influenza B, respiratory syncytial virus (RSV), monkeypox virus (MPXV), dengue virus, yellow fever virus, zika virus, or varicella-zoster virus. In some embodiments, this method is applied to wastewater surveillance of bacteria such as, but not limited to, Escherichia coli, Salmonella, Shigella, Vibrio, Campylobacter, Yersinia, or Clostridium. In some embodiments, this method is applied to wastewater surveillance of antibiotic-resistant bacteria. In some embodiments, this method is applied to wastewater surveillance of fungi such as, but not limited to, Candida auris, Blastomyces dermatitidis, Blastomyces gilchristii, or Cryptococcus neoformans. In some embodiments, this method is applied to wastewater surveillance of parasites such as, but not limited to, Entamoeba histolytica, Cryptosporidium parvum, Cyclospora cayetanensis, Giardia duodenalis, or Plasmodium falciparum.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-76 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.
1. A method for selectively detecting tomato brown rugose fruit virus (ToBRFV) in a sample suspected of having human fecal contamination, the method comprising:
isolating nucleic acids from the sample suspected of having human fecal contamination, wherein if ToBRFV RNA is present, said nucleic acids comprise a target sequence;
amplifying the nucleic acids using a set of primers capable of selectively amplifying at least a portion of the ToBRFV RNA, wherein the ToBRFV RNA comprise the target sequence; and
detecting the presence of the amplified nucleic acids using a detectably labeled oligonucleotide probe sufficiently complementary to and capable of hybridizing with the ToBRFV RNA or amplicon thereof, if present, as an indication of the presence or absence of the ToBRFV in the sample, wherein said primers and said probe are capable of selectively hybridizing to the target sequence from the ToBRFV, wherein said detecting the presence of the ToBRFV in the sample indicates that human feces is present in the sample.
2. The method of aspect 1, wherein the set of primers is capable of selectively amplifying at least a portion of a movement protein (Mo) encoding gene or at least a portion of an RNA dependent RNA polymerase (RdRP) encoding gene of the ToBRFV.
3. The method of aspect 2, wherein the set of primers comprises:
(a) a forward primer comprising or consisting of the sequence of SEQ ID NO:1 and a reverse primer comprising or consisting of the sequence of SEQ ID NO:2;
(b) a forward primer comprising or consisting of the sequence of SEQ ID NO:4 and a reverse primer comprising or consisting of the sequence of SEQ ID NO:5;
(c) a forward primer and a reverse primer each comprising at least 10 contiguous nucleotides from the corresponding nucleotide sequences of the forward primer and reverse primer of a primer set selected from the group consisting of (a) and (b);
(d) a forward primer and a reverse primer comprising at least one nucleotide sequence that differs from the corresponding nucleotide sequence of the forward primer or reverse primer of a primer set selected from the group consisting of (a)-(c) in that the primer has up to three nucleotide changes compared to the corresponding sequence, wherein the primer is capable of hybridizing to and amplifying the ToBRFV nucleic acids;
(e) a forward primer and a reverse primer comprising or consisting of nucleotide sequences that are complements of the corresponding nucleotide sequences of the forward primer and reverse primer of a primer set selected from the group consisting of (a)-(d); or
(f) a combination of a primer set selected from the group consisting of (a)-(e).
4. The method of aspect 3, wherein the set of primers used for detecting ToBRFV in the sample comprises a primer comprising or consisting of the sequence of SEQ ID NO:1, a primer comprising or consisting of the sequence of SEQ ID NO:2, a primer comprising or consisting of the sequence of SEQ ID NO:4, and a primer comprising or consisting of the sequence of SEQ ID NO:5.
5. The method of any one of aspects 1-4, wherein said at least one oligonucleotide probe is selected from the group consisting of:
(a) a probe comprising or consisting of the sequence of SEQ ID NO:3,
(b) a probe comprising or consisting of a sequence selected from the group consisting of SEQ ID NO:6 and SEQ ID NO:7,
(c) a probe that differs from the corresponding nucleotide sequence of a probe selected from the group consisting of (a) and (b) in that the probe has up to three nucleotide changes compared to the corresponding sequence, wherein the probe is capable of hybridizing to and detecting the ToBRFV RNA or amplicon thereof, and
(d) a combination of probes selected from the group consisting of (a)-(c).
6. The method of aspect 5, wherein said at least one oligonucleotide probe comprises a probe comprising or consisting of the sequence of SEQ ID NO:3 and a probe comprising or consisting of the sequence selected from the group consisting of SEQ ID NO:6 and SEQ ID NO:7.
7. The method of any one of aspects 1-6, said at least one probe is detectably labeled with a fluorophore.
8. The method of aspect 7, wherein each probe is detectably labeled with a different fluorophore.
9. The method of aspect 7 or 8, wherein each probe is detectably labeled with a 5′-fluorophore and a 3′-quencher.
10. The method of any one of aspects 1-9, wherein the sample is a stool sample or an environmental sample.
11. The method of aspect 10, wherein the environmental sample is a water sample, earth sample, sludge sample, air sample, surface sample, or a fomite sample.
12. The method of aspect 11, wherein the water sample is wastewater, stormwater, ocean water, lake water, river water, creek water, drinking water, recreational water, ground water, source water, stored water, seepage water, surface water, or water from a water distribution system or sewage and waste water treatment system.
13. The method of aspect 12, wherein the earth sample is a soil sample, a sand sample, a mud sample, a sediment sample, or a rock sample.
14. The method of aspect 13, wherein the surface sample is from a food preparation surface.
15. The method of aspect 14, wherein the air sample comprises aerosolized stool matter.
16. The method of any one of aspects 1-15, wherein the fomite sample is from clothes, bedding, a utensil, a cup, furniture, a vehicle, a shovel, a bowl, a bucket, a brush, a tack, a clipper, a pencil, a bath faucet handle, a toilet flush lever, a door knob, a light switch, a handrail, an elevator button, a television, a remote control, a pen, a touch screen, a common-use phone, a keyboard, a computer mouse, a coffeepot handle, a countertop, a drinking fountain, medical equipment, or any other object that is frequently touched by people.
17. The method of any one of aspects 1-16, wherein said amplifying comprises performing polymerase chain reaction (PCR) or isothermal amplification.
18. The method of aspect 17, wherein the PCR is reverse transcriptase polymerase chain reaction (RT-PCR), droplet digital polymerase chain reaction (ddPCR), or quantitative PCR (qPCR).
19. The method of aspect 18, wherein the qPCR uses a fluorogenic 5′ nuclease assay.
20. The method of any one of aspects 1-19, wherein the isothermal amplification is nucleic acid sequence-based amplification (NASBA), transcription-mediated amplification (TMA), recombinase polymerase amplification (RPA), loop-mediated isothermal amplification (LAMP), strand displacement amplification (SDA), helicase-dependent amplification (HDA), nicking enzyme amplification reaction (NEAR), ligase chain reaction (LGR), or Q-beta amplification.
21. The method of any one of aspects 1-20, further comprising quantifying the amount of the ToBRFV RNA or amplicon thereof, wherein the amount of the ToBRFV RNA or amplicon thereof is indicative of the amount of the ToBRFV in the sample.
22. The method of aspect 21, further comprising correlating the amount of the ToBRFV in the sample with an amount of human feces in the sample.
23. The method of aspect 22, further comprising detecting a pathogen in the sample, wherein the amount of ToBRFV in the sample is used to estimate the amount of the pathogen per the amount of the human feces in the sample.
24. The method of any one of aspects 1-23, further comprising detecting pepper mild mottle virus (PMMoV) in the sample, wherein the amount of ToBRFV in the sample in combination with the amount of PMMoV in the sample is used to estimate the amount of the pathogen per the amount of the human feces in the sample.
25. The method of aspect 24, wherein detecting PMMoV in the sample comprises further amplifying PMMoV nucleic acids using a set of primers capable of selectively amplifying at least a portion of a coat protein (CP) encoding gene of the PMMoV; and detecting the presence of the amplified PMMoV nucleic acids using a detectably labeled oligonucleotide probe sufficiently complementary to and capable of hybridizing with the PMMoV RNA or amplicon thereof, if present, as an indication of the presence or absence of the PMMoV in the sample, wherein said detecting the presence of the PMMoV in combination with the ToBRFV in the sample indicates that human feces is present in the sample.
26. The method of aspect 25, wherein the set of primers for amplifying the PMMoV nucleic acids comprises:
(a) a forward primer comprising or consisting of the sequence of SEQ ID NO:9 and a reverse primer comprising or consisting of the sequence of SEQ ID NO:10;
(b) a forward primer comprising at least 10 contiguous nucleotides of the sequence of SEQ ID NO:9 and a reverse primer comprising at least 10 contiguous nucleotides of the sequence of SEQ ID NO:10;
(c) a forward primer and a reverse primer comprising at least one nucleotide sequence that differs from the corresponding nucleotide sequence of the forward primer or reverse primer of a primer set selected from the group consisting of (a) and (b) in that the primer has up to three nucleotide changes compared to the corresponding sequence, wherein the primer is capable of hybridizing to and amplifying the PMMoV nucleic acids;
(d) a forward primer and a reverse primer comprising or consisting of nucleotide sequences that are complements of the corresponding nucleotide sequences of the forward primer and reverse primer of a primer set selected from the group consisting of (a)-(c); or
(e) a combination of a primer set selected from the group consisting of (a)-(d).
27. The method of aspect 26, wherein the oligonucleotide probe is selected from the group consisting of:
(a) a probe comprising or consisting of the sequence of SEQ ID NO:17;
(b) a probe that has up to three nucleotide changes the sequence of SEQ ID NO:17, wherein the probe is capable of hybridizing to and detecting the PMMoV RNA or amplicon thereof, and
(c) a combination of probes selected from the group consisting of (a) and (b).
28. The method of aspect 27, wherein the set of primers and the probe used for detecting the PMMoV in the sample comprises a primer comprising or consisting of the sequence of SEQ ID NO:9, a primer comprising or consisting of the sequence of SEQ ID NO:10, and a probe comprising or consisting of the sequence of SEQ ID NO:17.
29. The method of any one of aspects 1-28, further comprising performing microbial source tracking (MST), wherein the ToBRFV is used as an MST marker of human feces.
30. The method of aspect 29, wherein the ToBRFV is used to distinguish fecal contamination from a human and a non-human animal.
31. The method of aspect 30, further comprising detecting an MST marker for feces from the non-human animal.
32. The method of aspect 31, wherein the non-human animal is a wild animal, an agricultural animal, a pet, or a bird.
33. The method of aspect 32, wherein the non-human animal is a cow, dog, cat, rat, mouse, guinea-pig, goat, horse, sheep, pig, bear, deer, elk, bison, buffalo, antelope, chicken, turkey, duck, pigeon, gull, or parrot.
34. The method of any one of aspects 1-33, wherein said primers are not more than about 40 nucleotides in length.
35. The method of any one of aspects 1-34, wherein the probe is not more than about 40 nucleotides in length.
36. An isolated oligonucleotide not more than 60 nucleotides in length comprising:
(a) a nucleotide sequence comprising at least 10 contiguous nucleotides from any one of SEQ ID NOS:1-7;
(b) a nucleotide sequence having 90% sequence identity to a nucleotide sequence of (a); or
(c) complements of (a) and (b).
37. The oligonucleotide of aspect 36, further comprising a detectable label.
38. A composition comprising:
(a) a forward primer comprising or consisting of the sequence of SEQ ID NO:1 and a reverse primer comprising the sequence of SEQ ID NO:2;
(b) a forward primer comprising or consisting of the sequence of SEQ ID NO:4 and a reverse primer comprising the sequence of SEQ ID NO:5;
(c) a forward primer and a reverse primer each comprising at least 10 contiguous nucleotides from the corresponding nucleotide sequences of the forward primer and reverse primer of a primer set selected from the group consisting of (a) and (b);
(d) a forward primer and a reverse primer comprising at least one nucleotide sequence that differs from the corresponding nucleotide sequence of the forward primer or reverse primer of a primer set selected from the group consisting of (a)-(c) in that the primer has up to three nucleotide changes compared to the corresponding sequence, wherein the primer is capable of hybridizing to and amplifying ToBRFV nucleic acids in a nucleic acid amplification assay;
(e) a forward primer and a reverse primer comprising nucleotide sequences that are complements of the corresponding nucleotide sequences of the forward primer and reverse primer of a primer set selected from the group consisting of (a)-(d); or
(f) a combination of a primer set selected from the group comprising (a)-(e).
39. The composition of aspect 38, further comprising at least one oligonucleotide probe selected from the group consisting of:
(a) a probe comprising or consisting of the sequence of SEQ ID NO:3,
(b) a probe comprising or consisting of a sequence selected from the group consisting of SEQ ID NO:6 and SEQ ID NO:7,
(c) a probe that differs from the corresponding nucleotide sequence of a probe selected from the group consisting of (a) and (b) in that the probe has up to three nucleotide changes compared to the corresponding sequence, wherein the probe is capable of hybridizing to and detecting the ToBRFV RNA or amplicon thereof, and
(d) a combination of probes selected from the group comprising (a)-(c).
40. The composition of aspect 39, wherein the composition comprises:
(a) a forward primer comprising or consisting of the sequence of SEQ ID NO:1, a reverse primer comprising or consisting of the sequence of SEQ ID NO:2, and a probe comprising or consisting of the sequence of SEQ ID NO:3; and
(b) a forward primer comprising or consisting of the sequence of SEQ ID NO:4, a reverse primer comprising or consisting of the sequence of SEQ ID NO:5, and a probe comprising or consisting of the sequence of SEQ ID NO:3.
41. The composition of aspect 39 or 40, wherein said at least one probe is detectably labeled with a fluorophore.
42. The composition of aspect 41, wherein each probe is detectably labeled with a different fluorophore.
43. The composition of aspect 41 or 42, wherein each probe is detectably labeled with a 5′-fluorophore and a 3′-quencher.
44. A kit for detecting ToBRFV in a sample, the kit comprising the composition of any one of aspects 38-43 and instructions for detecting ToBRFV.
45. A method of performing wastewater surveillance of a pathogen, the method comprising:
isolating nucleic acids from a sample of wastewater comprising human feces, wherein if ToBRFV RNA is present, said nucleic acids comprise a target sequence;
amplifying the nucleic acids using a set of primers capable of selectively amplifying at least a portion of a movement protein (Mo) encoding gene or at least a portion of an RNA dependent RNA polymerase (RdRP) encoding gene;
detecting the presence of the amplified nucleic acids using a detectably labeled oligonucleotide probe sufficiently complementary to and capable of hybridizing with the ToBRFV RNA or amplicon thereof, if present, as an indication of the presence or absence of the ToBRFV in the sample of wastewater, wherein said primers and said probe are capable of selectively hybridizing to the target sequence from the ToBRFV, wherein said detecting the presence of the ToBRFV in the sample indicates that human feces is present in the sample of wastewater;
quantifying the amount of the ToBRFV RNA or amplicon thereof, wherein the amount of the ToBRFV RNA or amplicon thereof is indicative of the amount of the human feces in the sample of wastewater;
quantifying the amount of the pathogen in the sample of wastewater; and
dividing the amount of the pathogen by the amount of the human feces, as determined from said quantifying the amount of the ToBRFV RNA or amplicon thereof, to provide a normalized value for the amount of the pathogen per the amount of human feces in the wastewater.
46. The method of aspect 45, further comprising correlating the normalized value for the amount of the pathogen per the amount of human feces in the wastewater with incidence of an infectious disease caused by the pathogen.
47. The method of aspect 45 or 46, wherein the method is repeated to monitor the amount of the pathogen in the wastewater over time.
48. The method of any one of aspects 45-47, wherein said quantifying the amount of the pathogen comprises quantifying the amount of pathogen DNA, pathogen RNA, or a pathogen biomarker in the sample of wastewater.
49. The method of any one of aspects 45-48, wherein the pathogen is a virus, a bacterium, a fungus, or a parasite.
50. The method of aspect 49, wherein the virus is an enterovirus, a rotavirus, a parvovirus-like virus, an astrovirus, a calicivirus, an adenovirus, a norovirus, or a coronavirus.
51. The method of aspect 50, wherein the coronavirus is severe acute respiratory syndrome coronavirus 2 (SARS-CoV-2).
52. The method of aspect 50, wherein the enterovirus is poliovirus, coxsackie A virus, coxsackie B virus, or echovirus.
53. The method of aspect 50, wherein the virus is hepatitis A virus, hepatitis B virus, hepatitis E virus, herpesvirus, influenza A, influenza B, respiratory syncytial virus (RSV), monkeypox virus (MPXV), dengue virus, yellow fever virus, zika virus, or varicella-zoster virus.
54. The method of aspect 49, wherein the bacterium is Escherichia coli, Salmonella, Shigella, Vibrio, Campylobacter, Yersinia, or Clostridium.
55. The method of aspect 49 or 54, wherein the bacterium is an antibiotic-resistant bacterium.
56. The method of aspect 49, wherein the fungus is Candida auris, Blastomyces dermatitidis, Blastomyces gilchristii, or Cryptococcus neoformans.
57. The method of aspect 49, wherein the parasite is Entamoeba histolytica, Cryptosporidium parvum, Cyclospora cayetanensis, Giardia duodenalis, or Plasmodium falciparum.
58. The method of any one of aspects 45-57, wherein the set of primers comprises:
(a) a forward primer comprising the sequence of SEQ ID NO:1 and a reverse primer comprising the sequence of SEQ ID NO:2;
(b) a forward primer comprising the sequence of SEQ ID NO:4 and a reverse primer comprising the sequence of SEQ ID NO:5;
(c) a forward primer and a reverse primer each comprising at least 10 contiguous nucleotides from the corresponding nucleotide sequences of the forward primer and reverse primer of a primer set selected from the group consisting of (a) and (b);
(d) a forward primer and a reverse primer comprising at least one nucleotide sequence that differs from the corresponding nucleotide sequence of the forward primer or reverse primer of a primer set selected from the group consisting of (a)-(c) in that the primer has up to three nucleotide changes compared to the corresponding sequence, wherein the primer is capable of hybridizing to and amplifying the ToBRFV nucleic acids;
(e) a forward primer and a reverse primer comprising nucleotide sequences that are complements of the corresponding nucleotide sequences of the forward primer and reverse primer of a primer set selected from the group consisting of (a)-(d); or
(f) a combination of a primer set selected from the group comprising (a)-(e).
59. The method of aspect 58, wherein the set of primers used for detecting ToBRFV in the sample comprises a primer comprising the sequence of SEQ ID NO:1, a primer comprising the sequence of SEQ ID NO:2, a primer comprising the sequence of SEQ ID NO:4, and a primer comprising the sequence of SEQ ID NO:5.
60. The method of aspect 58 or 59, wherein said at least one oligonucleotide probe is selected from the group consisting of:
(a) a probe comprising the sequence of SEQ ID NO:3,
(b) a probe comprising the sequence of SEQ ID NO:6,
(c) a probe that differs from the corresponding nucleotide sequence of a probe selected from the group consisting of (a) and (b) in that the probe has up to three nucleotide changes compared to the corresponding sequence, wherein the probe is capable of hybridizing to and detecting the ToBRFV RNA or amplicon thereof, and
(d) a combination of probes selected from the group comprising (a)-(c).
61. The method of aspect 60, wherein said at least one oligonucleotide probe comprises a probe comprising the sequence of SEQ ID NO:3 and a probe comprising the sequence of SEQ ID NO:6.
62. The method of any one of aspects 45-61, said at least one probe is detectably labeled with a fluorophore.
63. The method of aspect 62, wherein each probe is detectably labeled with a different fluorophore.
64. The method of aspect 62 or 63, wherein each probe is detectably labeled with a 5′-fluorophore and a 3′-quencher.
65. The method of any one of aspects 45-64, wherein said amplifying comprises performing polymerase chain reaction (PCR) or isothermal amplification.
66. The method of aspect 65, wherein the PCR is reverse transcriptase polymerase chain reaction (RT-PCR), droplet digital polymerase chain reaction (ddPCR), or quantitative PCR (qPCR).
67. The method of aspect 66, wherein the qPCR uses a fluorogenic 5′ nuclease assay.
68. The method of aspect 65, wherein the isothermal amplification is nucleic acid sequence-based amplification (NASBA), transcription-mediated amplification (TMA), recombinase polymerase amplification (RPA), loop-mediated isothermal amplification (LAMP), strand displacement amplification (SDA), helicase-dependent amplification (HDA), nicking enzyme amplification reaction (NEAR), ligase chain reaction (LGR), or Q-beta amplification.
69. The method of any one of aspects 45-68, further comprising:
amplifying PMMoV nucleic acids using a set of primers capable of selectively amplifying at least a portion of a coat protein (CP) encoding gene of PMMoV;
detecting the presence of the amplified PMMoV nucleic acids using a detectably labeled oligonucleotide probe sufficiently complementary to and capable of hybridizing with the PMMoV RNA or amplicon thereof, if present, as an indication of the presence or absence of the PMMoV in the sample of wastewater, wherein said detecting the presence of the PMMoV in combination with the ToBRFV in the sample indicates that human feces is present in the sample of wastewater;
quantifying the amount of the ToBRFV RNA or amplicon thereof, wherein the amount of the PMMoV RNA or amplicon thereof in combination with the amount of the ToBRFV RNA or amplicon thereof is indicative of the amount of the human feces in the sample of wastewater;
quantifying the amount of the pathogen in the sample of wastewater; and
dividing the amount of the pathogen by the amount of the human feces, as determined from said quantifying the amount of the PMMoV RNA or amplicon thereof in combination with the amount of the ToBRFV RNA or amplicon thereof, to provide a normalized value for the amount of the pathogen per the amount of human feces in the wastewater.
70. The method of aspect 69, wherein the set of primers for amplifying the PMMoV nucleic acids comprises:
(a) a forward primer comprising or consisting of the sequence of SEQ ID NO:9 and a reverse primer comprising or consisting of the sequence of SEQ ID NO:10;
(b) a forward primer comprising at least 10 contiguous nucleotides of the sequence of SEQ ID NO:9 and a reverse primer comprising at least 10 contiguous nucleotides of the sequence of SEQ ID NO:10;
(c) a forward primer and a reverse primer comprising at least one nucleotide sequence that differs from the corresponding nucleotide sequence of the forward primer or reverse primer of a primer set selected from the group consisting of (a) and (b) in that the primer has up to three nucleotide changes compared to the corresponding sequence, wherein the primer is capable of hybridizing to and amplifying the PMMoV nucleic acids;
(d) a forward primer and a reverse primer comprising or consisting of nucleotide sequences that are complements of the corresponding nucleotide sequences of the forward primer and reverse primer of a primer set selected from the group consisting of (a)-(c); or
(e) a combination of a primer set selected from the group consisting of (a)-(d).
71. The method of aspect 70, wherein the oligonucleotide probe is selected from the group consisting of:
(a) a probe comprising or consisting of the sequence of SEQ ID NO:17;
(b) a probe that has up to three nucleotide changes the sequence of SEQ ID NO:17, wherein the probe is capable of hybridizing to and detecting the PMMoV RNA or amplicon thereof, and
(c) a combination of probes selected from the group consisting of (a) and (b).
72. The method of aspect 71, wherein the set of primers and the probe used for detecting the PMMoV in the sample comprises a primer comprising or consisting of the sequence of SEQ ID NO:9, a primer comprising or consisting of the sequence of SEQ ID NO:10, and a probe comprising or consisting of the sequence of SEQ ID NO:17.
73. The method of any one of aspects 45-72, further comprising amplifying the nucleic acids using a set of primers capable of selectively amplifying at least a portion of a SARS-CoV-2 envelope (E) encoding gene and a nucleocapsid protein N2 (N2) encoding gene;
detecting the presence of the amplified nucleic acids using a detectably labeled oligonucleotide probe sufficiently complementary to and capable of hybridizing with the SARS-CoV-2 RNA or amplicon thereof, if present, as an indication of the presence or absence of the SARS-CoV-2 in the sample of wastewater;
quantifying the amount of the SARS-CoV-2 RNA or amplicon thereof, wherein the amount of the SARS-CoV-2 RNA or amplicon thereof is indicative of the amount of the SARS-CoV-2 in the sample of wastewater; and
dividing the amount of the SARS-CoV-2 RNA or amplicon thereof by the amount of the ToBRFV RNA or amplicon thereof, to provide a normalized value for the amount of the SARS-CoV-2 per the amount of human feces in the wastewater.
74. The method of aspect 73, wherein the set of primers for amplifying the SARS-CoV-2 nucleic acids comprises:
(a) a forward primer comprising or consisting of the sequence of SEQ ID NO:13 and a reverse primer comprising or consisting of the sequence of SEQ ID NO:14;
(b) a forward primer comprising or consisting of the sequence of SEQ ID NO:15 and a reverse primer comprising or consisting of the sequence of SEQ ID NO:16;
(c) a forward primer and a reverse primer each comprising at least 10 contiguous nucleotides from the corresponding nucleotide sequences of the forward primer and reverse primer of a primer set selected from the group consisting of (a) and (b);
(d) a forward primer and a reverse primer comprising at least one nucleotide sequence that differs from the corresponding nucleotide sequence of the forward primer or reverse primer of a primer set selected from the group consisting of (a)-(c) in that the primer has up to three nucleotide changes compared to the corresponding sequence, wherein the primer is capable of hybridizing to and amplifying the SARS-CoV-2 nucleic acids;
(e) a forward primer and a reverse primer comprising or consisting of nucleotide sequences that are complements of the corresponding nucleotide sequences of the forward primer and reverse primer of a primer set selected from the group consisting of (a)-(d); or
(f) a combination of a primer set selected from the group consisting of (a)-(e).
75. The method of aspect 74, wherein the oligonucleotide probe is selected from the group consisting of:
(a) a probe comprising or consisting of the sequence of SEQ ID NO:19,
(b) a probe comprising or consisting of the sequence of SEQ ID NO:20,
(c) a probe that differs from the corresponding nucleotide sequence of a probe selected from the group consisting of (a) and (b) in that the probe has up to three nucleotide changes compared to the corresponding sequence, wherein the probe is capable of hybridizing to and detecting the SARS-CoV-2 RNA or amplicon thereof, and
(d) a combination of probes selected from the group consisting of (a)-(c).
76. The method of aspect 75, wherein the set of primers and probes used for detecting the SARS-CoV-2 RNA in the sample comprises:
a primer comprising or consisting of the sequence of SEQ ID NO:13, a primer comprising or consisting of the sequence of SEQ ID NO:14, and a probe comprising or consisting of the sequence of SEQ ID NO:19; and
a primer comprising or consisting of the sequence of SEQ ID NO:15, a primer comprising or consisting of the sequence of SEQ ID NO:16, and a probe comprising or consisting of the sequence of SEQ ID NO:20.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### Example 1

### Tomato Brown Rugose Fruit Virus Yields Single-Strand Positive-Sense RNA as a Novel Microbial Source Tracking Marker

### Introduction

The process of detecting microbes and identifying sources of microbial contamination in the environment is known as microbial source tracking (MST). MST targets have also been used in severe acute respiratory syndrome coronavirus 2 (SARS-CoV-2) wastewater-based epidemiology applications as “fecal strength” and endogenous extraction controls (8). Over the last decade, sensitive and specific molecular MST markers have been developed for various animal stools, including those from humans (9), cows (10), and birds (11). Most of these MST markers target conserved regions of bacterial genomes (9), with the exception of two that target viruses, the cross-assembly phage (crAssphage) (12) and pepper mild mottle virus (PMMoV) (13). crAssphage, a phage of Bacteroidetes, is a DNA virus that is highly abundant in human stool (14). PMMoV is a plant RNA virus found at high concentrations in human stool given its presence in popular spices, hot sauces, and other food products (15). The performance of MST targets is evaluated in terms of sensitivity and specificity for a given host's stool. For instance, a sensitive target for human stool is present at high concentrations in nearly all human fecal samples, so that dilute human stool can be detected in the environment. Meanwhile, a specific target is absent in nearly all non-human fecal samples. A previous study defined an MST assay as being sensitive and specific if the true positive and true negative rates were greater than 80% (9).

In this study, we present a new human-associated, RNA-based, viral MST target that is highly abundant in human stool and wastewater, tomato brown rugose fruit virus (ToBRFV). ToBRFV was first identified in Israel in 2014 and has since been detected across the world. As of early 2023, ToBRFV had been found across four continents, in at least 35 countries; this is likely an underestimate (16). We assembled eight nearly complete genomes of ToBRFV from wastewater and stool samples from the San Francisco Bay Area (Bay Area) in California in the United States, representing some of the first complete genomes from stool and wastewater in the area. Using these complete genomes and other publicly available genomes, we developed two novel hydrolysis probe-based reverse transcription-PCR (RT-PCR) assays based on conserved regions of its RNA genome and tested their sensitivity and specificity using stool and wastewater samples. Finally, we used this assay for MST in storm-water samples collected from an urban environment. With the finding that ToBRFV is a reliable RNA-virus based MST marker, this study makes a valuable contribution to detecting human fecal contamination of the environment and to wastewater-based epidemiology.

### Results and Discussion

ToBRFV is widely prevalent and abundant in sequence data from stool and wastewater samples. Tracking the presence of human feces in the environment and identifying internal controls for the processing of stool and wastewater samples require marker genes that are (i) prevalent, i.e., consistently present across samples, and (ii) abundant, i.e., at high enough concentration for reliable detection. crAssphage (12) is one such DNA-based marker, and PMMoV is an RNA-based marker (13). We sought to identify the most abundant and prevalent source of RNA from RNA sequencing data from human stool and wastewater samples.

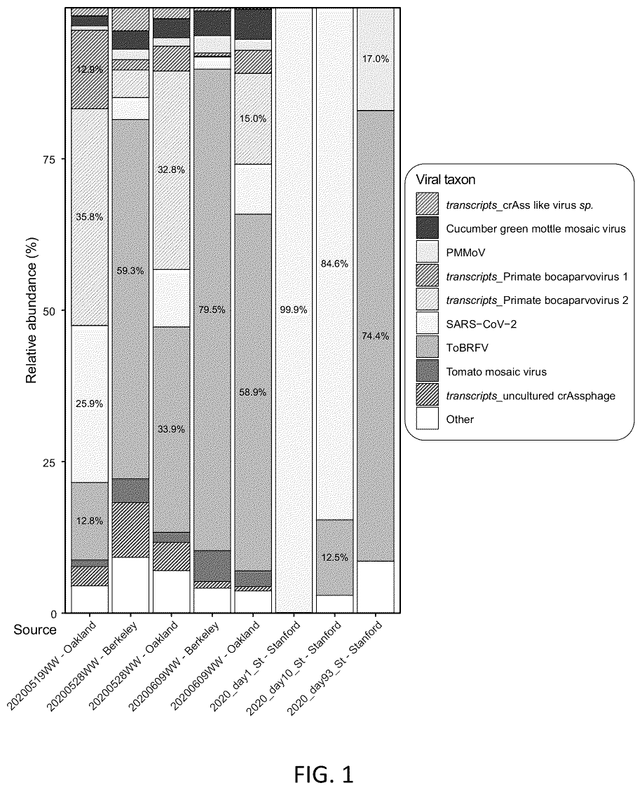

We isolated and sequenced RNA from three longitudinal stool samples from one human participant who had tested positive for SARS-CoV-2. In parallel, we acquired publicly available transcriptomics data from five wastewater samples that had been collected and sequenced from the Bay Area (17). Using these sequence data from eight samples, we identified all represented RNA viruses and their relative abundances (FIG. 1). ToBRFV was the most widely prevalent RNA virus, present in all five wastewater samples and three stool samples. It was detected at very low relative abundance (0.077% of viral reads) in one of the stool samples during the time of active SARS-CoV-2 infection, in which 99.9% of viral reads belonged to SARS-CoV-2. In the other seven samples where it was detected, it was the only viral RNA with a relative abundance consistently over 10.0% in viral reads, often making up over 50.0% of the reads. Notably, the relative abundance of ToBRFV was consistently greater than that of PMMoV, which is a well-established MST marker and known to be highly abundant in wastewater (8). This is consistent with reports from studies carried out prior to (17) and in parallel (18, 19) with ours that also show that ToBRFV is a highly prevalent virus in wastewater.

Novel ToBRFV genomes and sequence analysis reveal suitable RNA-borne marker genes. Having determined that ToBRFV is a prevalent and abundant RNA virus in sequence data, we next set out to identify genomic regions suitable as targets for primers/probes for its reliable molecular detection.

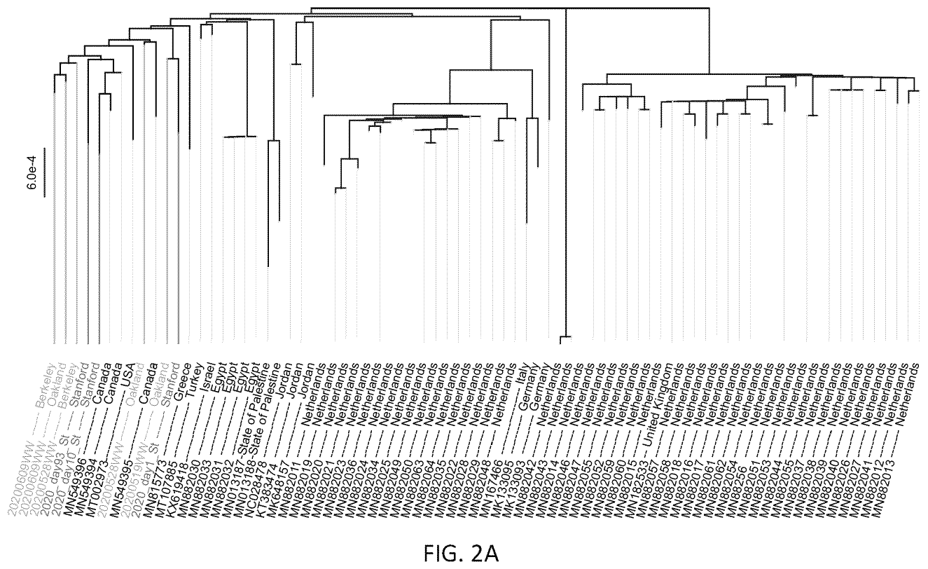

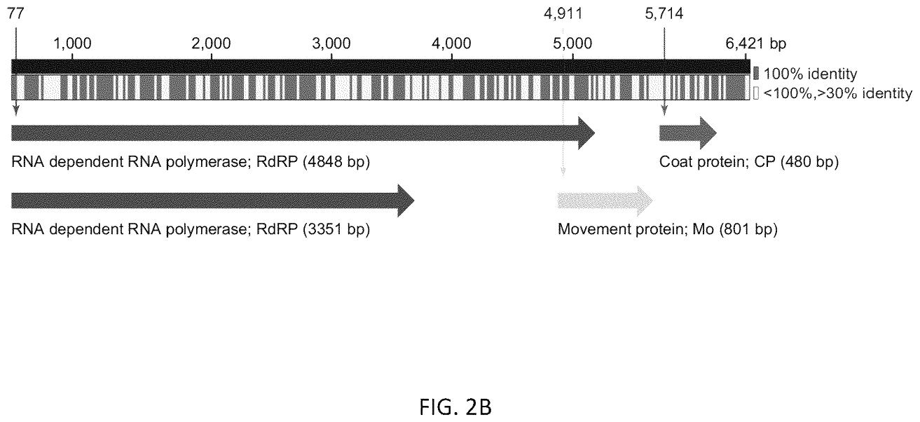

In February 2021, at the start of this study, only 70 nearly complete ToBRFV genomes were known. Fifty of these were from the Netherlands. None had been sequenced from human stool or wastewater samples, and only one sequence was from the United States. In order to ensure that the assay we developed was universal, we first decided to augment the number of ToBRFV genomes and the diversity of their sources. Therefore, we assembled nearly complete genomes of ToBRFV using sequence data generated in this study from stool samples and using existing data from wastewater samples (17), both collected in the Bay Area. The eight newly assembled genomes had a mean completeness of 98.8% (range, 93.6% to 100.0%; median, 99.4%) (see Table S5 in the supplemental material). The longitudinally acquired stool samples yielded ToBRFV genomes with single nucleotide polymorphisms (SNPs) in 27 positions, suggesting possible strain variation over time. Looking more broadly, across all 78 nearly complete ToBRFV genomes, we identified 2,808 positions containing SNPs (across an average contig length of 6,366 bp), and the 12 North American strains form their own distinct cluster (FIG. 2B).

Multiple-sequence analysis across all 78 ToBRFV genomes highlights regions that are 100.0% conserved (FIG. 2C). Among these, gene annotation reveals (i) two variants of the RNA-dependent RNA polymerase (RdRP)-encoding gene at 2,700 bp on the chromosome, which differ by whether an internal stop codon is read through (size, 3,351 bp or 4,848 bp), (ii) the movement protein (Mo)-encoding gene (size, 480 bp) at 5,166 bp, and (iii) the coat protein (CP)-encoding gene (size, 801 bp) at 5,166 bp (FIG. 2C). Among these, we designed primer/probe sets targeting the 59 end of the RdRP gene and the Mo gene. We were unable to identify a suitable primer set for the CP gene for droplet digital RT-PCR (ddRT-PCR). Notably, the primer/probe sets designed here (Table 1) were conserved across all 78 genomes (FIG. 2D).

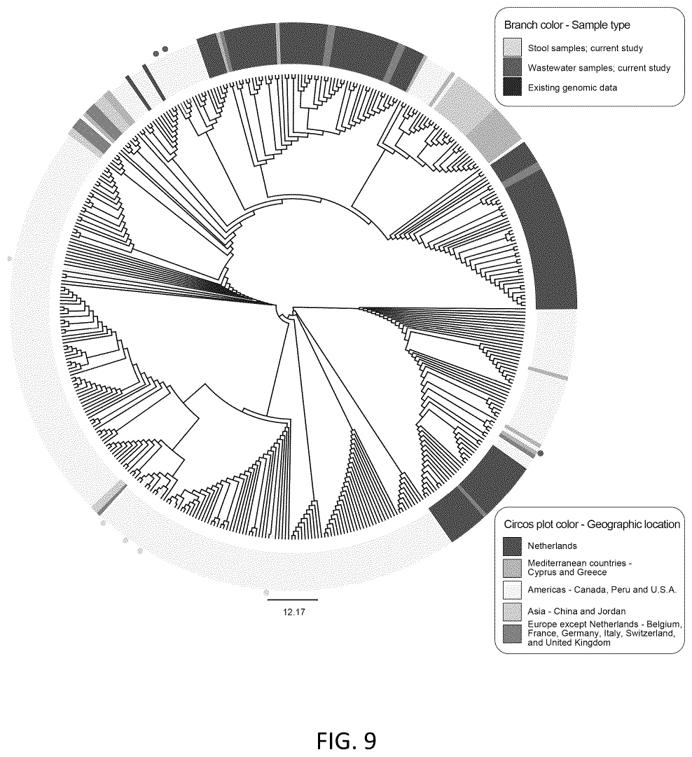

Between the first phase of this study in February 2021 and the completion of this work in November 2022, the number of nearly complete ToBRFV genomes increased to 441 (Table 2), with additional genomes from Belgium, France, Mexico, Switzerland, and the United States. Therefore, we repeated the phylogenetic analysis of the novel genomes generated in the current study in the context of all 441 currently known genomes (FIG. 9). Again, we found that the genomes derived from North America cluster distinctly. Finally, we analyzed whether the primer/probe sets proposed here continue to be universal and found that the oligonucleotides targeting Mo are a perfect sequence match in 439/441 genomes, while those targeting RdRP are a perfect match in 436/441 genomes (FIG. 2C).

ToBRFV-targeting primer/probe sets have low limit of blank and limit of detection. Having newly designed primer/probe sets targeting the Mo and RdRP genes in ToBRFV, we aimed to validate these oligonucleotides and establish the limits of their reliable utility.

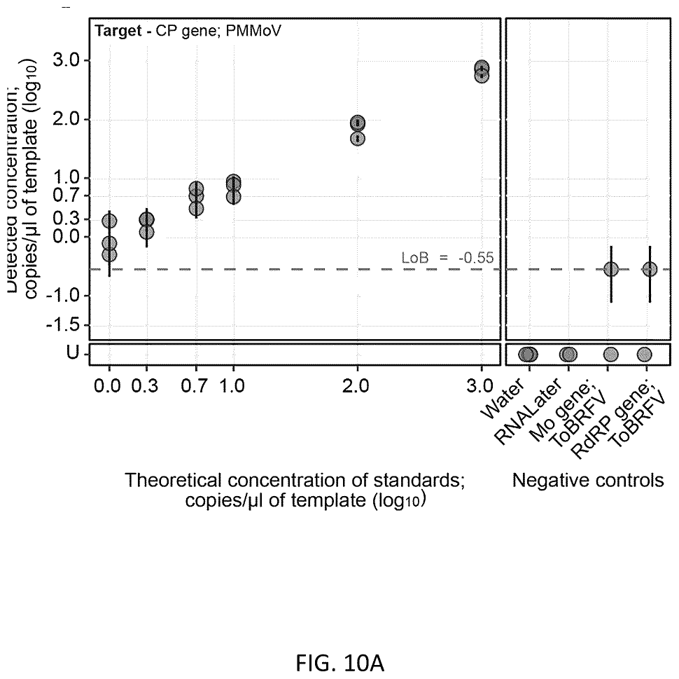

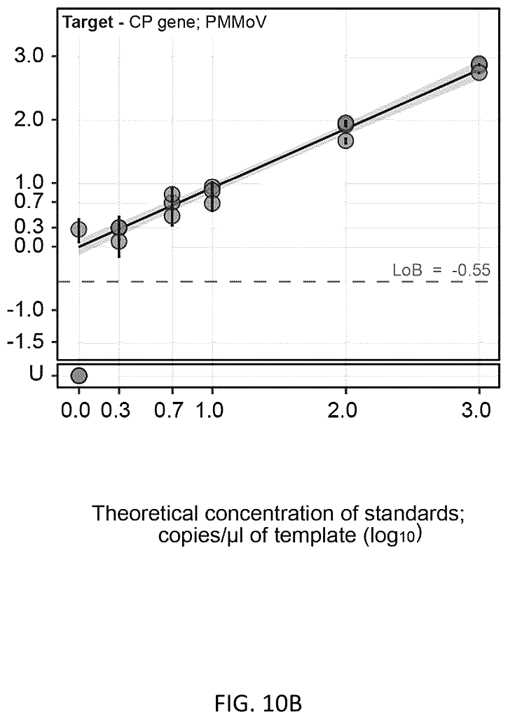

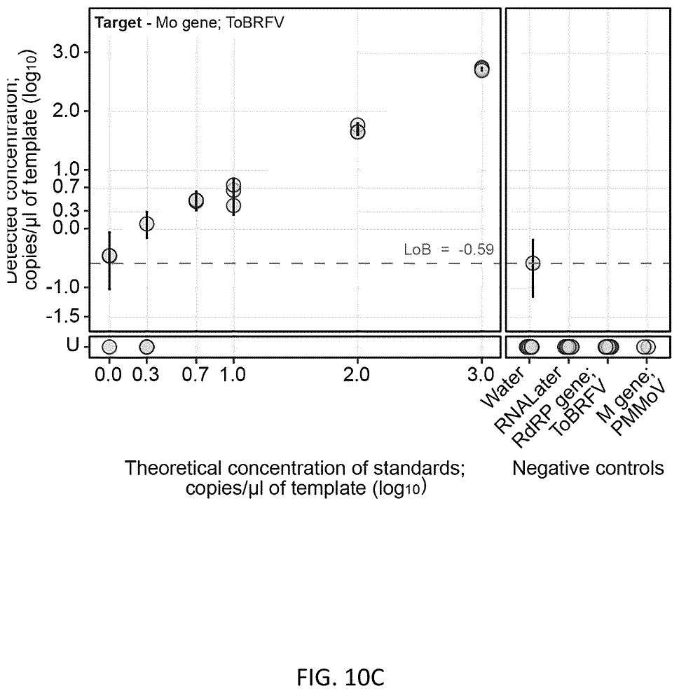

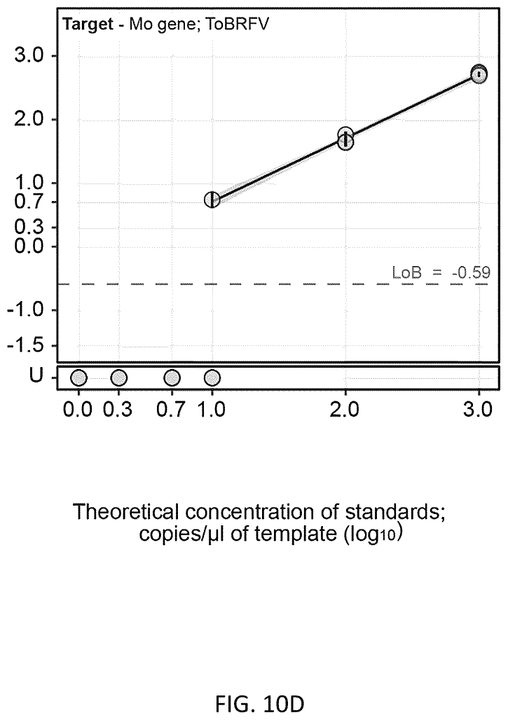

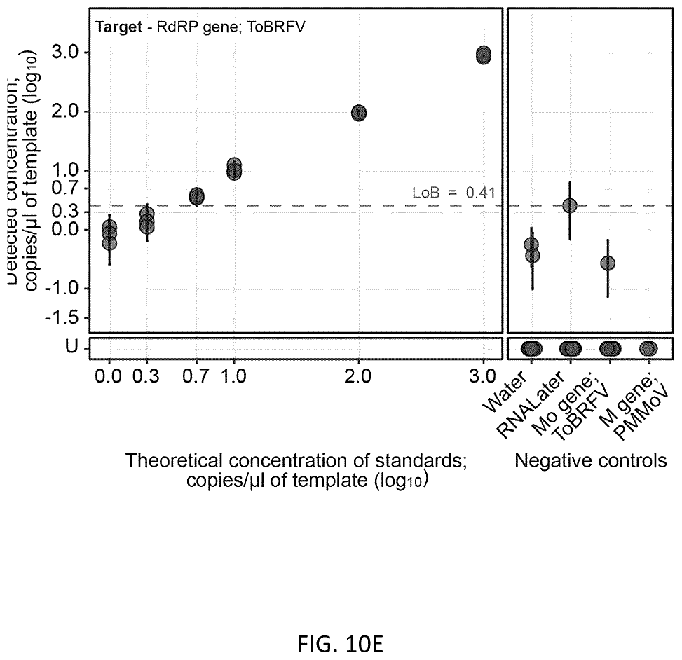

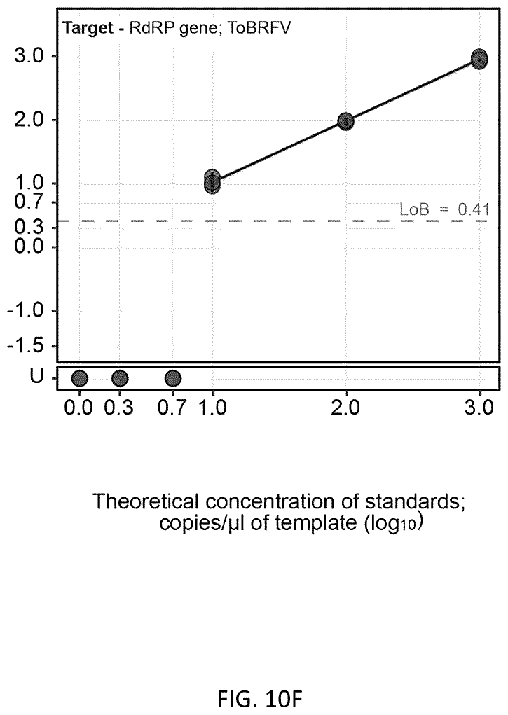

To this end, we acquired synthetic DNA constructs featuring regions of the ToBRFV Mo and RdRP genes targeted by hydrolysis-probe RT-PCR assays from Integrated DNA Technologies (IDT) cloned into the pIDT plasmid. We also acquired a similar plasmid containing the PMMoV CP gene. Using ddRT-PCR, we assayed a dilution series of these synthetic plasmid constructs at 1, 2, 5, 10, 100, and 1,000 copies/mL of template in triplicate and found that all the primer/probe sets detected the target gene at all concentrations (FIG. 10). Next, we focused our attention on the negative controls included in the assays to identify the limit of detection (LoD) for each primer/probe set. The negative controls included two no-template controls, water and RNAlater, and two mis-matched controls that were the synthetic pIDT plasmids bearing targets orthogonal to the primer/probe sets. Therefore, theoretically, all the negative controls would have no detectable gene target. For each primer/probe set, among the negative controls, we identified the highest concentration of target detected and set this value as the limit of blank (LoB). This means that any concentration below 20.552 log₁₀ copies/mL of template for the primer/probe set targeting PMMoV CP gene, 20.590 log₁₀ copies/mL of template for the ToBRFV Mo gene, and 0.407 log₁₀ copies/mL of template for the ToBRFV RdRP gene is not reliable (FIG. 10). After converting all concentrations of gene targets below the LoB to zero, we focused our attention on the triplicate dilution series to identify the lowest concentration of template at which all three reactions had a detectable target concentration (FIG. 10). We set this concentration as the LoD, i.e., the lowest concentration at which a gene target can be reliably detected. The LoD for the primer/probe set targeting the PMMoV CP gene was 1 copy/mL of template, that for the ToBRFV Mo gene was 5 copies/mL of template, and that for the ToBRFV RdRP gene was 5 copies/mL of template. All gene target concentrations below the LoD were set to zero.

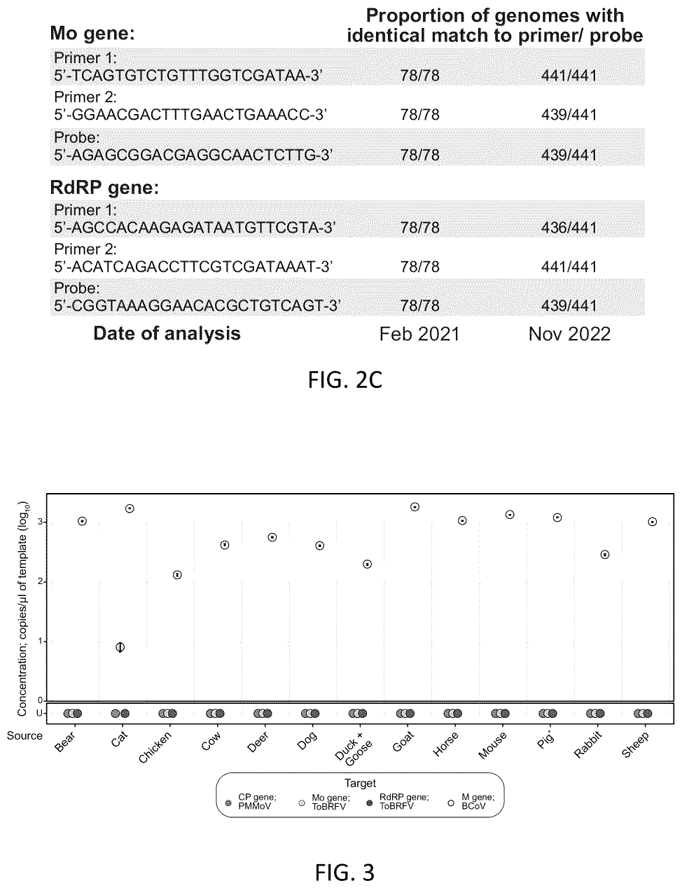

ToBRFV was not detected in stool from non-human animals. MST targets should be specific, meaning that they are mostly absent in stool from other common animals. Therefore, having established that our primer/probe sets are functional, we tested them against stool collected from 14 different animals, including wild bear and deer, chickens, cows, ducks, geese, goats, and sheep from a farm, horses and pigs from a barn, a household cat, dog, and rabbit, and laboratory mice. Notably, these animals are rather diverse and are fed a wide variety of foods. While RNA extracted from all of these animal samples had a detectable concentration of the M gene target from the spiked-in bovine coronavirus (BCoV) used as a control, none of them had RNA containing either the PMMoV CP gene or the ToBRFV RdRP gene (FIG. 3). The ToBRFV Mo gene was detectable only in the sample derived from the domesticated cat, perhaps due to inclusion of tomatoes in its processed kibble or cross contamination of its diet with that of its human cohabitant. Therefore, all three primer/probe sets to detect RNA from PMMoV and ToBRFV do not detect RNA in most animal feces, except for the ToBRFV Mo gene in a cat, indicating that they are specific for human stool.

To test whether the absence of PMMoV and ToBRFV gene targets in these samples was an artifact of inhibited RT-PCR, we diluted the RNA extracts 1:10 and assayed for the gene targets from PMMoV and ToBRFV. Results obtained with the diluted template were the same as those obtained with undiluted template, indicating the absence of inhibition, with one exception. In the assay for the PMMoV CP gene, we found that the diluted template from pig's stool yielded a detectable concentration of 7.47 log₁₀ copies/mL of template, suggesting that this animal may have ingested some PMMoV as part of its diet and that the corresponding assay with the undiluted template was affected by PCR inhibition.

Description of participants who provided human stool samples used for RNA quantification. Analyzing sequence information from three stool samples collected from one human participant revealed ToBRFV to be abundantly present. To further test the sensitivity of the assays to human stool, we relied on a stool biobank including 194 stool samples from 125 adults and 28 samples from four children, all of whom were undergoing hematopoietic cell transplantation (HCT), cell therapy (CAR-T [chimeric antigen receptor T cell]), or induction chemotherapy for the treatment of underlying hematologic disorders.

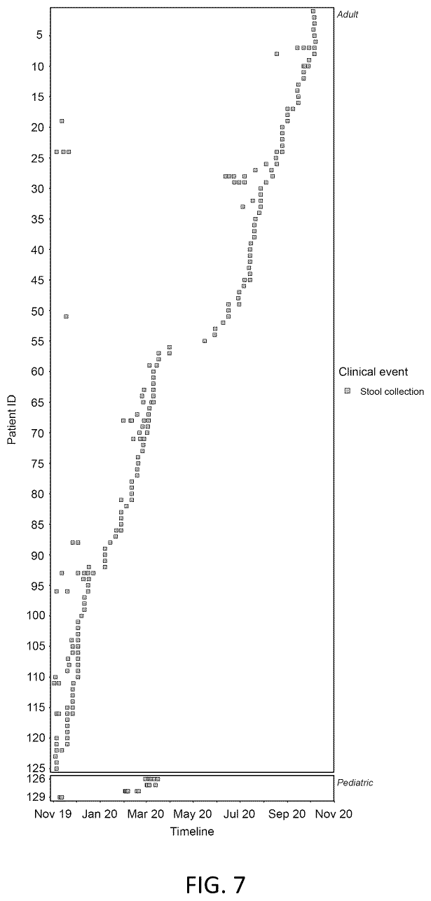

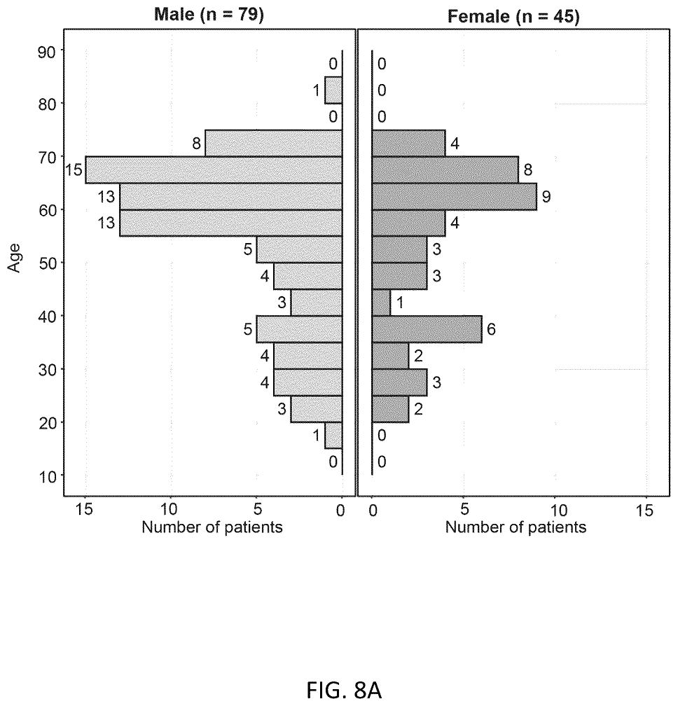

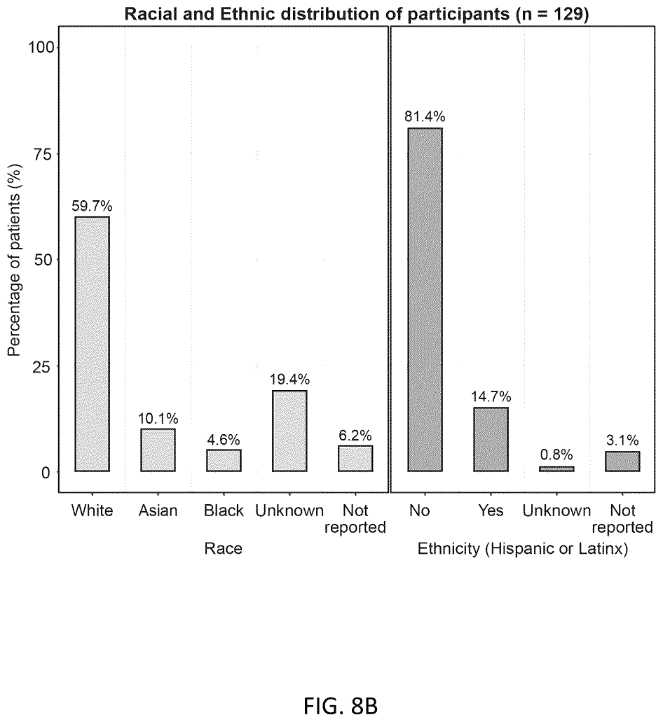

Of the adult participants, 79 were male, 45 were female, and 1 did not provide information on their sex. The median age of the adult participants was 60 years (range, 19 to 82 years), and that of the pediatric participants was 6 years (range, 3 to 16 years). Among the adult participants, 61.6% self-identified as white. Age, race, and ethnicity information on pediatric participants is withheld, since it can be used to identify the participants. The timeline of stool collection is summarized in FIG. 7. Demographic information is summarized in FIG. 8 and Table S1.

ToBRFV is more prevalent in human stool samples than PMMoV. We tested whether RNA extracted from human stool samples was susceptible to RT-PCR inhibition. We assayed eight randomly selected RNA extracts for all three targets, the ToBRFV RdRP gene, the PMMoV CP gene, and the ToBRFV Mo gene, using both 1:10-diluted and undiluted templates. No RT-PCR inhibition was detected in the assay for the ToBRFV RdRP gene, since both diluted and undiluted templates provided the same results. However, in the assays for the PMMoV CP gene and the ToBRFV Mo gene, the diluted templates provided a higher concentration of the gene targets in one and two of the eight samples, respectively, indicating inhibition of the corresponding RT-PCRs. Since inhibition of RT-PCR was observed infrequently, in far less than 50% of the reactions, we assayed all of the samples in their undiluted format to retain higher sensitivity.

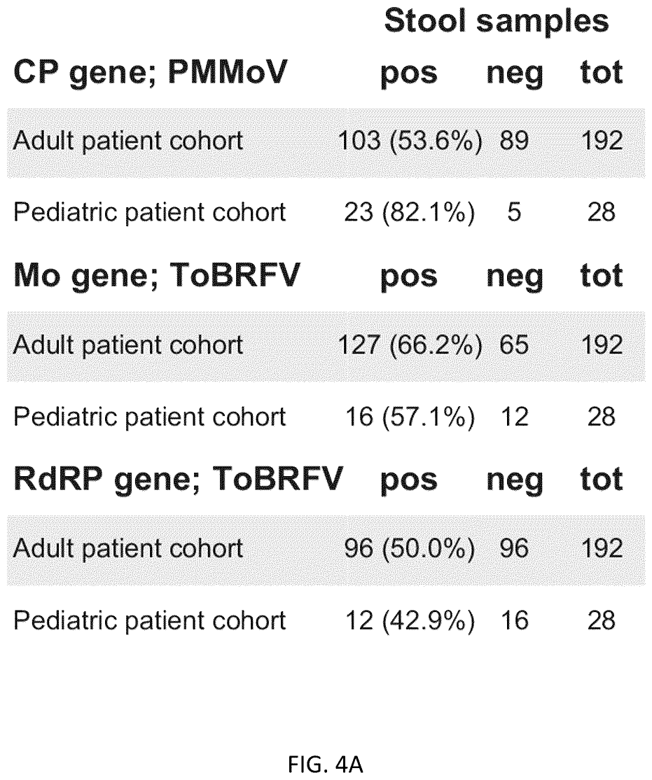

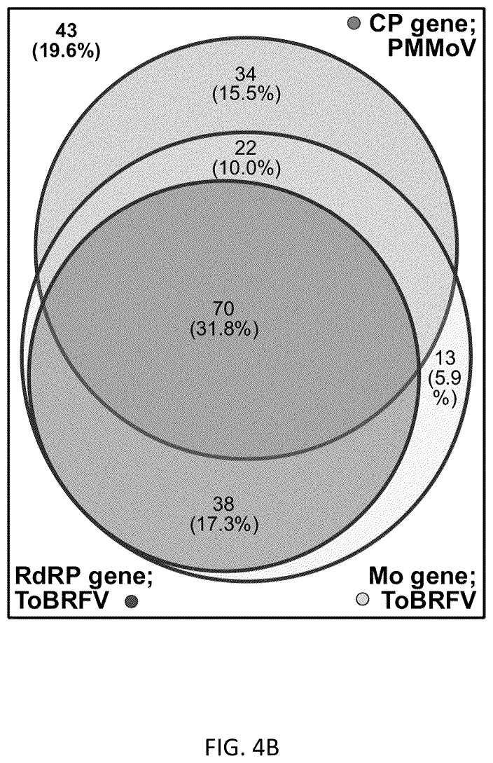

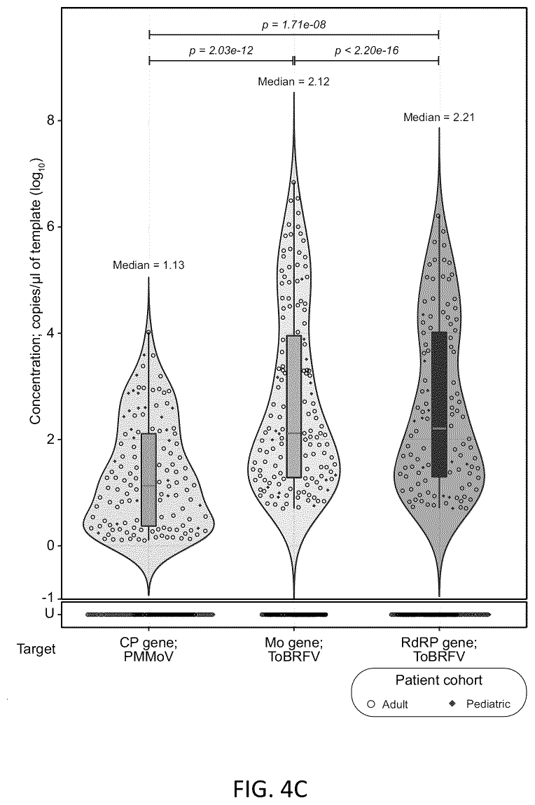

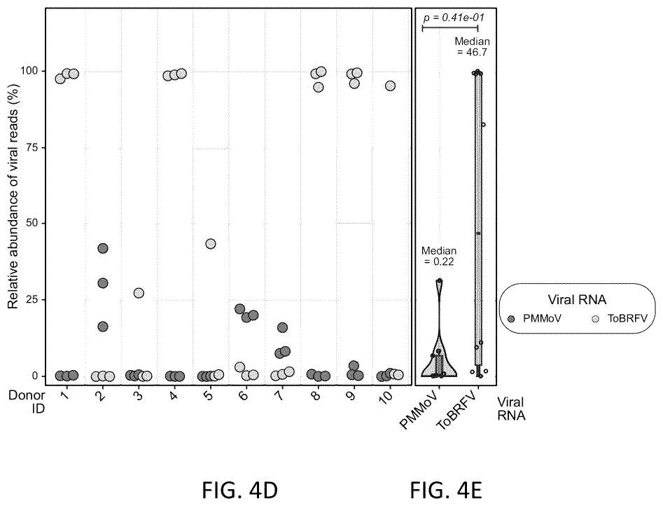

Of 222 RNA extracts derived from 129 participants, 220 had detectable BCoV RNA. This suggests that two of the RNA extractions failed; those samples were therefore excluded from further analysis, altering our study cohort size to 127 (123 adult; 4 pediatric). Among the remaining stool samples, 126/220 (57.3%) had detectable levels of the PMMoV CP gene, while 143/220 (65.0%) had the ToBRFV Mo gene and 108/220 (49.1%) had the ToBRFV RdRP gene; the ToBRFV Mo gene was the most prevalent target gene. This prevalence varied in the two patient cohorts (FIG. 4A); 127/192 (66.2%) stool samples from adult participants had detectable amounts of the ToBRFV Mo gene, more than in the case of PMMoV CP gene (103/192; 54.7%), but only 16/28 (57.1%) stool samples from pediatric patients had detectable amounts of the ToBRFV Mo gene, fewer than in the case of PMMoV CP gene (23/28; 82.1%).

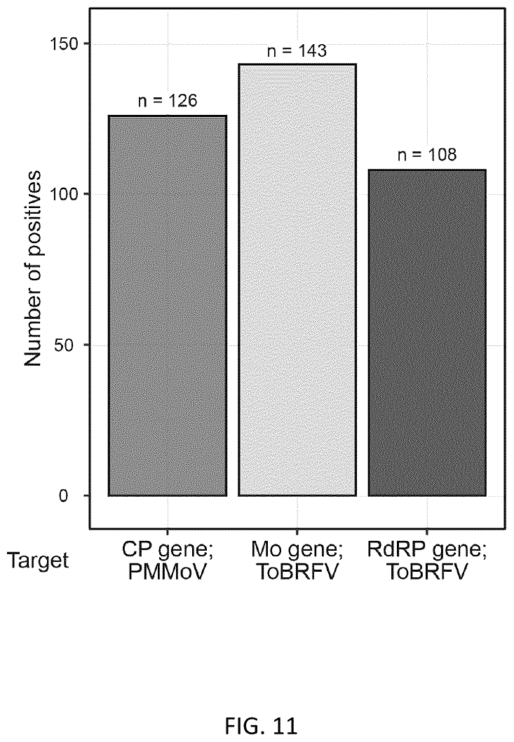

In analyzing the prevalence of the three gene targets of interest in the stool samples, we detected all three gene targets in 70 (31.8%) of the samples, while we detected none of the three gene targets in 43 (19.6%) (FIG. 4B; FIG. 11). Notably, in 34 (15.5%) of the samples, we detected only the PMMoV CP gene, and in 13 (5.9%), we detected only the ToBRFV Mo gene. In all samples in which we detected the ToBRFV RdRP gene, we also detected the ToBRFV Mo gene. This analysis suggests that while the ToBRFV Mo gene is the most prevalent RNA-based marker of human stool, combining this with the detection of the PMMoV CP gene will provide the most coverage, more than 80.0% of stool samples.

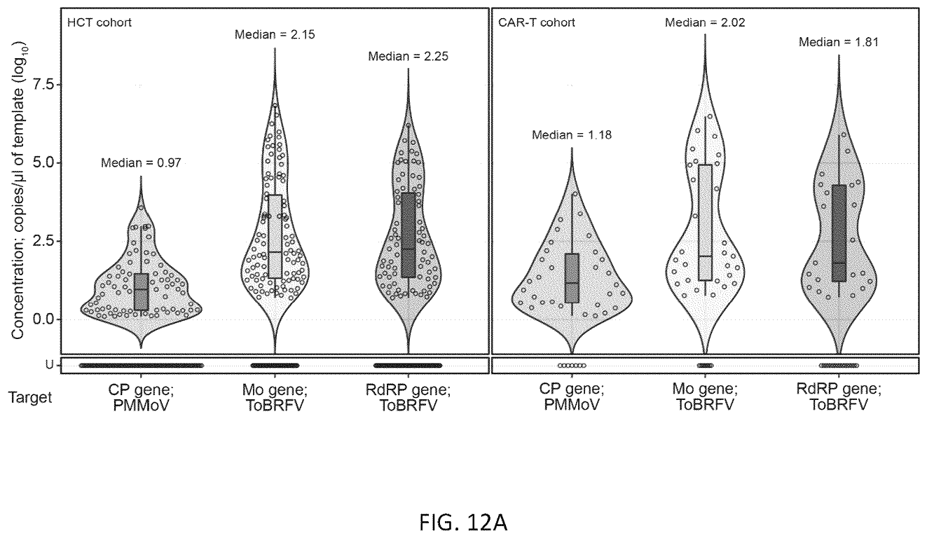

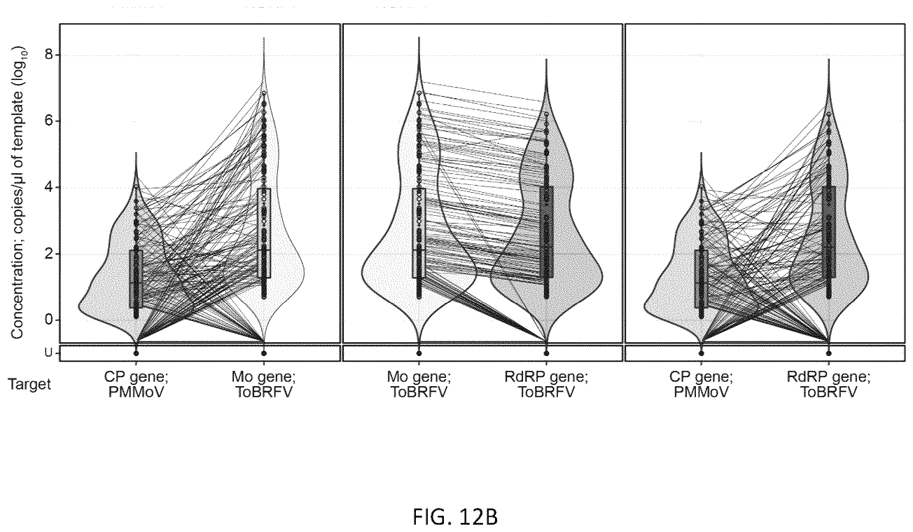

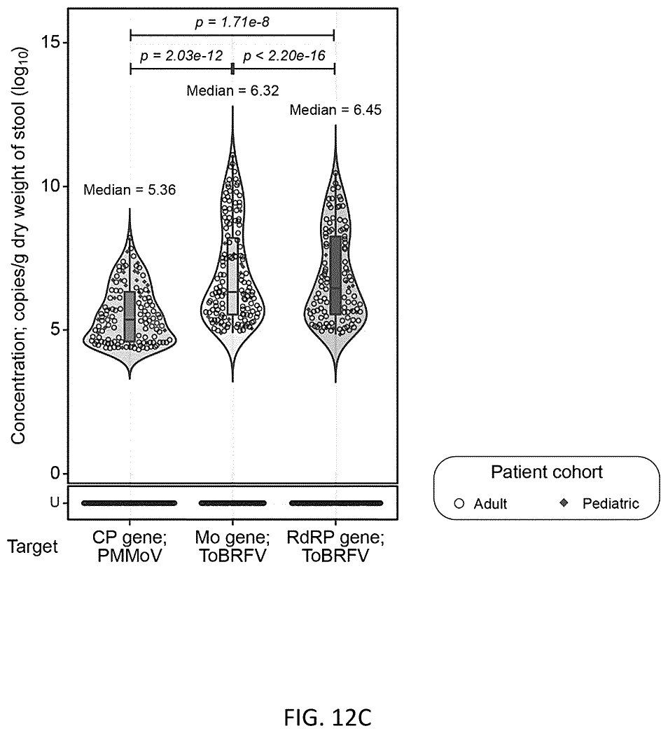

Next, we analyzed the abundance of each of these gene targets in stool samples. The median detected concentration of the PMMoV CP gene is lower than that of the ToBRFV Mo gene (1.13 log₁₀ copies/mL of template versus 2.12 log₁₀ copies/mL of template; Wilcoxon signed rank test P=2.03e212) and the ToBRFV RdRP gene (1.13 log₁₀ copies/mL of template versus 2.21 log₁₀ copies/mL of template; P=1.17e28) (FIG. 4C). These stool samples were derived from participants undergoing different treatments for underlying hematologic disorders. Therefore, we investigated whether the nature of treatment was a confounding factor. Here, again, we found that the median abundances of both target genes from ToBRFV are higher than that of the PMMoV CP gene, even when the samples were separated by treatment cohort (FIG. 12A). Further, a paired comparison of target gene abundances validates the previous observation that all samples that tested positive for the ToBRFV RdRP gene also tested positive for the ToBRFV Mo gene (FIG. 12B).

While the concentration of the various gene targets has so far been reported in copies per microliter of template, we recognize that studies also measure molecular targets in units per gram (dry weight) of stool sample. Therefore, we chose five samples per cohort at random, dried two biopsy punches from each sample, and found that the mean percent (dry weight) in the samples from adults undergoing HCT treatment was 23.6% (range, 18.2 to 33.9%), that for adults undergoing CAR-T treatment was 27.5% (range, 22.2 to 31.6%), and that of pediatric patients undergoing induction chemotherapy was 32.4% (range, 23.8 to 40.3%). We used the average percent dry weight to convert gene target concentrations to copies per gram (dry weight) of stool samples in FIG. 12C. In brief, the median concentrations of ToBRFV RdRP and Mo genes were 6.45 and 6.32 log₁₀ copies/g (dry weight) of stool, and that of the PMMoV CP gene was 5.36 log₁₀ copies/g (FIG. 12C).

To determine if our findings are generalizable to applications beyond a cohort of patients, we looked at an alternate data set recently generated in our group that sequenced RNA from stool collected from 10 healthy individuals in triplicate and frozen (20). In this data set also, the relative abundance of ToBRFV was consistently greater than that of PMMoV, as reflected by their median relative abundances of 46.7 versus 0.22% viral RNA reads (FIG. 4D). Taken together, these observations indicate that the abundance of ToBRFV is greater than that of PMMoV in human stool samples, and the ToBRFV Mo gene may thus be preferable to the PMMoV CP gene as an MST marker.

The ToBRFV Mo gene is prevalent and abundant in wastewater samples. Wastewater is a complex matrix containing human stool and other biological excretions, in addition to food waste, industrial waste, and infiltrating stormwater in some cases. We next validated the molecular detection test developed here for testing this sample type. We acquired wastewater solid samples from 15 cities in the United States, extracted RNA, and assayed it for the presence and abundance of the gene targets of interest.

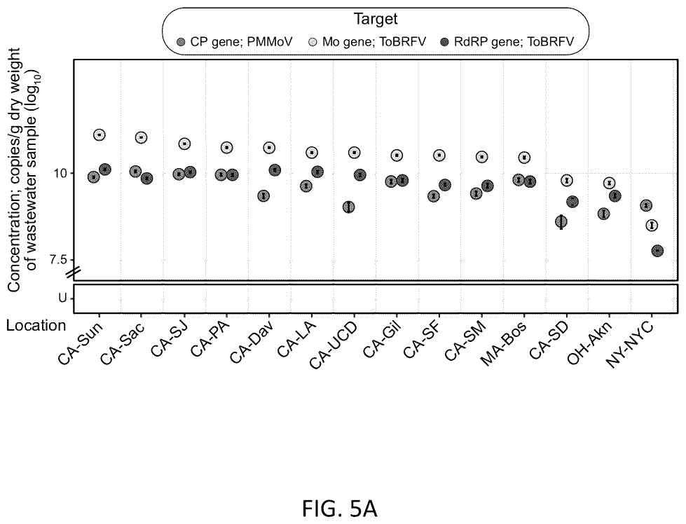

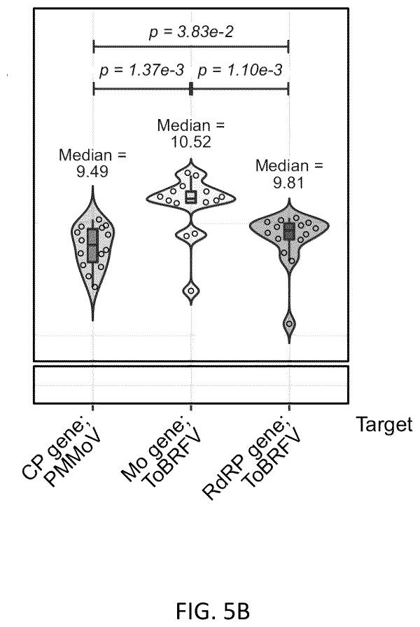

The extracted RNA from Wisconsin did not have detectable amounts of any of the gene targets; this matches unpublished data generated using this sample by a different group, and this RNA was excluded from further analysis, reducing our sample size to 14. Thirteen of these samples had more ToBRFV Mo gene than the other two molecular markers, with the sample from New York being the exception, having the PMMoV CP gene in the highest concentration (FIG. 5A). Looking at the data in aggregate, the samples had a median concentration of 10.5 log₁₀ copies/g (dry weight) of wastewater solids (standard deviation of 0.67 and interquartile range [IQR] of 0.26 log₁₀ copies/g) of the ToBRFV Mo gene, followed by 9.81 log₁₀ copies/g (standard deviation of 0.60 and IQR of 0.36 log₁₀ copies/g) of the ToBRFV RdRP gene and 9.49 log₁₀ copies/g (standard deviation of 0.46 and IQR of 0.74 log₁₀ copies/g) of the PMMoV CP gene. Pairwise comparison of gene target concentrations across samples using the Wilcoxon signed-rank test revealed that the increased detection of the ToBRFV Mo gene is statistically significant in comparison to detection of the PMMoV CP gene (P=1.37e23) and the ToBRFV RdRP gene (P=1.10e23).

Our analytical workflow to purify RNA from wastewater samples has previously been shown to yield templates free of RT-PCR inhibitors (21). Additionally, we diluted RNA extracts from wastewater samples 1:10,000 prior to use as templates in ddPCR assays to detect the ToBRFV and PMMoV gene targets. This high dilution further mitigates the likelihood of RT-PCR inhibition.

The ToBRFV Mo gene matches crAssphage ORF000024 as an indicator of fecal contamination of stormwater. crAssphage ORF000024 is a well-established human-associated microbial source tracking marker (12). We compared concentrations of PMMoV and ToBRFV RNA targets to those of this crAssphage DNA target in stormwater draining from urbanized watersheds in the Bay Area. crAssphage ORF000024 was previously quantified in these samples and was reported by Graham et al. (22).

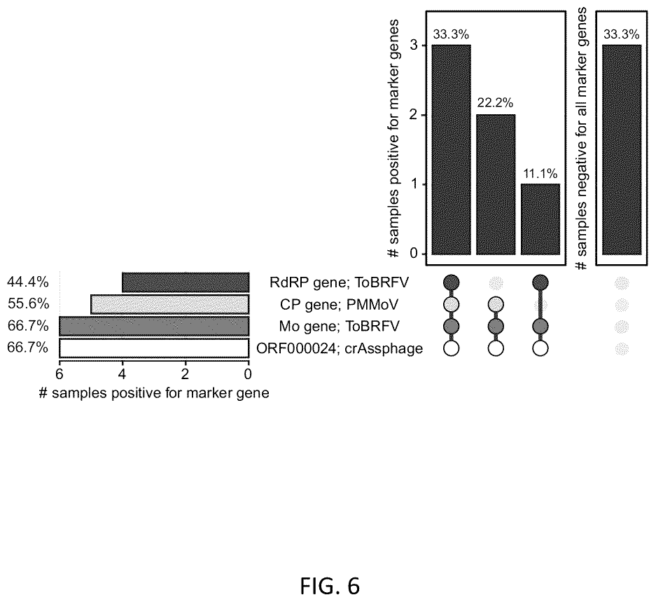

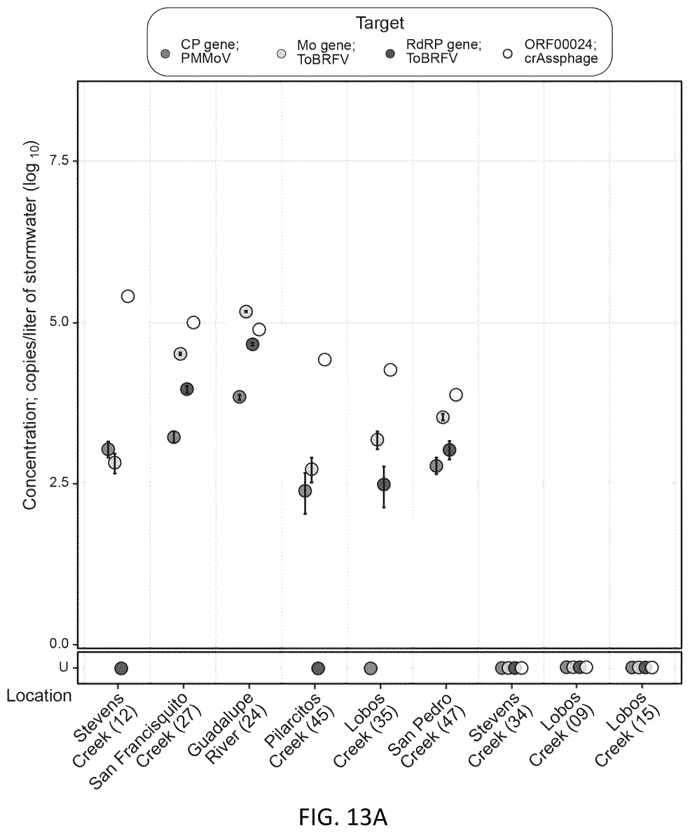

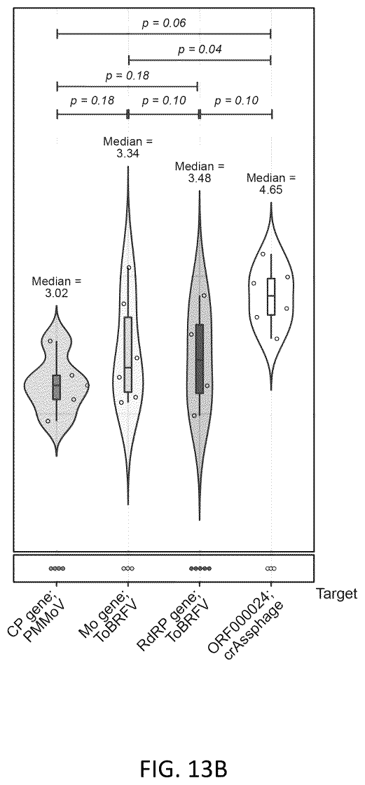

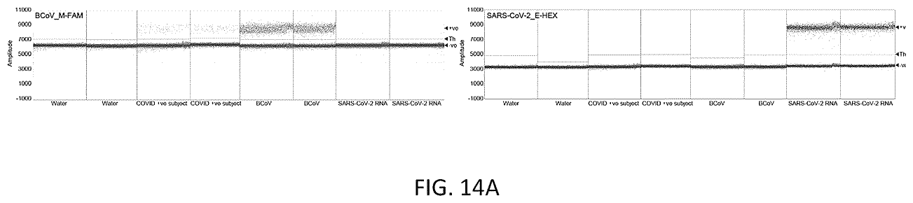

We found that in the nine stormwater samples, crAssphage ORF000024 had the highest median concentration of 4.65, with a standard deviation of 0.56 and IQR of 0.66 log₁₀ copies/L of stormwater, followed by the ToBRFV RdRP gene, with a median of 3.48, standard deviation of 0.97, and IQR of 1.24 log₁₀ copies/L of stormwater, the ToBRFV Mo gene, with a median of 3.34, standard deviation of 0.99, and IQR of 1.36 logo copies/L of stormwater, and finally the PMMoV CP gene, with a median of 3.02, standard deviation of 0.54, and IQR of 0.44 log₁₀ copies/L of stormwater (FIG. 13). Pairwise comparison of gene target concentrations across samples using the Wilcoxon signed-rank test revealed that differences in concentrations are not statistically significant and gene targets are similarly abundant. The concentration of gene targets in each of the samples is presented in FIG. 13. Notably, the ToBRFV Mo gene was detected in as many samples (6/9) as crAssphage ORF000024 (FIG. 6). This result suggests that using an RNA-based marker from ToBRFV to detect human stool contamination of stormwater may be as useful as using the DNA marker from crAssphage ORF000024.

We obtained concentrations of PMMoV and ToBRFV gene targets using templates that were diluted 1:10. Higher dilutions led to lower detection of these gene targets. Therefore, we believe that the results reported here are free of influence from RT-PCR inhibition.

Conclusions and limitations. In this study, we generated eight nearly complete genomes of ToBRFV from wastewater and stool from the Bay Area. We catalogued SNPs in all existing genomes, including in those that we assembled here, and noted variations in viral genomes isolated from the same individual over about 100 days. We then went on to identify two sets of primers and probes that can universally detect ToBRFV across the world.

Assays developed using these primer and probe sequences are sensitive and specific for human stool and wastewater, as they were present in a wide range of wastewaters and stool samples and not present in any tested animal stool aside from one sample from a cat and another from a pig. Like the established viral MST target PMMoV (8), the ToBRFV target is derived from the genome of a plant virus likely present in the human stool owing to dietary intake of diseased plants. Concentrations of ToBRFV Mo and RdRP gene targets were as high as or higher than those of the PMMoV CP gene in wastewater and stormwater known to contain sewage. The high concentrations of ToBRFV targets in wastewater, as well as in human stool samples, suggest that they may be useful as endogenous fecal-strength controls for wastewater-based epidemiology applications (23), as well as an endogenous positive extraction control during nucleic acid extractions in studies seeking to quantify rare infectious-disease targets (8) in human stool.

Notably, we took a number of actions in our analytical workflow to guard against the inhibition of RT-PCRs by substances that can coelute with the nucleic acid templates. First, we purified all nucleic acids using commercial kits that are known to remove such inhibitors. In the case of wastewater samples, we acquired templates from a previous study that additionally employed an inhibitor removal kit (21). Second, we used ddRT-PCR, which is less sensitive to inhibition than RT-qPCR (24, 25). Finally, we diluted the nucleic acid templates used in the ddRT-PCRs to mitigate the effect of inhibitors. In instances where the nucleic acid concentration was low, we report data from undiluted templates and used the diluted templates to assess the presence of inhibitors. Overall, we identified little evidence of inhibition.

There are several limitations to this work. First, the specificity of the ToBRFV Mo and RdRP gene targets was tested using just one representative sample each of various non-human animal stools. Additional work to test more animal stool samples would be helpful to further characterize the assays' specificity for human stool. Second, the sensitivities of the various assays were tested using human stool samples only from individuals residing in the Bay Area. It is possible that the distribution of the targets in individuals from other locations may differ from those studied here, and more work to document the ToBRFV prevalence and abundance in samples globally is encouraged. Third, it is possible that the extraction methods used to acquire nucleic acids from the various samples may have biases for the gene targets assayed in this study. Since ToBRFV and PMMoV both belong to the genus Tobamovirus, we believe that they are likely treated similarly by the extraction methods. However, in the stormwater samples, we compared gene targets from these viruses with those from crAssphage. crAssphage may react differently in the extraction process, and such variations are yet to be studied. Fourth, differences in the storage conditions of samples used in this study may have influenced our results (26). For instance, we have found that freezing and thawing samples can influence viral quantification, while differences in the duration of sample storage have a negligible effect (27). Notably, all frozen samples used in the current study underwent only one freeze-thaw cycle for this project. However, samples were stored for different durations, and this may have impacted our results in ways we cannot quantify. Biobanking of samples is a vital step in this research, and inherent variations in duration of storage are unavoidable. Fifth, we assayed wastewater solids sampled from around the United States, from New York to California, and they contained high concentrations of the ToBRFV targets. Further work using samples from around the world will be valuable to testing the generalizability of the assays. Notably, the presence of ToBRFV genomes from this study and others collected from many countries reassures us that ToBRFV is likely to be a universal global MST marker. Finally, as more ToBRFV genomes become available, it will be important to test whether the primers and probes developed herein continue to overlap conserved regions of the genomes.

### Materials and Methods

Assembly and analysis of ToBRFV genomes and design of hydrolysis probe RT-PCR assays. In order to design ToBRFV-specific primers and probes for hydrolysis-probe RT-PCR assays, all ToBRFV genomes available in February 2021 were obtained. These were supplemented with new genomes assembled from stool samples processed and sequenced in this study (Table 2).

In February 2021, all nearly complete genomes (n=70) of ToBRFV were downloaded from NCBI GenBank. In the same month, raw reads from the only publicly available wastewater metatranscriptomics data set (obtained from wastewater in the Bay Area, collected between May and July 2020; BioProject accession no. PRJNA661613) were also downloaded. Using these reads, five ToBRFV genomes were assembled as outlined below.

In addition to using existing sequencing data and genomes, RNA from three human stool samples obtained longitudinally from one individual were also sequenced; the first two samples were collected 10 days apart, and the third was collected 93 days after the second sample. The samples were obtained from an individual with laboratory-confirmed COVID-19 and were collected under an Institutional Review Board (IRB)-approved protocol (Stanford IRB protocol 55619). Total RNA was extracted from these samples, rRNA was depleted, and libraries were prepared and sequenced using NextSeq 550 as outlined in the supplemental material.

The following bioinformatic methods were used to assemble genomes from both the existing (from wastewater) and newly obtained (from stool) metatranscriptomics reads. Reads were trimmed with Trim Galore (version 0.4.0) using Cutadapt (version 1.8.1) (28) set to flags -q 30 and -illumina. SPAdes (version 3.14.1) set to -meta was used to assemble genomes de novo (28, 29). Contigs belonging to ToBRFV were classified using One Codex (30). Genes were annotated using Prodigal (version 2.6.3) set to -meta (31). If all genes were predicted on the negative strand of the contig, the entire contig was reverse complemented. The completeness of potential ToBRFV genomes was assessed using CheckV (version 1.0.1) (32), and genomes that were 0.90.0% complete were selected for subsequent analyses.

To assess strain diversity of ToBRFV in the longitudinal stool samples, RNA sequencing reads from stool samples were aligned to the ToBRFV reference genome (NCBI accession no. NC_028478) using Bowtie (version 2.4.2) (33). The resulting bam files were used as input into inStrain (version 1.0.0) (34) to calculate population-level average nucleotide identity (popANI) between genomes.

To assess abundance of ToBRFV relative to other viruses in the RNA sequencing (RNASeq) data, reads were classified against the Viral Kraken2 database (benlangmead.github.io/aws-indexes/k2) (35) using default parameters. Counts from the classification were used to calculate relative abundance of viral reads.

A multiple-sequence alignment of all nearly complete genomes of ToBRFV, including genomes downloaded from NCBI GenBank in February 2021 (70 genomes) and those we assembled from wastewater and stool (8 genomes), was performed using Geneious Alignment (Geneious Prime version 2021.0.3) (36) with default settings, global alignment with free end gaps, and cost similarity matrix set to 65.0%. SNPs were called from the multiple sequence alignment using SNP-Sites (version 2.5.1) (37). A phylogenetic tree was built using Geneious Tree Builder (version 2021.0.3) with default settings and a Tamura-Nei genetic distance model with the neighbor-joining method. Primers and probes were designed to be specific for ToBRFV using Geneious Primer (version 3 2.3.7) (38) based on the 78 genomes we had access to in February 2021 with near-default settings, requiring product size to be between 95 and 125 bp and primers to be based on the consensus with 100.0% identity across all ToBRFV genomes. Primers and probe sequences were screened for specificity, in silico, using NCBI BLAST.

New genomes available in November 2022. New ToBRFV genomes became available on public databases between the first phase of this study in February 2021 and the completion of this work in November 2022. Specifically, an additional 113 genomes were downloaded from NCBI, bringing the total to 183 (39) and 250 assembled ToBRFV genomes from a study of wastewater from Southern California (18) were downloaded (Table 2).

As Geneious alignment and tree building are computationally intensive, a phylogenetic tree of all 441 nearly complete genomes of ToBRFV was built using ViPTree (40) and visualized and color coded by region using Iroki (41). In addition, the applicability of the primers and probes designed in this study was tested in silico using NCBI BLAST.

Processing of animal stool samples for RNA quantification. One stool sample each was collected from (i) a single animal (cat, dog, horse, pig, and rabbit) raised as a pet, (ii) a group of cohabiting animals of a single kind (chicken, cow, goat, mouse, and sheep) from Deer Hollow Farms (California, USA), (iii) a group of cohoused ducks and geese at Deer Hollow Farms, and (iv) wild animals (bear and deer). Samples were collected in a sterile clinical stool collection container by individuals wearing gloves and using a spatula. Samples were transported at room temperature, aliquoted into cryovials, and stored at 280° C. within 12 h from collection. Samples were further processed within a month of storage and did not go through any freeze-thaw cycles prior to the current work.

A single, defined solid volume of sample of each animal stool was acquired using Integra Miltex biopsy punches with a plunger system (Thermo Fisher Scientific; catalog no. 12-460-410) and placed in independent microcentrifuge tubes. Five hundred microliters of RNAlater (Ambion; catalog no. AM7023M) was added, and samples were processed using a previously validated methodology (24) as follows. A stock BCoV vaccine was prepared by adding 3 mL of 1× phosphate-buffered saline (PBS; Fisher Scientific; catalog no. BP399-500) to one vial of lyophilized Zoetis Calf-Guard bovine rotavirus-coronavirus vaccine (catalog no. VLN 190/PCN 1931.20) to create an undiluted reagent as per the manufacturer's instructions. Ten microliters of this attenuated BCoV vaccine was added to every sample as an external control and vortexed for 15 min. BCoV is an RNA virus that was previously found to be a reliable positive control for RNA extraction from stool (24). Samples were processed immediately after addition of the BCoV control.

Collection and processing of human stool samples used for RNA quantification. Human stool samples were previously collected and biobanked in RNAlater solution as part of Stanford Institutional Review Board-approved protocols 8903 (Blood and Bone Marrow Grafting for Leukemia and Lymphoma), 11062 (Genome, Proteome and Tissue Microarray Studies in Childhood malignant and Non-Malignant Hematologic Disorders), and 48548 (Hematopoietic Recovery During Induction Chemotherapy in Pediatric Leukemia). From these biobanks, 194 and 28 samples collected from 125 adult and 4 pediatric participants, respectively, from November 2019 to October 2020 were used in this study. These samples had been stored for between 1 and 12 months depending on the date of collection and did not go through any freeze-thaw cycles prior to the current work. All samples were spiked with 10 mL of attenuated BCoV vaccine as a control and processed similarly to the animal stool samples.

RNA extraction from all stool samples used for RNA quantification. RNA was extracted from stool samples using the QIAamp viral RNA minikit (Qiagen; catalog no. 52906) as previously optimized (24). Briefly, the prepared stool samples were spun down at 10,000×g for 2 min to acquire 140 mL of clarified supernatant. RNA was extracted from this supernatant using the QIAamp viral RNA minikit (Qiagen; catalog no. 52906) as per the manufacturer's instructions. Finally, RNA was eluted in 100 mL of the elution buffer and stored in a 96-well plate at 280° C. for up to 12 months. Notably, in previous work on BCoV and SARS-CoV-2 RNA (24), we found that RNA extracted using this methodology was free of RT-PCR inhibitors.

Augmenting analysis of stool with metatranscriptomic data from healthy individuals. As described below, we assessed the prevalence and abundance of MST markers in stool acquired from participants with hematologic disorders. This presented an obstacle to the generalizability of our work. Therefore, we acquired metatranscriptomics data from stool samples from 10 healthy participants presented in a previous study (20). Though many human stool metatranscriptomic data sets exist, this was the most recent data set we had access to.

Collection and processing of wastewater samples used for RNA quantification. Settled solids were obtained from 15 wastewater treatment plants across the United States (Table S2). Solids were collected from the primary clarifier or settled from a 24-h composited influent sample using Imhof cones. Samples were collected in sterile containers and transported to the lab. Samples from the Bay Area were processed immediately, while other samples were stored at 280° C. until analysis (between 5 and 20 months). None of the samples stored at 280° C. underwent a freeze-thaw cycle prior to the current work.

Solids were dewatered using centrifugation, and then an aliquot of the dewatered solids was set aside for dry-weight analysis. Solids were then suspended in a buffer (approximately 75 mg/mL), homogenized, and centrifuged. This suspension of solids in buffer was found to alleviate inhibition of RT-PCR (21). An aliquot of the supernatant was processed for total nucleic acid extraction using Chemagic 360 (Perkin Elmer). Nucleic acid preparations from wastewater samples are known to contain PCR inhibitors that interfere with their accurate quantification using PCR-based methods. Therefore, inhibitors were removed using the OneStep PCR inhibitor removal kit (Zymo Research; catalog no. D6035), yielding nucleic acids in 50 mL of eluant. These methods have been published in detail (42), and step-by-step protocols are available on protocols.io (43, 44).

Source of RNA extracted from stormwater samples used for RNA quantification. RNA extracted from stormwater samples was derived from a previous study from our group (22). Briefly, nine stormwater samples from the Bay Area—one each from the Guadalupe River, Pilarcitos Creek, San Francisquito Creek, and San Pedro Creek, two from Stevens Creek, and three from Lobos Creek-collected between October 2018 and March 2019 were used to extract RNA (Table S3). Specifically, stormwater samples were collected in the winter of 2018-2019, and immediately upon collection, viruses were concentrated from 1 to 5.5 L of stormwater using electronegative filtration using 0.05 M MgCl₂. The filtration membranes were preserved in 250 ml of RNAlater (Qiagen; catalog no. 76104) for 5 min prior to storage at 280° C.

Nucleic acids were extracted into 100 mL of RNase-free water from the stored filtration membrane with a Qiagen DNA/RNA AllPrep PowerViral kit using a protocol including β-mercaptoethanol and bead beating and stored as aliquots in microcentrifuge tubes at 280° C. Extraction of nucleic acids was completed within 6 months of sample collection. Samples were thawed on ice prior to use in crAssphage assays. Separate additional frozen aliquots of extracted nucleic acids that had not undergone any freeze-thaw cycles were stored at 280° C. and used in the current work. Previous work suggested that RT-PCR inhibitors from the samples were not coextracted in this RNA extraction process (22). These extracts were used in the current study after 30 months of storage.

Quantification of viral RNA sequences by ddRT-PCR. The CP gene encoding the coat protein from PMMoV, the genes encoding the movement protein (Mo) and RNA-dependent RNA polymerase (RdRP) from ToBRFV, and the gene encoding the membrane (M) protein from BCoV were quantified using ddRT-PCR. We chose ddRT-PCR instead of RT-qPCR for nucleic acid detection and quantification because of its superior sensitivity and resistance to PCR inhibitors (24, 25).

Templates derived from non-human animal stool were assayed for the PMMoV and ToBRFV gene targets in their undiluted and 1:10 diluted formats. Templates derived from human stool were assayed for the PMMoV and ToBRFV gene targets in their undiluted format. However, 30 of these templates yielded ddRT-PCRs where all the droplets were positive. This is not ideal, because ddRT-PCRs rely on a Poisson distribution of the template across droplets to accurately quantify gene targets. Therefore, these templates were diluted 1:10,000 and reassayed for the relevant gene targets. Additionally, templates from eight samples were randomly chosen, diluted 1:10, and assayed for the PMMoV and ToBRFV gene targets to detect any inhibitors. Templates derived from wastewater samples were diluted 1:10,000 before assaying for PMMoV and ToBRFV gene targets. In cases where these gene targets were undetectable at this high dilution, we assayed templates at 1:100 and 1:10 dilutions and in an undiluted format. Templates derived from stormwater samples were assayed for the PMMoV and ToBRFV gene targets at three dilutions: 1:10,000, 1:1,000, and 1:10. Results reported are from the 1:10 dilution, since the gene targets were undetectable at higher dilutions.

Human participants in this study were enrolled and hospitalized during the first year of the COVID-19 pandemic. We tested their stools for genes encoding the envelope (E) and a nucleocapsid (N2) protein from the SARS-CoV-2 genome as previously described (24), in order to assess occurrence of COVID-19 during hospitalization at Stanford Hospital. However, we did not find any presence of COVID-19 RNA in these samples. Sequences of the newly designed primers and probes targeting ToBRFV Mo and RdRP genes are listed in Table 1. Previously published primers and probes targeting BCoV, PMMoV, and SARS-CoV-2 RNAs are listed in Table S4.

The droplet digital PCR application guide for QX200 machines (Bio-Rad) (45) and digital minimum information for publication of quantitative real-time PCR experiments (dMIQE) guidelines (46) inform this methodology. The experimental checklist recommended by dMIQE is available at the Stanford Digital Repository (purl.stanford.edu/nf771cs9443). A Biomek FX liquid handler (Beckman Coulter) was used to prepare the ddRT-PCR by adding 5.5 mL of eluted RNA to 5.5 mL supermix, 2.2 mL reverse transcriptase, 1.1 mL of 300 nM dithiothreitol (DTT), 1.1 mL of each of the 20× custom ddPCR assay primer-probe mixes (Bio-Rad; catalog no. 10031277), and 5.5 mL of nuclease-free water (Ambion; catalog no. AM9937, lot 2009117). The supermix, reverse transcriptase, and DTT were from the one-step ddRT-PCR Advanced kit for probes (Bio-Rad; catalog no. 1864021). A QX200 AutoDG droplet digital PCR system (Bio-Rad) was used to partition the samples into droplets of roughly 1 nL using the default settings, and the template was amplified using a Bio-Rad T100 thermocycler with the following thermocycling program: 50° C. for 60 min, 95° C. for 10 min, 40 cycles of 94° C. for 30 s and 55° C. for 1 min, followed by 1 cycle of 98° C. for 10 min and 4° C. for 30 min with a ramp speed of 1.6° C. per s at each step (47).

A multistep approach was adopted to calculate the raw RNA concentrations, as previously described (24). Every plate in the ddRT-PCR assays included appropriate positive and negative controls, including synthetic target genes (PMMoV CP gene, and ToBRFV Mo and RdRP genes) cloned in the pIDT vector, RNA extracted from reconstituted attenuated BCoV vaccine, water, and RNAlater. The signal threshold corresponding to every plate was manually set between the mean positive and negative amplitudes of these controls such that the number of detected copies in the negative controls was minimal and those from the relevant positive controls most closely matched the expected RNA concentration. Next, the difference between the mean negative amplitude and the threshold amplitude in the negative-control reactions was calculated and added to the mean negative amplitude for every sample on that plate. Applying this threshold yielded the raw RNA concentrations.

In order to derive the limit of blank (LoB) and limit of detection (LoD) of our assays to further process the raw RNA concentrations we adopted the following steps. (i) The LoB indicates the highest background RNA concentration registered from control samples that are confidently negative for the relevant gene targets. In order to determine the LoB, water, RNAlater, and synthetic genes discordant with the target gene (e.g., the ToBRFV Mo gene is a negative control in an assay of the ToBRFV RdRP gene) were assayed in duplicate. The highest RNA concentration measured in these LoB samples for each of the primer/probe sets was set as the relevant LoB. All samples in which we detected an RNA concentration equal to or less than the LoB were set to zero. (ii) The LoD is defined as the lowest concentration of RNA that can be reliably detected. To determine the LoD, duplicate serial dilution series of the synthetic target genes at 1, 2, 5, 10, 100, and 1,000 copies/mL of template were assayed for the corresponding target gene (FIG. 7). The synthetic target genes were acquired from Integrated DNA Technologies and cloned in their standard backbone, pIDTSmart. These plasmids were transformed into E. coli, isolated using the QIAprep Spin mini-prep kit (Qiagen; catalog no. 27104) and quantified using Qubit. The LoD for a primer/probe set was defined as the lowest concentration of the standard at which both replicates had a detectable RNA concentration. All viral RNA concentrations below the LoD were set to zero.

Finally, after these data processing and analysis steps, the samples were assigned a final viral RNA concentration in copies per microliter of template. “Eluate” refers to the 100 ml of sample acquired from the RNA extraction. Viral RNA concentrations from animal and human stool samples are expressed in copies per microliter of template, those from wastewater samples are in copies per gram of wastewater, and those from stormwater samples are in copies per liter of stormwater.

In the case of all non-human animal stool, wastewater and stormwater samples, RNA was quantified using singleplex reactions. For the human stool samples, which were limited in quantity, the detection of the BCoV M gene and PMMoV CP gene were multiplexed with the detection of the SARS-CoV-2 E and N2 genes using orthogonal fluorescent probes. After extensive optimization (outlined in the supplemental material), we paired the detection of the E gene (SARS-CoV-2) with that of the CP gene (PMMoV) and detection of the N2 gene (SARS-CoV-2) with that of the M gene (BCoV) in two independent reactions using the carboxyfluorescein (FAM) and hexachlorofluorescein (HEX) fluors, respectively.

Data analysis and generation of plots. Data were analyzed using RStudio (v 1.2.5042), using the packages cowplot (v 1.1.1), dplyr (v 1.0.8), eulerr (v 6.1.1), ggplot2 (v 3.3.6), and UpSetR (v 1.4.0).

Data availability. Newly generated genomes and raw sequencing reads from stool samples are available on NCBI's Sequence Read Archive (SRA) database under accession no. PRJNA917455. All other relevant data are included herein and available in the Stanford Digital Repository (purl.stanford.edu/nf771cs9443).

### REFERENCES

1. Whitman R L, Shively D A, Pawlik H, Nevers M B, Byappanahalli M N. 2003. Occurrence of Escherichia coli and enterococci in cladophora (Chlorophyta) in nearshore water and beach sand of Lake Michigan. Appl Environ Microbiol 69:4714-4719.
2. U.S. EPA. 2013. Recreational water quality criteria and methods.
3. U.S. EPA. 2015. Drinking water regulations.
4. Layton B A, Walters S P, Lam L H, Boehm A B. 2010. Enterococcus species distribution among human and animal hosts using multiplex PCR. J Appl Microbiol 109:539-547.
5. Imamura G J, Thompson R S, Boehm A B, Jay J A. 2011. Wrack promotes the persistence of fecal indicator bacteria in marine sands and seawater. FEMS Microbiol Ecol 77:40-49.
6. Yamahara K M, Walters S P, Boehm A B. 2009. Growth of enterococci in unaltered, unseeded beach sands subjected to tidal wetting. Appl Environ Microbiol 75:1517-1524.
7. Byappanahalli M N, Whitman R L, Shively D A, Sadowsky M J, Ishii S. 2006. Population structure, persistence, and seasonality of autochthonous Escherichia coli in temperate, coastal forest soil from a Great Lakes watershed. Environ Microbiol 8:504-513.
8. McClary-Gutierrez J S, Aanderud Z T, Al-Faliti M, Duvallet C, Gonzalez R, Guzman J, Holm R H, Jahne M A, Kantor R S, Katsivelis P, Kuhn K G, Langan L M, Mansfeldt C, Mclellan S L, Grijalva L M M, Murnane K S, Naughton C C, Packman A I, Paraskevopoulos S, Radniecki T S, Roman F A, Jr, Shrestha A, Stadler L B, Steele J A, Swalla B M, Vikesland P, Wartell B, Wilusz C J, Wong J C C, Boehm A B, Halden R U, Bibby K, Vela J D. 2021. Standardizing data reporting in the research community to enhance the utility of open data for SARS-CoV-2 wastewater surveillance. Environ Sci (Camb) 7:1545-1551.
9. Boehm A B, Van De Werfhorst L C, Griffith J F, Holden P A, Jay J A, Shanks O C, Wang D, Weisberg S B. 2013. Performance of forty-one microbial source tracking methods: a twenty-seven lab evaluation study. Water Res 47:6812-6828.
10. Shanks O C, White K, Kelty C A, Hayes S, Sivaganesan M, Jenkins M, Varma M, Haugland R A. 2010. Performance assessment PCR-based assays targeting bacteroidales genetic markers of bovine fecal pollution. Appl Environ Microbiol 76:1359-1366.
11. Green H C, Dick L K, Gilpin B, Samadpour M, Field K G. 2012. Genetic markers for rapid PCR-based identification of gull, Canada goose, duck, and chicken fecal contamination in water. Appl Environ Microbiol 78:503-510.
12. García-Aljaro C, Ballesté E, Muniesa M, Jofre J. 2017. Determination of crAssphage in water samples and applicability for tracking human faecal pollution. Microb Biotechnol 10:1775-1780.
13. Rosario K, Symonds E M, Sinigalliano C, Stewart J, Breitbart M. 2009. Pepper mild mottle virus as an indicator of fecal pollution. Appl Environ Microbiol 75:7261-7267.
14. Edwards R A, Vega A A, Norman H M, Ohaeri M, Levi K, Dinsdale E A, Cinek O, Aziz R K, McNair K, Barr J J, Bibby K, Brouns S J J, Cazares A, de Jonge P A, Desnues C, Díaz Muñoz S L, Fineran P C, Kurilshikov A, Lavigne R, Mazankova K, Mccarthy D T, Nobrega F L, Reyes Muñoz A, Tapia G, Trefault N, Tyakht A V, Vinuesa P, Wagemans J, Zhernakova A, Aarestrup F M, Ahmadov G, Alassaf A, Anton J, Asangba A, Billings E K, Cantu V A, Carlton J M, Cazares D, Cho G-S, Condeff T, Cortes P, Cranfield M, Cuevas D A, De la Iglesia R, Decewicz P, Doane M P, Dominy N J, Dziewit L, Elwasila B M, Eren A M, et al. 2019. Global phylogeography and ancient evolution of the widespread human gut virus crAssphage. Nat Microbiol 4:1727-1736.
15. Colson P, Richet H, Desnues C, Balique F, Moal V, Grob J-J, Berbis P, Lecoq H, Harlé J-R, Berland Y, Raoult D. 2010. Pepper mild mottle virus, a plant virus associated with specific immune responses, fever, abdominal pains, and pruritus in humans. PLoS One 5:e10041.
16. Zhang S, Griffiths J S, Marchand G, Bernards M A, Wang A. 2022. Tomato brown rugose fruit virus: an emerging and rapidly spreading plant RNA virus that threatens tomato production worldwide. Mol Plant Pathol 23:1262-1277.
17. Crits-Christoph A, Kantor R S, Olm M R, Whitney O N, Al-Shayeb B, Lou Y C, Flamholz A, Kennedy L C, Greenwald H, Hinkle A, Hetzel J, Spitzer S, Koble J, Tan A, Hyde F, Schroth G, Kuersten S, Banfield J F, Nelson K L. 2021. Genome sequencing of sewage detects regionally prevalent SARS-CoV-2 variants. mBio 12:e02703-20.
18. Rothman J A, Whiteson K L. 2022. Sequencing and variant detection of eight abundant plant-infecting tobamoviruses across Southern California wastewater. Microbiol Spectr 10:e03050-22.
19. Rothman J A, Loveless T B, Kapcia J, 3rd, Adams E D, Steele J A, Zimmer-Faust A G, Langlois K, Wanless D, Griffith M, Mao L, Chokry J, Griffith J F, Whiteson K L. 2021. RNA viromics of Southern California wastewater and detection of SARS-CoV-2 single-nucleotide variants. Appl Environ Microbiol 87:e01448-21.
20. Maghini D, Dvorak M, Dahlen A, Roos M, Kuersten S, Bhatt A S. 2023. Quantifying bias introduced by sample collection in relative and absolute microbiome measurements. Nat Biotechnol. doi.org/10.1038/s41587-023-01754-3.
21. Huisman J S, Scire J, Caduff L, Fernandez-Cassi X, Ganesanandamoorthy P, Kull A, Scheidegger A, Stachler E, Boehm A B, Hughes B, Knudson A, Topol A, Wigginton K R, Wolfe M K, Kohn T, Ort C, Stadler T, Julian T R. 2022. Wastewater-based estimation of the effective reproductive number of SARS-CoV-2. Environ Health Perspect 130:57011.
22. Graham K E, Anderson C E, Boehm A B. 2021. Viral pathogens in urban stormwater runoff: occurrence and removal via vegetated biochar-amended biofilters. Water Res 207:117829.
23. McClary-Gutierrez J S, Mattioli M C, Marcenac P, Silverman Al, Boehm A B, Bibby K, Balliet M, de Los Reyes F L, Gerrity D, Griffith J F, Holden P A, Katehis D, Kester G, LaCross N, Lipp E K, Meiman J, Noble R T, Brossard D, Mclellan S L. 2021. SARS-CoV-2 wastewater surveillance for public health action. Emerg Infect Dis 27:1-8.
24. Natarajan A, Han A, Zlitni S, Brooks E F, Vance S E, Wolfe M, Singh U, Jagannathan P, Pinsky B A, Boehm A, Bhatt A S. 2021. Standardized preservation, extraction and quantification techniques for detection of fecal SARS-CoV-2 RNA. Nat Commun 12:5753.
25. Kuypers J, Jerome K R. 2017. Applications of digital PCR for clinical microbiology. J Clin Microbiol 55:1621-1628.
26. Franzosa E A, Morgan X C, Segata N, Waldron L, Reyes J, Earl A M, Giannoukos G, Boylan M R, Ciulla D, Gevers D, Izard J, Garrett W S, Chan A T, Huttenhower C. 2014. Relating the metatranscriptome and metagenome of the human gut. Proc Natl Acad Sci USA 111:E2329-E2338.
27. Simpson A, Topol A, White B J, Wolfe M K, Wigginton K R, Boehm A B. 2021. Effect of storage conditions on SARS-CoV-2 RNA quantification in wastewater solids. Peer J 9:e11933.
28. Martin M. 2011. Cutadapt removes adapter sequences from high-throughput sequencing reads. EMBnet J 17:10.
29. Nurk S, Meleshko D, Korobeynikov A, Pevzner P A. 2017. metaSPAdes: a new versatile metagenomic assembler. Genome Res 27:824-834.
30. Minot S S, Krumm N, Greenfield N B. 2015. One Codex: a sensitive and accurate data platform for genomic microbial identification. bioRxiv. doi.org/10.1101/027607.
31. Hyatt D, Chen G-L, Locascio P F, Land M L, Larimer F W, Hauser L J. 2010. Prodigal: prokaryotic gene recognition and translation initiation site identification. BMC Bioinformatics 11:119.
32. Nayfach S, Camargo A P, Schulz F, Eloe-Fadrosh E, Roux S, Kyrpides N C. 2021. CheckV assesses the quality and completeness of metagenome-assembled viral genomes. Nat Biotechnol 39:578-585.
33. Langmead B, Salzberg S L. 2012. Fast gapped-read alignment with Bowtie 2. Nat Methods 9:357-359.
34. Olm M R, Crits-Christoph A, Bouma-Gregson K, Firek B A, Morowitz M J, Banfield J F. 2021. inStrain profiles population microdiversity from metagenomic data and sensitively detects shared microbial strains. Nat Biotechnol 39:727-736.
35. Wood D E, Lu J, Langmead B. 2019. Improved metagenomic analysis with Kraken 2. Genome Biol 20:257.
36. Geneious. 2019. Geneious. geneious.com.
37. Page A J, Taylor B, Delaney A J, Soares J, Seemann T, Keane J A, Harris S R. 2016. SNP-sites: rapid efficient extraction of SNPs from multi-FASTA alignments. Microb Genom 2:e000056.
38. Rozen S, Skaletsky H. 2000. Primer3 on the WWW for general users and for biologist programmers. Methods Mol Biol 132:365-386.
39. Sayers E W, Bolton E E, Brister J R, Canese K, Chan J, Comeau D C, Farrell C M, Feldgarden M, Fine A M, Funk K, Hatcher E, Kannan S, Kelly C, Kim S, Klimke W, Landrum M J, Lathrop S, Lu Z, Madden T L, Malheiro A, Marchler-Bauer A, Murphy T D, Phan L, Pujar S, Rangwala S H, Schneider V A, Tse T, Wang J, Ye J, Trawick B W, Pruitt K D, Sherry S T. 2023. Database resources of the National Center for Biotechnology Information in 2023. Nucleic Acids Res 51:D29-D38.
40. Nishimura Y, Yoshida T, Kuronishi M, Uehara H, Ogata H, Goto S. 2017. ViPTree: the viral proteomic tree server. Bioinformatics 33:2379-2380.
41. Moore R M, Harrison A O, McAllister S M, Polson S W, Wommack K E. 2020. Iroki: automatic customization and visualization of phylogenetic trees. Peer J 8:e8584.
42. Wolfe M K, Topol A, Knudson A, Simpson A, White B, Vugia D J, Yu A T, Li L, Balliet M, Stoddard P, Han G S, Wigginton K R, Boehm A B. 2021. High-frequency, high-throughput quantification of SARS-CoV-2 RNA in wastewater settled solids at eight publicly owned treatment works in Northern California shows strong association with COVID-19 incidence. mSystems 6:e00829-21. doi.org/10.1128/mSystems.00829-21.
43. Topol A, Wolfe M, White B, Wigginton K, Boehm A B. 2021. High throughput pre-analytical processing of wastewater settled solids for SARS-CoV-2 RNA analyses. protocols.io/view/high-throughput-pre-analytical-processing-of-waste-kxygxpod4l8j/v2.
44. Topol A, Wolfe M, Wigginton K, White B, Boehm A. 2021. High throughput RNA extraction and PCR inhibitor removal of settled solids for wastewater surveillance of SARS-CoV-2 RNA V.2. protocols.io/view/high-throughput-rna-extraction-and-pcr-inhibitor-r-81wgb72bovpk/v2.
45. Bio-Rad. 2023. Droplet digital PCR applications guide (6407 ver B). Bio-Rad, Hercules, C A.
46. Huggett J F, dMIQE Group. 2020. The digital MIQE guidelines update: minimum information for publication of quantitative digital PCR experiments for 2020. Clin Chem 66:1012-1029.
47. Loeb S. 2020. One-step R T-ddPCR for detection of SARS-CoV-2, bovine coronavirus, and PMMoV RNA in RNA derived from wastewater or primary settled solids. doi.org/10.17504/protocols.io.bi6vkhe6.

TABLE 1

| | | | |
|---|---|---|---|
| | | | Amplicon |
| | | | length (bp) or |
| Primer or probe | Description | Sequence (59 to 39) | modificationsª |

Primers

| | | | |
|---|---|---|---|
| ToBRFV_Mo_F | ToBRFV Mo gene; | TCA GTG TCT GTT TGG TCG ATA A | 105 |
| | forward primer | (SEQ ID NO: 1) | |
| ToBRFV_Mo_R | ToBRFV Mo gene; | GGA ACG ACT TTG AAC TGA AAC C | |
| | reverse primer | (SEQ ID NO: 2) | |
| ToBRFV_RdRP_F | ToBRFV RdRP | AGC CAC AAG AGA TAA TGT TCG | 103 |
| | gene; forward | TA (SEQ ID NO: 4) | |
| | primer | | |
| ToBRFV_RdRP_R | ToBRFV RdRP | ACA TCA GAC CTT CGT CGA TAA | |
| | gene; reverse | AT (SEQ ID NO: 5) | |
| | primer | | |

Probes

| | | | |
|---|---|---|---|
| ToBRFV_Mo_P | ToBRFV Mo gene; | AGA GCG GAC GAG GCA ACT CTT | FAM/ZEN/IBHQ |
| | probe | G (SEQ ID NO: 3) | |
| ToBRFV_RdRP_P | ToBRFV RdRP | ACG GTA AAG GAA CAC GCT GTC | FAM/ZEN/IBHQ |
| | gene; probe | AGT (SEQ ID NO: 7) | |

^{a}FAM, 6-carboxyfluorescein; ZEN, proprietary to IDT; IBHQ, 3′-Iowa Black Fluorescent Quencher.

TABLE 2

| | | No. of ToBRFV genomes available in: | | Reference or source | |
|---|---|---|---|---|---|
| Sample | Sample | February | November | Sequence | Assembled |
| type | source | 2021 | 2022 | data | genomes |
| Stool | Bay Area, | 3 | 0 | This study | This study |
| | CA, USA | | | | |
| Tomatoes | Global | 70 | 183 | NA | ncbi.nlm.nih.gov/nuccore |
| Wastewater | Southern | 0 | 250 | NA | 18 |
| | CA, USA | | | | |
| Wastewater | Bay Area, | 5 | 0 | NCBI BioProject | This study |
| | CA, USA | | | (PRJNA661613) | |

TABLE S1
Demographic distribution of participants
who provided stool for ddRT-PCR

| | | Adult | | Pediatric |
|---|---|---|---|---|
| Characteristic | | (N = 125) | | (N = 4) |
| Median age (range), years | | 59.5 | (19-82) | 6 (3-16) |
| Age group - no. (%) | 10-20 | 1 | | Not reported** |
| | 20-30 | 12 | | |
| | 30-40 | 17 | | |
| | 40-50 | 11 | | |
| | 50-60 | 25 | | |
| | 60-70 | 45 | | |
| | 70-80 | 12 | | |
| | 80-90 | 1 | | |
| | Unknown* | 1 | | |
| Sex - no. (%) | Male | 79 | | Not reported** |
| | Female | 45 | | |
| | Unknown* | 1 | | |
| Race - no. (%) | White | 77 | (61.6%) | Not reported** |
| | Asian | 13 | (10.4%) | |
| | Black | 6 | (4.80%) | |
| | Unknown | 25 | (20.0%) | |
| | Not reported** | 8 | (6.40%) | |
| Ethnicity; | No | 105 | (84.0%) | Not reported** |
| Hispanic or | Yes | 19 | (15.2%) | |
| Latinx - no. (%) | Unknown | 1 | (0.800%) | |
| | Not reported** | 4 | (3.20%) | |

*Unknown refers to data that was not provided by participants.
**Not reported refers to data that is aggregated in order to avoid information that can be used to identify participants.

TABLE S2
Information on wastewater samples.

| | | | | Date of collection |
|---|---|---|---|---|
| Sample ID | Sample code | City | State | (YYYY-MM-DD) |
| Akron | OH-Akn | Akron | OH | 2020-06-10 |
| Boston | MA-Bos | Boston | MA | 2020-06-01 |
| Davis | CA-Dav | Davis | CA | 2021-07-17 |
| Gilroy | CA-Gil | Giloy | CA | 2021-07-17 |
| Hyperion | CA-LA | Los Angeles | CA | 2020-11-17 |
| New York | NY-NYC | New York City | NY | 2020-05-06 |
| North City | CA-SD | San Diego | CA | 2020-11-13 |
| Oceanside | CA-SF | San Francisco | CA | 2021-07-17 |
| Palo Alto | CA-PA | Palo Alto | CA | 2021-07-16 |
| Sacramento | CA-Sac | Sacramento | CA | 2021-07-17 |
| San Jose | CA-SJ | San Jose | CA | 2021-07-16 |
| Silicon | CA-SM | San Mateo | CA | 2021-07-17 |
| Valley | | | | |
| Sunnyvale | CA-Sun | Sunnyvale | CA | 2021-07-17 |
| UC Davis | CA-UCD | Davis | CA | 2021-07-08 |
| Wisconsin | WI-Mil | Milwaukee | WI | 2020-09-01 |

TABLE S3
Information on stormwater samples from California

| | | Date of collection |
|---|---|---|
| Sample ID | Location | (YYYY-MM-DD) |
| 24 | Guadalupe River | 2018-12-17 |
| 9 | Lobos Creek | 2019-02-21 |
| 15 | Lobos Creek | 2018-10-29 |
| 35 | Lobos Creek | 2019-01-17 |
| 45 | Pilarcitos Creek | 2018-10-10 |
| 27 | San Francisquito Creek | 2018-12-17 |
| 47 | San Pedro Creek | 2018-11-27 |
| 12 | Stevens Creek | 2018-10-10 |
| 34 | Stevens Creek | 2019-01-17 |

TABLE S4
Sequences of oligonucleotides used as primers and probes

| Primer | Description | Sequence (5 to 3′) | Ref |
|---|---|---|---|
| PMMOV_CP_F | PMMoV CP gene; | GAG TGG TTT GAC CTT | (2) |
| | forward primer | AAC GTT TGA (SEQ ID NO: 9) | |
| PMMoV_CP_R | PMMoV CP gene; | TTG TCG GTT GCA ATG | (2) |
| | reverse primer | CAA GT (SEQ ID NO: 10) | |
| BCOV_M_F | BCoV M gene; forward | CTG GAA GTT GGT GGA | (3) |
| | primer | GTT (SEQ ID NO: 11) | |
| BCOV_M_R | BCoV M gene; reverse | ATT ATC GGC CTA ACA TAC | (3) |
| | primer | ATC (SEQ ID NO: 12) | |
| SARS-COV-2_E_F | E gene forward primer | ACA GGT ACG TTA ATA GTT | (4) |
| | | AAT AGC GT (SEQ ID NO: 13) | |
| SARS-COV-2_E_R | E gene reverse primer | ATA TTG CAG CAG TAC | (4) |
| | | GCA CAC A (SEQ ID NO: 14) | |
| 2019-nCOV_N2-F | N2 gene forward primer | TTA CAA ACA TTG GCC | (5) |
| | | GCA AA (SEQ ID NO: 15) | |
| 2019-nCOV_N2-R | N2 gene reverse primer | GCG CGA CAT TCC GAA | (5) |
| | | GAA (SEQ ID NO: 16) | |
| PMMoV_CP_P | PMMoV CP gene; | CCT ACC GAA GCA AAT G | (2) |
| | probe | (SEQ ID NO: 17) | |
| BCOV_M_P | BCoV M gene; probe | CCT TCA TAT CTA TAC ACA | (3) |
| | | TCA AGT TGT T | |
| | | (SEQ ID NO: 18) | |
| SARS-COV-2_E_Prb-FAM | E gene probe | ACA CTA GCC ATC CTT ACT | (4) |
| | | GCG CTT CG | |
| | | (SEQ ID NO: 19) | |
| 2019-nCOV_N2-P | N2 gene probe | ACA ATT TGC CCC CAG | (5) |
| | | CGC TTC AG | |
| | | (SEQ ID NO: 20) | |

TABLE S5
Assessment of newly assembled ToBRFV genomes

| | | Estimated | |
|---|---|---|---|
| | Length | completeness | Sample |
| Sample ID | (bps) | (%) | type |
| 20200528WW - Berkeley | 6335 | 99.2 | Wastewater |
| 20200609WW - Berkeley | 6301 | 98.7 | sample |
| 20200519WW - Oakland | 5976 | 93.6 | |
| 20200528WW - Oakland | 6341 | 99.3 | |
| 20200609WW - Oakland | 6350 | 99.4 | |
| 2020_day1_St - | 6380 | 99.9 | Stool |
| Stanford | | | sample |
| 2020_day10_St - | 6385 | 100 | |
| Stanford | | | |
| 2020_day93_St - | 6386 | 100 | |
| Stanford | | | |

TABLE S6
Summary of template concentrations from all tested multiplexed reactions

| | | | | | ATCC | |
|---|---|---|---|---|---|---|
| | | | RNA | Attenuated | SARS- | |
| | | | from | BCoV | CoV- | |
| | | | COVID + ve | vaccine(1:100 | 2(10\hat{ }3 | |
| | | Water | participant | dilution) | copies/ | |
| Channel | Target | Concentration (copies/μL of template) | | | μL) | Notes based on FIG. 9 |
| 1 (FAM) | BCoV_M | 0.00 | 73.77 | 1840.66 | 0.00 | 1d plot of raw ddRT-PCR |
| | gene | | | | | amplitude reveals clean |
| 2 (HEX) | SARS- | 0.00 | 0.30 | 0.00 | 2406.84 | separation of signal from |
| | CoV-2_E | | | | | background |
| | gene | | | | | |
| 1 (FAM) | BCoV_M | 0.00 | 76.61 | 1910.56 | 0.14 | 1d plot of raw ddRT-PCR |
| | gene | | | | | amplitude reveals clean |
| 2 (HEX) | SARS- | 0.00 | 1.30 | 0.14 | 4373.69 | separation of signal from |
| | CoV- | | | | | background |
| | 2_N2 | | | | | |
| | gene | | | | | |
| 1 (FAM) | BCoV_M | 0.92 | 14189.80 | 2007.72 | NA | PMMoV_CP-HEX failed |
| | gene | | | | | |
| 2 (HEX) | PMMoV_CP | 0.00 | 0.00 | 0.00 | NA | |
| | gene | | | | | |
| 1 (FAM) | SARS- | 0.00 | 0.39 | NA | 2445.72 | 1d plot of raw ddRT-PCR |
| | CoV-2_E | | | | | amplitude reveals bleed through |
| | gene | | | | | of signal from the FAM to the |
| 2 (HEX) | PMMoV_CP | 2.02 | 171.71 | NA | 1.60 | HEX channel |
| | gene | | | | | |
| 1 (FAM) | SARS- | 0.00 | 0.82 | NA | 4055.90 | 1d plot of raw ddRT-PCR |
| | CoV- | | | | | amplitude reveals bleed through |
| | 2_N1 | | | | | of signal from the FAM to the |
| | gene | | | | | HEX channel |
| 2 (HEX) | SARS- | 0.15 | 0.41 | NA | 2454.43 | |
| | CoV-2_E | | | | | |
| | gene | | | | | |
| 1 (FAM) | SARS- | 2.89 | 2.32 | NA | 49.24 | 1d plot of raw ddRT-PCR |
| | CoV- | | | | | amplitude reveals that the FAM |
| | 2_N1 | | | | | channel is noisy and leading to |
| | gene | | | | | false positive RNa |
| 2 (HEX) | SARS- | 0.00 | 0.58 | NA | 97.56 | concentration in water |
| | CoV- | | | | | |
| | 2_N2 | | | | | |
| | gene | | | | | |
| 1 (FAM) | SARS- | 0.00 | 0.88 | NA | 4401.05 | 1d plot of raw ddRT-PCR |
| | CoV- | | | | | amplitude reveals bleed through |
| | 2_N1 | | | | | of signal from the FAM to the |
| | gene | | | | | HEX channel |
| 2 (HEX) | PMMoV_CP | 1.33 | 169.53 | NA | 2.69 | |
| | gene | | | | | |
| 1 (FAM) | SARS- | 0.00 | 0.32 | NA | 3620.74 | 1d plot of raw ddRT-PCR |
| | CoV- | | | | | amplitude reveals bleed through |
| | 2_N2 | | | | | of signal from the FAM to the |
| | gene | | | | | HEX channel |
| 2 (HEX) | PMMoV_CP | 0.69 | 103.21 | NA | 1.61 | |
| | gene | | | | | |
| 1 (FAM) | PMMoV_CP | 0.32 | 169.90 | NA | 0.16 | 1d plot of raw ddRT-PCR |
| | gene | | | | | amplitude reveals clean |
| 2 (HEX) | SARS- | 0.00 | 0.38 | NA | 2536.65 | separation of signal from |
| | CoV-2_E | | | | | background |
| | gene | | | | | |
| 1 (FAM) | PMMoV_CP | 3.30 | 1875.39 | NA | 1.89 | 1d plot of raw ddRT-PCR |
| | gene | | | | | amplitude reveals clean |
| 2 (HEX) | SARS- | 0.00 | 9.10 | NA | 48239.72 | separation of signal from |
| | CoV- | | | | | background |
| | 2_N2 | | | | | |
| | gene | | | | | |
| 1 (FAM) | SARS- | 0.00 | 0.00 | NA | 490.29 | 1d plot of raw ddRT-PCR |
| | CoV- | | | | | amplitude reveals that the HEX |
| | 2_RdRP | | | | | channel is noisy and leading to |
| | gene | | | | | false positive RNA |
| 2 (HEX) | PMMoV_CP | 0.97 | 105.02 | NA | 1.56 | concentration in water |
| | gene | | | | | |

### Example 2

Sequencing of Total RNA from Three Stool Samples.

RNA extraction was carried out as follows. 300-400 mg of each stool sample was added to 2 mL microfuge tubes containing 4 mm zirconium beads. To each sample, 250 μl of Tris-EDTA buffer (pH 7.4), 40 μL of lysozyme (Sigma-Aldrich; Catalog #L3790) at 10 mg/ml, 10 UL Qiagen lytic enzyme solution (Catalog #158928), and 10 μL metapolyzyme (Sigma-Aldrich; Catalog #MAC4L) at 10 mg/ml were added. Samples were then mixed by vortexing to disrupt the stool matrix, and incubated in a shaker incubator at 37° C. at 150 rpm for 15 minutes. Samples were treated with 20 μL of Proteinase K per sample (Qiagen; Catalog #19157) and incubated for another 15 minutes at 37° C. at 150 rpm. To enable mechanical lysis, 1 mL of RLT buffer (Qiagen; Catalog #79216) with 10 μL of 2-mercaptoethanol was added to each sample, followed by bead beating for 3 minutes and recovery on ice. Debris was removed by centrifugation at 4° C. at 21,000 g for 3 minutes. The supernatants were transferred into 5 mL tubes and an equal volume of acidic Phenol/Chloroform (pH 4.5; Invitrogen; Catalog #AM9722) was added to each sample. After vortexing for 3 minutes, the samples were spun down at 12,000 g at 4° C. for 10 minutes. The supernatants were transferred to new 5 mL tubes. To purify the RNA from stool-derived contaminants, the supernatants were further purified using the RNA Clean & Concentrator-5 kit (Zymo Research; Catalog #R1013). To each sample, 1 volume of ethanol (95-100%) was mixed with the supernatant from the previous step. The samples were transferred to the Zymo-Spin IC Columns in collection tubes and centrifuged. The rest of the purification protocol proceeded according to the kit specifications. The RNA samples were eluted off the columns by adding 30 μL of nuclease-free water directly to the column matrix followed by incubation for 15 minutes at room temperature then centrifugation into collection microfuge tubes. All samples were stored at −80° C. for up to 14 days until they were processed for RNA sequencing. Total RNA extracted from stool samples was subjected to rRNA depletion using a pre-commercial version of the RiboZero Plus Microbiome kit (Illumina). Following depletion, the samples were converted into libraries using the Illumina RNA Prep for Enrichment kit. To obtain shotgun metatranscriptomic information from the rRNA-depleted samples, the pre-enriched (Total RNAseq) libraries were sequenced on a NextSeq 550.

### Example 3

Identifying Primers/Probes that are Compatible in Multiplexed ddRT-PCR Assays

The QX200 ddPCR droplet reader enables the simultaneous detection or multiplexing of nucleic acids across two fluorescence channels where—channel 1 measures wavelength corresponding carboxyfluorescein (FAM) and channel 2, hexachlorofluorescein (HEX). Since we planned to assay for four target genes, two corresponding to the SARS-CoV-2 genomic RNA, and one each for the PMMoV and BCoV genomic RNA, we sought to maximize our throughput and save RNA extracted from precious clinical samples by multiplexing our reactions. However, this required us to evaluate the performance of primer/probe combinations targeting relevant genes. Notably, we want to pick combinations of primer/probes bearing orthogonal fluorophores such that they retain high signal-to-noise separation in their respective channels of detection while also avoiding interference of signal across channels. This is evaluated based on three observations in the 1-D amplitude plots from ddRT-PCR data:

Rain: In an ideal ddRT-PCR reaction, the amplitude of the droplets that are positive will cluster distinctly at a higher value than the amplitude of the droplets that are negative. However, when the reaction is not efficient, there ends up being a number of droplets that have an intermediate amplitude between the positive and negative values. These are referred to as “rain”. We want to pick primer/probes that have none to minimal rain.

Separation in amplitude between signal and noise: As previously mentioned, in ddRT-PCR, droplets that bear a positive signal will have a higher mean amplitude compared to the negative droplets. We want to pick primer/probes that had maximal differences between the mean positive and negative amplitude. This allows us to comfortably set the threshold between the positive and negative amplitudes.

Signal bleed through across channels: Theoretically, the FAM and HEX fluorescence signals are meant to be orthogonal, meaning a positive signal in one channel should not affect a read-out in the other. This is crucial to accurately quantify two different gene targets in a single reaction. However, some primer/probes lead to bleeding through of signal across channels. This means that where we should see droplets form clusters around two amplitudes for the positives and negatives respectively, we instead witness a third cluster representing signal from the orthogonal channel. The presence of bleed-through in the signal will interfere with the accurate quantification of target genes.

The United States Centers for Disease Control and Prevention (CDC) (6) and the German Centre for Infection Research (DZIF) (4) suggest four gene targets for PCR-based detection of SARS-CoV-2. These are the genes encoding the Envelope protein (E), Nucleocapsid proteins (N1, N2), and RNA-dependent RNA polymerase protein (RdRP). Previous literature has optimized primer/probes targeting the gene for the coat protein (CP) in PMMoV (2) and transmembrane protein (M) in BcoV (3). In a bid to find the best combination of primer/probes, we acquired oligonucleotides targeting the E, N1, N2, RdRP, CP and M genes tagged with FAM, and the E, N2 and CP genes tagged with HEX (Table S4). The repertoire of HEX labeled primer/probes tested herein was limited by the availability of reagents.

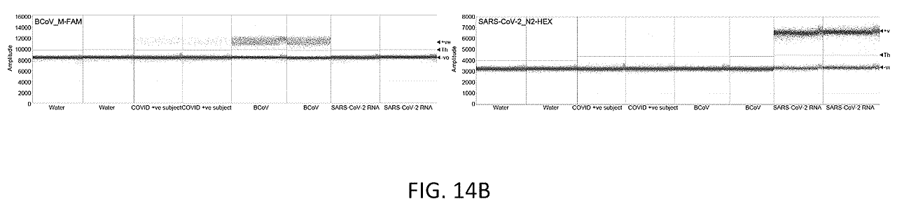

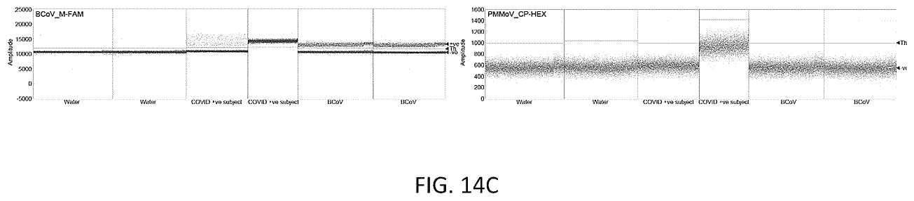

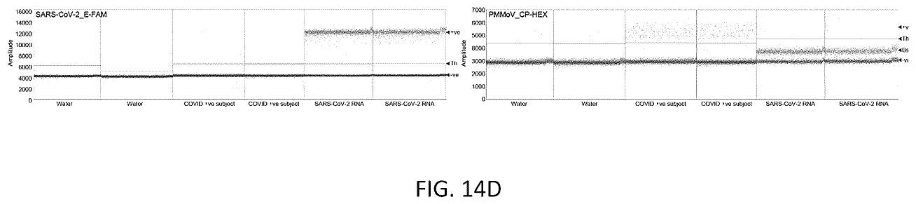

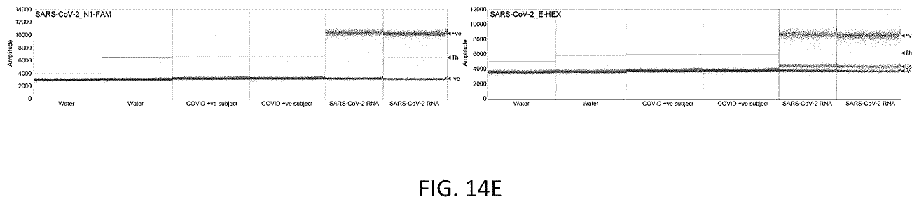

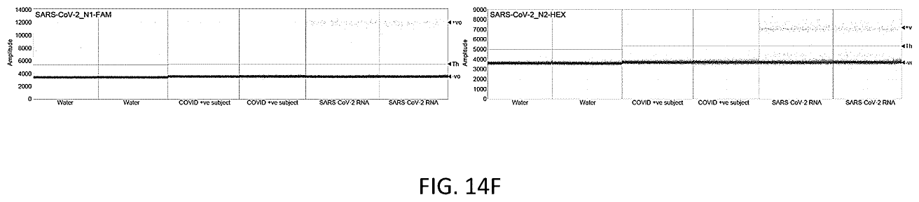

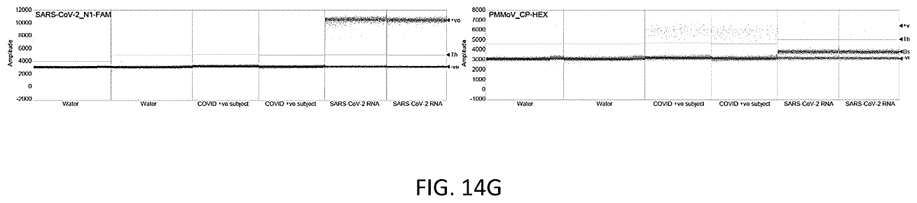

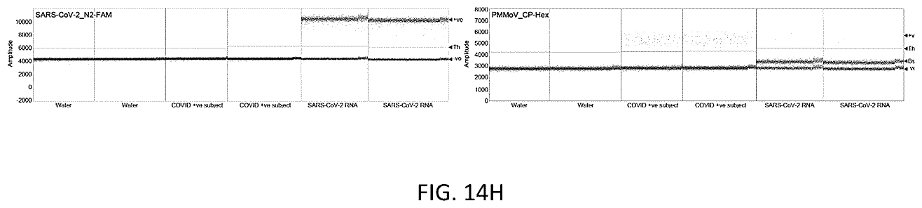

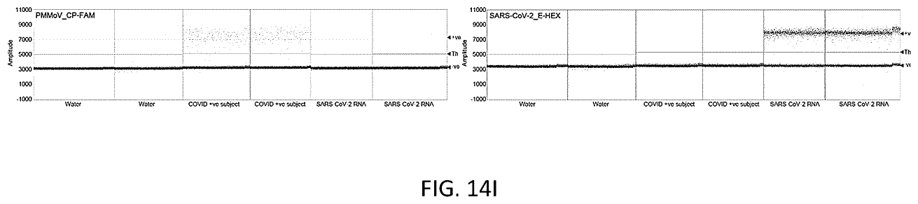

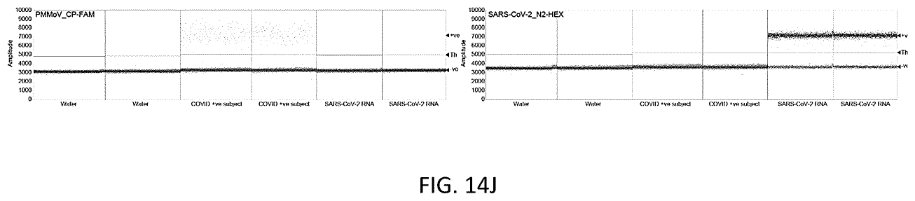

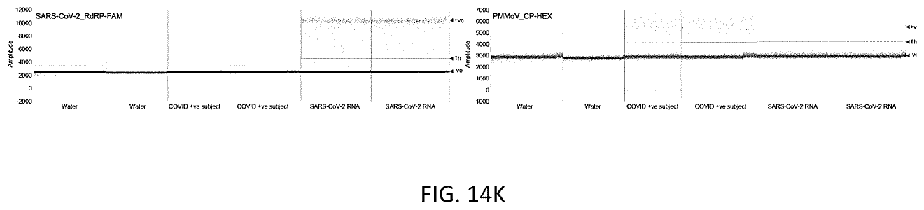

The raw 1-D amplitudes from these 11 combinations of primers and probes were analyzed to identify multiplexed reactions that did not feature rain and signal bleed through across channels (FIG. 14) and presented the best separation of signal from noise using appropriate control samples. Water is used as a universal no template control and synthetic SARS-CoV-2 viral RNA from ATCC is used as a positive control for SARS-CoV-2 target genes. Viral RNA extracted from 1) stool from a COVID-19 +ve participant who was admitted to the ICU is used as a positive control for SARS-CoV-2 and PMMoV viral RNAs, and 2) attenuated BCoV vaccine is used as a positive control for BCoV. These samples also serve as negative controls for the complementary primers and probes. Through this analysis, we identified that multiplexing the detection of the SARS-CoV-2 E gene and the PMMoV CP gene, and the SARS-CoV-2 N2 gene and the BCoV M gene in two independent multiplexed reactions using the FAM and HEX fluors respectively performed the best (FIG. 14, Table S6).

### REFERENCES

1. Graham K E, Anderson C E, Boehm A B. 2021. Viral pathogens in urban stormwater runoff: Occurrence and removal via vegetated biochar-amended biofilters. Water Res 207:117829.
2. Haramoto E, Kitajima M, Kishida N, Konno Y, Katayama H, Asami M, Akiba M. 2013. Occurrence of pepper mild mottle virus in drinking water sources in Japan. Appl Environ Microbiol 79:7413-7418.
3. Decaro N, Elia G, Campolo M, Desario C, Mari V, Radogna A, Colaianni M L, Cirone F, Tempesta M, Buonavoglia C. 2008. Detection of bovine coronavirus using a TaqMan-based real-time RT-PCR assay. J Virol Methods 151:167-171.
4. Corman V M, Landt O, Kaiser M, Molenkamp R, Meijer A, Chu D K, Bleicker T, Brünink S, Schneider J, Schmidt M L, Mulders D G, Haagmans B L, van der Veer B, van den Brink S, Wijsman L, Goderski G, Romette J-L, Ellis J, Zambon M, Peiris M, Goossens H, Reusken C, Koopmans M P, Drosten C. 2020. Detection of 2019 novel coronavirus (2019-nCoV) by real-time RT-PCR. Euro Surveill 25.
5. 2020. CDC 2019-Novel Coronavirus (2019-nCoV) Real-Time RT-PCR Diagnostic Panel. CDC-006-00019, Revision: 06. Division of Viral Diseases, Centers for Disease Control and Prevention.
6. Lu X, Wang L, Sakthivel S K, Whitaker B, Murray J, Kamili S, Lynch B, Malapati L, Burke S A, Harcourt J, Tamin A, Thornburg N J, Villanueva J M, Lindstrom S. 2020. US CDC Real-Time Reverse Transcription PCR Panel for Detection of Severe Acute Respiratory Syndrome Coronavirus 2. Emerg Infect Dis 26.

What is claimed is:

1. A method for selectively detecting tomato brown rugose fruit virus (ToBRFV) in a sample suspected of having human fecal contamination, the method comprising:
isolating nucleic acids from the sample suspected of having human fecal contamination, wherein if ToBRFV RNA is present, said nucleic acids comprise a target sequence;
amplifying the nucleic acids using a set of primers capable of selectively amplifying at least a portion of the ToBRFV RNA, wherein the ToBRFV RNA comprise the target sequence; and
detecting the presence of the amplified nucleic acids using a detectably labeled oligonucleotide probe sufficiently complementary to and capable of hybridizing with the ToBRFV RNA or amplicon thereof, if present, as an indication of the presence or absence of the ToBRFV in the sample, wherein said primers and said probe are capable of selectively hybridizing to the target sequence from the ToBRFV, wherein said detecting the presence of the ToBRFV in the sample indicates that human feces is present in the sample.

2. The method of claim 1, wherein the set of primers is capable of selectively amplifying at least a portion of a movement protein (Mo) encoding gene or at least a portion of an RNA dependent RNA polymerase (RdRP) encoding gene of the ToBRFV.

3. The method of claim 2, wherein the set of primers comprises:
(a) a forward primer comprising or consisting of the sequence of SEQ ID NO:1 and a reverse primer comprising or consisting of the sequence of SEQ ID NO:2;
(b) a forward primer comprising or consisting of the sequence of SEQ ID NO:4 and a reverse primer comprising or consisting of the sequence of SEQ ID NO:5;
(c) a forward primer and a reverse primer each comprising at least 10 contiguous nucleotides from the corresponding nucleotide sequences of the forward primer and reverse primer of a primer set selected from the group consisting of (a) and (b);
(d) a forward primer and a reverse primer comprising at least one nucleotide sequence that differs from the corresponding nucleotide sequence of the forward primer or reverse primer of a primer set selected from the group consisting of (a)-(c) in that the primer has up to three nucleotide changes compared to the corresponding sequence, wherein the primer is capable of hybridizing to and amplifying the ToBRFV nucleic acids;
(e) a forward primer and a reverse primer comprising or consisting of nucleotide sequences that are complements of the corresponding nucleotide sequences of the forward primer and reverse primer of a primer set selected from the group consisting of (a)-(d); or
(f) a combination of a primer set selected from the group consisting of (a)-(e).

4. The method of claim 3, wherein the oligonucleotide probe is selected from the group consisting of:
(a) a probe comprising or consisting of the sequence of SEQ ID NO:3,
(b) a probe comprising or consisting of a sequence selected from the group consisting of SEQ ID NO:6 and SEQ ID NO:7,
(c) a probe that differs from the corresponding nucleotide sequence of a probe selected from the group consisting of (a) and (b) in that the probe has up to three nucleotide changes compared to the corresponding sequence, wherein the probe is capable of hybridizing to and detecting the ToBRFV RNA or amplicon thereof, and
(d) a combination of probes selected from the group consisting of (a)-(c).

5. The method of claim 4, wherein each probe is detectably labeled with a different fluorophore, optionally wherein each probe is detectably labeled with a 5′-fluorophore and a 3′-quencher.

6. The method of claim 1, wherein the sample is a stool sample or an environmental sample.

7. The method of claim 6, wherein the environmental sample is a water sample, earth sample, sludge sample, air sample, surface sample, or a fomite sample.

8. The method of claim 7, wherein the water sample is wastewater, stormwater, ocean water, lake water, river water, creek water, drinking water, recreational water, ground water, source water, stored water, seepage water, surface water, or water from a water distribution system or sewage and waste water treatment system.

9. The method of claim 1, wherein said amplifying comprises performing polymerase chain reaction (PCR) or isothermal amplification.

10. The method of claim 9, wherein the PCR is reverse transcriptase polymerase chain reaction (RT-PCR), droplet digital polymerase chain reaction (ddPCR), or quantitative PCR (qPCR).

11. The method of claim 1, further comprising quantifying the amount of the ToBRFV RNA or amplicon thereof, wherein the amount of the ToBRFV RNA or amplicon thereof is indicative of the amount of human feces in the sample.

12. The method of claim 11, further comprising detecting a pathogen in the sample, wherein the amount of ToBRFV RNA or amplicon thereof is used to estimate the amount of the pathogen per the amount of the human feces in the sample.

13. The method of claim 1, further comprising performing microbial source tracking (MST), wherein the ToBRFV is used as an MST marker of human feces, optionally wherein the ToBRFV is used to distinguish fecal contamination from a human and a non-human animal.

14. A composition comprising:
(a) a forward primer comprising or consisting of the sequence of SEQ ID NO:1 and a reverse primer comprising the sequence of SEQ ID NO:2;
(b) a forward primer comprising or consisting of the sequence of SEQ ID NO:4 and a reverse primer comprising the sequence of SEQ ID NO:5;
(c) a forward primer and a reverse primer each comprising at least 10 contiguous nucleotides from the corresponding nucleotide sequences of the forward primer and reverse primer of a primer set selected from the group consisting of (a) and (b);
(d) a forward primer and a reverse primer comprising at least one nucleotide sequence that differs from the corresponding nucleotide sequence of the forward primer or reverse primer of a primer set selected from the group consisting of (a)-(c) in that the primer has up to three nucleotide changes compared to the corresponding sequence, wherein the primer is capable of hybridizing to and amplifying ToBRFV nucleic acids in a nucleic acid amplification assay;
(e) a forward primer and a reverse primer comprising nucleotide sequences that are complements of the corresponding nucleotide sequences of the forward primer and reverse primer of a primer set selected from the group consisting of (a)-(d); or
(f) a combination of a primer set selected from the group comprising (a)-(e).

15. The composition of claim 14, further comprising at least one oligonucleotide probe selected from the group consisting of:
(a) a probe comprising or consisting of the sequence of SEQ ID NO:3,
(b) a probe comprising or consisting of a sequence selected from the group consisting of SEQ ID NO:6 and SEQ ID NO:7,
(c) a probe that differs from the corresponding nucleotide sequence of a probe selected from the group consisting of (a) and (b) in that the probe has up to three nucleotide changes compared to the corresponding sequence, wherein the probe is capable of hybridizing to and detecting the ToBRFV RNA or amplicon thereof, and
(d) a combination of probes selected from the group comprising (a)-(c).

16. The composition of claim 15, wherein the composition comprises:
(a) a forward primer comprising or consisting of the sequence of SEQ ID NO:1, a reverse primer comprising or consisting of the sequence of SEQ ID NO:2, and a probe comprising or consisting of the sequence of SEQ ID NO:3; and
(b) a forward primer comprising or consisting of the sequence of SEQ ID NO:4, a reverse primer comprising or consisting of the sequence of SEQ ID NO:5, and a probe comprising or consisting of the sequence of SEQ ID NO:3.

17. The composition of claim 15, wherein each probe is detectably labeled with a different fluorophore, optionally wherein each probe is detectably labeled with a 5′-fluorophore and a 3′-quencher.

18. A method of performing wastewater surveillance of a pathogen, the method comprising:
isolating nucleic acids from a sample of wastewater comprising human feces, wherein if ToBRFV RNA is present, said nucleic acids comprise a target sequence;
amplifying the nucleic acids using a set of primers capable of selectively amplifying at least a portion of a movement protein (Mo) encoding gene or at least a portion of an RNA dependent RNA polymerase (RdRP) encoding gene;
detecting the presence of the amplified nucleic acids using a detectably labeled oligonucleotide probe sufficiently complementary to and capable of hybridizing with the ToBRFV RNA or amplicon thereof, if present, as an indication of the presence or absence of the ToBRFV in the sample of wastewater, wherein said primers and said probe are capable of selectively hybridizing to the target sequence from the ToBRFV, wherein said detecting the presence of the ToBRFV in the sample indicates that human feces is present in the sample of wastewater;
quantifying the amount of the ToBRFV RNA or amplicon thereof, wherein the amount of the ToBRFV RNA or amplicon thereof is indicative of the amount of the human feces in the sample of wastewater;
quantifying the amount of the pathogen in the sample of wastewater; and
dividing the amount of the pathogen by the amount of the human feces, as determined from said quantifying the amount of the ToBRFV RNA or amplicon thereof, to provide a normalized value for the amount of the pathogen per the amount of human feces in the wastewater.

19. The method of claim 18, further comprising correlating the normalized value for the amount of the pathogen per the amount of human feces in the wastewater with incidence of an infectious disease caused by the pathogen.

20. The method of claim 19, wherein the pathogen is a virus, a bacterium, a fungus, or a parasite.

21. The method of claim 20, wherein the virus is an enterovirus, a rotavirus, a parvovirus-like virus, an astrovirus, a calicivirus, an adenovirus, a norovirus, or a coronavirus.

22. The method of claim 21, wherein the virus is severe acute respiratory syndrome coronavirus 2 (SARS-CoV-2), poliovirus, coxsackie A virus, coxsackie B virus, echovirus, hepatitis A virus, hepatitis B virus, hepatitis E virus, herpesvirus, influenza A, influenza B, respiratory syncytial virus (RSV), monkeypox virus (MPXV), dengue virus, yellow fever virus, zika virus, or varicella-zoster virus.

23. The method of claim 20, wherein the bacterium is an antibiotic-resistant bacterium.

24. The method of claim 18, wherein the set of primers comprises:
(a) a forward primer comprising the sequence of SEQ ID NO:1 and a reverse primer comprising the sequence of SEQ ID NO:2;
(b) a forward primer comprising the sequence of SEQ ID NO:4 and a reverse primer comprising the sequence of SEQ ID NO:5;
(c) a forward primer and a reverse primer each comprising at least 10 contiguous nucleotides from the corresponding nucleotide sequences of the forward primer and reverse primer of a primer set selected from the group consisting of (a) and (b);
(d) a forward primer and a reverse primer comprising at least one nucleotide sequence that differs from the corresponding nucleotide sequence of the forward primer or reverse primer of a primer set selected from the group consisting of (a)-(c) in that the primer has up to three nucleotide changes compared to the corresponding sequence, wherein the primer is capable of hybridizing to and amplifying the ToBRFV nucleic acids;
(e) a forward primer and a reverse primer comprising nucleotide sequences that are complements of the corresponding nucleotide sequences of the forward primer and reverse primer of a primer set selected from the group consisting of (a)-(d); or
(f) a combination of a primer set selected from the group comprising (a)-(e).

25. The method of claim 24, wherein the oligonucleotide probe is selected from the group consisting of:
(a) a probe comprising the sequence of SEQ ID NO:3,
(b) a probe comprising the sequence of SEQ ID NO:6,
(c) a probe that differs from the corresponding nucleotide sequence of a probe selected from the group consisting of (a) and (b) in that the probe has up to three nucleotide changes compared to the corresponding sequence, wherein the probe is capable of hybridizing to and detecting the ToBRFV RNA or amplicon thereof, and
(d) a combination of probes selected from the group comprising (a)-(c).

26. The method of claim 18, wherein said amplifying comprises performing polymerase chain reaction (PCR) or isothermal amplification.

27. The method of claim 18, further comprising amplifying the nucleic acids using a set of primers capable of selectively amplifying at least a portion of a SARS-CoV-2 envelope (E) encoding gene and/or a nucleocapsid protein N2 (N2) encoding gene;
detecting the presence of the amplified nucleic acids using a detectably labeled oligonucleotide probe sufficiently complementary to and capable of hybridizing with the SARS-CoV-2 RNA or amplicon thereof, if present, as an indication of the presence or absence of the SARS-CoV-2 in the sample of wastewater;
quantifying the amount of the SARS-CoV-2 RNA or amplicon thereof, wherein the amount of the SARS-CoV-2 RNA or amplicon thereof is indicative of the amount of the SARS-CoV-2 in the sample of wastewater; and
dividing the amount of the SARS-CoV-2 RNA or amplicon thereof by the amount of the ToBRFV RNA or amplicon thereof, to provide a normalized value for the amount of the SARS-CoV-2 per the amount of human feces in the wastewater.


SEQUENCE LISTING (20 sequences)

SEQ ID NO: 1 - DNA, 22 bp, synthetic construct
  source 1..22: mol_type = other DNA
tcagtgtctgtttggtcgataa

SEQ ID NO: 2 - DNA, 22 bp, synthetic construct
  source 1..22: mol_type = other DNA
ggaacgactttgaactgaaacc

SEQ ID NO: 3 - DNA, 22 bp, synthetic construct
  source 1..22: mol_type = other DNA
agagcggacgaggcaactcttg

SEQ ID NO: 4 - DNA, 23 bp, synthetic construct
  source 1..23: mol_type = other DNA
agccacaagagataatgttcgta

SEQ ID NO: 5 - DNA, 23 bp, synthetic construct
  source 1..23: mol_type = other DNA
acatcagaccttcgtcgataaat

SEQ ID NO: 6 - DNA, 23 bp, synthetic construct
  source 1..23: mol_type = other DNA
cggtaaaggaacacgctgtcagt

SEQ ID NO: 7 - DNA, 24 bp, synthetic construct
  source 1..24: mol_type = other DNA
acggtaaaggaacacgctgtcagt

SEQ ID NO: 8 - DNA, 6393 bp, Tomato brown rugose fruit virus
  source 1..6393: mol_type = genomic DNA
gtatttttgttttacaacatataccaacaacaacaaacaacaaacaacaacattacaattactatttacaactacaatggcatacacacagacagctaccacatccgctttgctcgacactgtccgaggtaacaataccttggtcaacgatcttgcgaagcggcgtctttatgacacagcggtcgacgagttcaacgctcgtgatcgcaggcccaaagtaaatttttccaaagtaataagtgaggaacagacgcttattgctactagggcatatccagaattccagataaccttctataatacgcagaacgccgtgcattcgcttgccggtggactacgatccttagaactggaatatctaatgatgcagatcccgtacggatcactcacatatgatataggtgggaattttgcatctcatctgttcaaaggacgggcatatgttcactgctgtatgcccaatcttgatgtccgcgacataatgcggcacgaaggccagaaagacagtatagaattatacctttccaggcttgagcggggcaacaaagttgtcccaaatttccaaaaggaagcttttgacagatacgctgaaacgccagacgaagttgtctgtcacagtaccttccaaacgtgtacgcaccagcaggtggaaaacacaggcagggtgtatgctattgcattgcacagtatatacgatatacctgctgatgaattcggagcggcacttttaaggaaaaatgtccatgtttgttacgccgccttccacttttccgagaatttacttctcgaagattcacacgtcaaccttgacgaaatcaacgcgtgtttttcgcgtgatggagacaagctgactttttctttcgcatctgagagcactttaaattattgtcatagttattctaatattttaaaatacgtgtgcaaaacttacttcccggcatctaatagagaggtctacatgaaggagtttttggtcaccagggttaacacctggttttgtaagttttctaggatagatacttttttattatacaagggggtagcccacaaaggtgtaaatagtgagcaattttacagcgcaatggaagatgcatggcactacaaaaagactcttgcaatgtgtaacagcgagaggattcttcttgaagattcctcatcggtcaattactggttcccaaaaatgagagatatggtcatagttcctctattcgacatatctctcgacaccagtaaaaggacccgcaaagaagtcttagtgtcaaaggattttgtattcacagttttaaatcacattcgcacttatcaagccaaggcacttacatactccaatgttttatcctttgtcgaatcaattcgttcaagggtaattatcaacggagtgactgccaggtctgagtgggatgttgacaaatctcttttgcaatccttgtccatgacatttttcttgcatactaagcttgccgttttaaaagacgaattgttaatcagcaagtttagtttggggccaaaatcagtaagccagcatgtatgggatgagatttccctggcttttggaaacgcatttccatcgatcaaggagagactgctaaatcggaaactaattaaagtgtcgggagacgcattagaaatcagggtgcctgatttatatgtgacttttcacgatagattagtgactgagtacaaaacatcggtggatatgccagtgcttgatatcagaaagagaatggaggagactgaggttatgtacaatgcattgtctgagctatctgtgctcaaggagtcggacaagttcgacgttgatgttttttcccggatgtgccagactttggaggtagacccaatgactgcagcaaaggttattgtggcagtgatgagcaacgagagcggactgactcttacattcgaacagccaactgaagcaaatgtcgcattggcacttaaagattcagaaaaagcctctgagggtgcactagtggttacttctagagatgttgaagaaccatccatgaagggttcaatggcaagaggagagttacaattggccggtctgtctggagaccaaccagagtcttcctatactcggaacgaggaaatagagtcattagagcaattccacatggcaacggctagttcgttaattcggaaacagatgagttcgattgtgtacacgggccccattaaagttcagcaaatgaaaaactttattgatagcctggtagcatcactctctgctgcggtgtcgaacctagtcaagatcctaaaggatacagctgctatagatctcgaaacccgtcagaagtttggagtcttagatgttgcgaccaaaagatggttaattaaacctttagccaagaatcacgcatggggcgttattgaaacacatgctaggaagtaccacgttgcacttttggagtatgatgagcatggagtggtaacttgcgacagttggagaagggtggccgtgagttctgagtcaatggtttattctgatatggcaaagctcagaacactgaggagattattaagagatggtgagcctcatgtcagcagtgctaaagtcgtcctagttgacggtgtcccgggttgtggaaagacaaaagagattctctcgaaagtaaattttgaggaagatctaatcttagtaccgggtaagcaggctgctgaaatgataaagaggcgtgctaatgcgtcaggaataattcaagccacaagagataatgttcgtactgttgattcatttataatgaattacggtaaaggaacacgctgtcagttcaaaaggttatttatcgacgaaggtctgatgttgcacactggttgtgtgaattttcttgtttctatgtctctgtgcgaaattgcatatgtttatggagacacacaacaaattccatacatcaacagagtatccggttttccgtaccctgcacattttgcaaaaatagaggttgatgaggtggaaactcgcagaactacgctgcgttgtccagccgacattacccactatcttaacagaaggtacgaaggatatgtcatgtgtacatcgtcggttaaaaagtcagtttctcaggaaatggtgagcggggccgcaatgatcaatcctgtatctaagccattgaatgggaaagttttgactttcactcagtctgataaagaggcgctgctttctcgaggatatacggacgtccatacagtacatgaggtacaaggtgagacatatgcagatgtgtcgttggtcagattgactccgacacctgtatctatcatcgcaggagatagtccgcacgttctcgtagctttgtcaaggcatacccaaacattgaagtattacaccgtagtgatggatcctcttgtaagtataattagggatttagaaaaacttagttcttacttgttagatatgtataaagtagatgcagggacccaatagcaattacaggtagactccgtgtttaaaggttctaatctttttgttgcagcaccaaagactggagatatctcagatatgcaattttactatgataagtgtctcccaggtaatagcaccatgttaaataactatgatgctgttaccatgaggttgactgacatttctcttaatgtcaaagattgcatattggatttctctaagtctgtggctgcaccgaaggatccgatcaaaccactgattccgatggtacgaacggcggcagaaatgccacgccagactggactattggaaaatttggtggcgatgatcaaaagaaactttaattcaccggagttatcaggaataatcgacattgagaatactgcatctttagtagtagataaattttttgatagttacttgcttaaagaaaaaagaaaaccaaataaaaatgtttctttattttgtagagagtctctcaatagatggttagagaagcaggagcaagtgaccattggtcagcttgcagattttgattttgtggatcttcctgccgttgatcagtacaggcatatgattaaagcgcaacctaagcagaagctggatacatcaattcaaagcgaatatccggccttgcagacgattgtgtatcattcgaaaaagatcaacgcaatcttcggtcctttgttcagtgagctcacaaggcaaatgctcgaaagcatagactcaagtaagtttttgttctttacaaggaagacgccagctcaaattgaggatttcttcggagatctcgatagccatgtccctatggatatcttggagttggatatttcgaagtatgacaaatctcagaacgagttccactgtgcagtagagtatgaaatatggagaagacttggattagaagattttctgggagaagtttggaaacaaggccacaggaaaactactcttaaagattacacagctggtattaaaacgtgtttatggtaccagagaaagagtggggacgttacaacattcatcggtaatacggtgattattgctgcttgtttagcttccatgttgcccatggagaaaataatcaaaggtgcattttgcggagatgacagtttactatacttcccaaaaggttgtgagtttcctgacatacagcatacagccaaccttatgtggaatttcgaggctaagctattcagaaagcagtatggttatttctgtggaaggtacgtgatacatcatgacagagggtgtattgtttattatgaccctttgaagttgatttctaaacttggtgctaaacacatcaaggattgggatcacttagaagagttcagaagatccctttgtgatgttgcaaattcgttgaacaactgtgcgtattacacgcagttggacgacgctgtgagtgaggtccataaaaccgcacccccgggttcgtttgtatataaaagtttagttaaatatctgtccgataaggttctttttagaagtttgtttatagatggctcttgttaagggtaaagtcaatattaatgagttcatagacttgtcaaaatcagaaaaatttcttccgtctatgttcacacctgttaagagtgtcatgatctccaaggttgataagatattggttcatgaagatgaatctttgtccgaagtcaatttactcaaaggtgtaaaactcattgatggtggctatgtacatcttgctggtcttgtggtgacaggtgaatggaatttgccagataattgtcgtggtggtgtcagtgtctgtttggtcgataagagaatggagagagcggacgaggcaactcttgcttcatactataccgcagcggctaagaaaaggtttcagttcaaagtcgttccaaattacaacatcactaccaaggacgcagaaaaggcagtttggcaagtactagttaatattagaaatgttaaaattgctgcgggttactgtccgctgtcattagaatttgtgtcagtgtgtattgtttataaaaatattataaaactcggtttgagagagaaaattacgagcgtcacggatggagggcccatggaactatcagaagaagttgttgatgagttcatggaagaagtcccgatgtctgtaaggcttgcaaaatttcgttcgaagaccggaaaaaagtttagtagtaaaagtgagaataatagtggtaataataggccgaaaccagacaaaaaccaaaggaaggaaaagggtttaaaagttagggttgagaaggataatttaattgataatgaattggagacttacgtcgccgattcagattcgtattaaatatgtcttacacaatcgcaactccatcgcaatttgtgtttttgtcatcagcatgggccgaccctatagaattaataaatttatgtactaattcactaggtaatcagttccaaacacaacaagctagaacaaccgttcaacggcaatttagcgaagtgtggaaacctgtccctcaagtcactgttaggtttcctgacagtggttttaaggtgtataggtacaatgcggtactagatcctctagttactgctttgttaggagctttcgatactagaaataggattatagaagtcgaaaatcaggcgaacccgacaaccgccgaaacgttagacgctactcgtagagtagatgacgcaacggtggctataaggagcgctataaataatttagtagtagaattggtcaaaggaacaggtttgtacaatcagagcacatttgaaagtgcatccggtttacaatggtcctctgcacctgcatcttgagataatcgagatgcttaaataacagattgtgtctgcaaacacacgtggtacgtacgataacgtatagtgtttttccctccacttaaatcgaagggtagtgtcttggagcgcgcgggacaaatgtgtatggttcatacacatccgtaggcacgtaataaagcgagggattcgaattcccccggaacccccggaggggccca

SEQ ID NO: 9 - DNA, 24 bp, synthetic construct
  source 1..24: mol_type = other DNA
gagtggtttgaccttaacgtttga

SEQ ID NO: 10 - DNA, 20 bp, synthetic construct
  source 1..20: mol_type = other DNA
ttgtcggttgcaatgcaagt

SEQ ID NO: 11 - DNA, 18 bp, synthetic construct
  source 1..18: mol_type = other DNA
ctggaagttggtggagtt

SEQ ID NO: 12 - DNA, 21 bp, synthetic construct
  source 1..21: mol_type = other DNA
attatcggcctaacatacatc

SEQ ID NO: 13 - DNA, 26 bp, synthetic construct
  source 1..26: mol_type = other DNA
acaggtacgttaatagttaatagcgt

SEQ ID NO: 14 - DNA, 22 bp, synthetic construct
  source 1..22: mol_type = other DNA
atattgcagcagtacgcacaca

SEQ ID NO: 15 - DNA, 20 bp, synthetic construct
  source 1..20: mol_type = other DNA
ttacaaacattggccgcaaa

SEQ ID NO: 16 - DNA, 18 bp, synthetic construct
  source 1..18: mol_type = other DNA
gcgcgacattccgaagaa

SEQ ID NO: 17 - DNA, 16 bp, synthetic construct
  source 1..16: mol_type = other DNA
cctaccgaagcaaatg

SEQ ID NO: 18 - DNA, 28 bp, synthetic construct
  source 1..28: mol_type = other DNA
ccttcatatctatacacatcaagttgtt

SEQ ID NO: 19 - DNA, 26 bp, synthetic construct
  source 1..26: mol_type = other DNA
acactagccatccttactgcgcttcg

SEQ ID NO: 20 - DNA, 23 bp, synthetic construct
  source 1..23: mol_type = other DNA
acaatttgcccccagcgcttcag